(19) United States
(12) Patent Application Publication
(10) Pub. No.: US 2026/0137807 A1
(43) Pub. Date: May 21, 2026

(54) COMPOSITIONS AND METHODS FOR DELIVERING A MACROMOLECULE TO A CELL

(71) Applicants: AstraZeneca AB, Södertälje (SE); Karolinksa Institute, Solna (SE)

(72) Inventors: Shalini ANDERSSON, Södertälje (SE); Anna COLLÉN, Södertälje (SE); Per-Erik STRÖMSTEDT, Södertälje (SE); Anders DAHLÉN, Södertälje (SE); Ola ENGKVIST, Södertälje (SE); Jeremy BOST, Solna (SE); Samir EL ANDALOUSSI, Solna (SE); Yesid ESTUPINAN, Solna (SE); Oskar GUSTAFSSON, Solna (SE)

(21) Appl. No.: 19/121,447

(22) PCT Filed: Oct. 17, 2022
(86) PCT No.: PCT/IB2023/060479

§ 371 Date: Apr. 16, 2025

Related U.S. Application Data
(60) Provisional application No. 63/379,912, filed on Oct. 18, 2022.

(51) Int. Cl. A61K 48/00; A61K 31/7125; C07D 401/14; C12N 9/12; C12N 9/78; C12N 15/113

(52) U.S. Cl. CPC A61K 48/0033; A61K 31/7125; A61K 48/0058; A61K 48/0083; C07D 401/14; C12N 9/1241; C12N 9/78; C12N 15/1137; C12Y 305/04004; C12N 2310/14; C12N 2310/315; C12N 2310/321; C12N 2310/333; C12N 2310/3341; C12N 2310/335; C12N 2310/351; C12N 2310/3515; C12N 2320/33

(57) ABSTRACT

Compounds and methods are provided for facilitating delivery of an oligonucleotide, for example, into a nucleus and/or cytosol of a cell.

### FIELD OF THE DISCLOSURE

The present disclosure relates to compositions and methods for delivering a macromolecule, e.g., an oligonucleotide, a polypeptide, or a combination thereof, to a cell, for instance, to compositions and methods for delivering a macromolecule, e.g., an oligonucleotide, a polypeptide, or a combination thereof, to the cytosol and/or nucleus of a cell.

### BACKGROUND OF THE DISCLOSURE

Antisense oligonucleotides (ASOs) are short, exogenous, single stranded DNA or RNA with a sequence that is complementary to a nucleotide sequence of a target nucleic acid. Classic single stranded antisense oligonucleotides (ASOs) can act in the nucleus to cleave mRNAs via an RNase H dependent mechanism. (Juliano, R L (2016) Nuc. Acids. Res. 44(14):6518-6548). Many therapeutic ASOs are ‘gapmers’, which have a central DNA region that supports RNase H activity that is flanked by chemically modified ends to increase affinity and reduce susceptibility to nucleases. (Bennett et al. (2010) Annu. Rev. Pharmacol. Toxicol. 50:259-293).

Splice switching oligonucleotides (SSOs) are another form of antisense oligonucleotides that hybridize with pre-mRNA to disrupt transcript splicing by blocking RNA-RNA base pairing or protein-RNA binding interactions that occur between components of the splicing machinery (Havens and Hastings (2016) Nucleic Acids Res. 44(14); 6549-63). SSOs can be designed to induce intron and exon inclusion or exclusion, ultimately restoring or inhibiting protein function or re-directing splicing to produce alternative protein isoforms. Additionally, SSOs can be used to mask aberrant splice sites, thereby restoring normal alternative splicing to produce functional proteins. Theoretically, any pre-mRNA sequence could be targeted with SSOs, but to date, only four SSOs have been FDA approved. (See e.g., Neil and Bisaccia (2019) J. Pediatr. Pharmacol. Ther. 24(3):194-203; Kim et al. (2019) N. Engl. J Med. 381:1644-1652). It is estimated that up to 70% of human genes undergo alternative splicing, and 50% of human genetic diseases arise from mutations that affect splicing. (Bauman et al., (2009) Oligonucleotides. 19(1):1-13) Several diseases, including Spinal Muscular Atrophy (SMA) and Duchenne Muscular Dystrophy (DMD), currently lack any efficacious treatments which target the underlying genetic defect. (Bestas et al. (2014) Nucleic Acid Ther. 24(1):13-24). SSOs are a promising therapeutic approach to target the underlying causes of these diseases.

RNA interference (RNAi) is an endogenous regulatory pathway for control of gene expression in which short (approx. 15-22 bp) double-stranded RNA fragments, known as small interfering RNAs (siRNAs), are loaded into an RNA-induced silencing complex (RISC) to cleave target mRNA in a sequence-dependent manner. (Gavriolv and Saltzman (2012) Yale J. Biol. Med. 85(2):187-200).

Although nucleic acid-based therapeutics are gaining attention as a promising approach for treatment of a variety of diseases and disorders, many have failed to meet therapeutic end points, often due to challenges with effective methods for in vivo delivery. (Juliano, R L (2016) Nuc. Acids. Res. 44(14):6518-6548). One hindrance to the widespread use of oligonucleotide therapeutics is the inability of the oligonucleotide to escape endosomal compartments and reach the cytosol or nucleus in sufficient concentrations. (Juliano et al. (2008) Nucleic Acids Res. 36(12):4158-4171).

Many recent developments focus on increasing cellular uptake and endosomal release of therapeutic oligonucleotides, for example, via chemical conjugation to ligands or encapsulation in synthetic nanoparticles. (Barton and Medzhitov (2002) Proc. Natl. Acad. Sci. U.S.A. 99(23):14943-5; Johannes and Lucchino (2018) Nucleic Acid Ther. 28(3):178-193). Historically, use of nano-carriers was believed to be required to facilitate cellular uptake of polyanionic macromolecules such as ASOs. However, it has been discovered that single-stranded oligonucleotides are spontaneously endocytosed by cells, in the absence of carriers, by a process referred to as gymnosis. (Stein et al. (2009) Nucleic Acids Res. 38(1): 10.1093/nar/gkp841).

Endosomolytic small molecule compounds (SMCs) are compounds that facilitate the release of gymnotically delivered oligonucleotides that might otherwise accumulate in endosomes or lysosomes. Some endosomolytic SMCs induce endosomal membrane destabilization by buffering the lumen of endosomes as the luminal pH decreases with endosomal maturation. The increase in luminal pH occurs quickly and can be reversible with proper dosing. (Maxfield, F. R. (1982) J. Cell Biol. 95(2):676-681). This buffering leads to an increase of luminal osmotic pressure, engorging the endosome and triggering membrane rupture, ultimately allowing the endosomal cargo to leak into the cytosol.

Chloroquine and derivatives thereof have been widely used to enhance activity of oligonucleotide-containing nanoparticles by promoting endosomal release. Although these compounds display great potency in vitro, high micromolar concentration ranges are typically required and there is a narrow window between effective and toxic concentrations. (Yang et al. (2015) Nucleic Acids Res. 43(4):1987-96; Wang et al. (2017) ACS Chem. Biol. 12(8):1999-2007). For example, chloroquine induces leakage between 40-100 μM (Lönn et al. (2016) Sci. Rep. 6:32301; Heath et al. (2019) Nanomedicine. 14(21):2799-2814).

### SUMMARY OF THE DISCLOSURE

Compositions and methods for delivering a macromolecule, e.g., an oligonucleotide, a polypeptide, or a combination thereof, to a cell are provided. In one aspect, a compound of Formula I is provided.

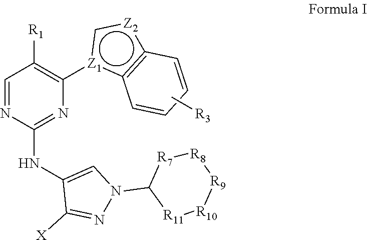

wherein
one of Z₁ and Z₂ is N, and the other is C;
R₁ is hydro, halo, C₁-C₄ alkyl, —OR₄, —C(═O)NR₅R₅, —CO₂R₆, or cyano;
X is hydro, C₁-C₄ alkyl, or —OR₂;
R₂ is hydro or C₁-C₄ alkyl;
R₃ is hydro, halo, C₁-C₄ alkyl, —(CH₂)_{y}OH, —OR₄, —C(═O)NR₅R₅, —CO₂R₆, or cyano;
R₄ is C₁-C₄ alkyl;
each R₅ is independently hydro or C₁-C₄ alkyl;
R₆ is hydro or C₁-C₄ alkyl;
R₇, R₈, R₉, R₁₀, R₁₁ are each independently CHR₁₂, CR₁₂R₁₇ or NR₁₃;
R₁₂ is hydro, C₁-C₄ alkyl, —OR₁₄, or —CO₂R₁₅;
R₁₃ is hydro, C₁-C₄ alkyl, —(CH₂)_{y}OH, —OR₁₄, —CO₂R₁₅, or —C(═O)R₁₆;
R₁₄ is hydro or C₁-C₄ alkyl;
R₁₅ is hydro or C₁-C₄ alkyl;
R₁₆ is hydro or C₁-C₄ alkyl;
R₁₇ is hydro or C₁-C₄ alkyl;
y is 0, 1, 2, or 3; and
wherein one or more of the alkyl are optionally substituted with one or more halo, or a pharmaceutically acceptable salt thereof.

In another aspect, a compound of Formula Ia is provided.

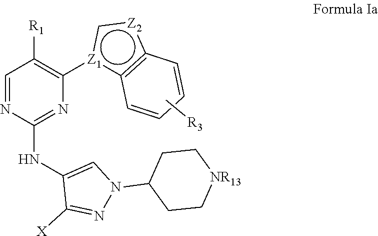

wherein
one of Z₁ and Z₂ is N, and the other is C;
R₁ is hydro, halo, C₁-C₄ alkyl, —OR₄, —C(═O)NR₅R₅, —CO₂R₆, or cyano;
X is hydro, C₁-C₄ alkyl, or —OR₂;
R₂ is hydro or C₁-C₄ alkyl;
R₃ is hydro, halo, C₁-C₄ alkyl, —(CH₂)_{y}OH, —OR₄, —C(═O)NR₅R₅, —CO₂R₆, or cyano;
R₄ is C₁-C₄ alkyl;
each R₅ is independently hydro or C₁-C₄ alkyl, e.g., methyl or ethyl;
R₆ is hydro or C₁-C₄ alkyl, e.g., methyl or ethyl;
R₁₃ is hydro, C₁-C₄ alkyl, —(CH₂)_{y}OH, —OR₁₄, —CO₂R₁₅, or —C(═O)R₁₆;
R₁₄ is hydro or C₁-C₄ alkyl;
R₁₅ is hydro or C₁-C₄ alkyl;
R₁₆ is hydro or C₁-C₄ alkyl; and
wherein one or more of the alkyl are optionally substituted with one or more halo, or a pharmaceutically acceptable salt thereof.

In another aspect, a compound of Formula Ib or Formula Ic is provided.

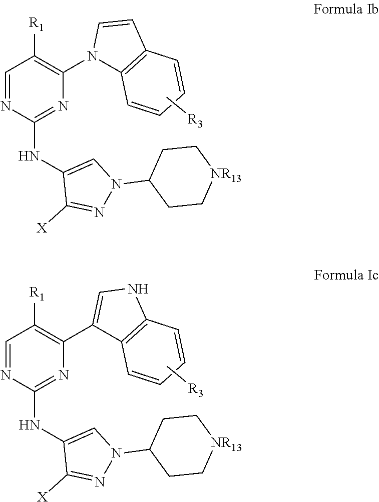

wherein R₁, X, R₃, and R₁₃ are as defined above.

In another aspect, a compound of Formula I, Ia, Ib, or Ic is provided, wherein
R₁ is hydro, halo, C₁-C₄ alkyl, —OR₄, —C(═O)NR₅R₅, —CO₂R₆, or cyano;
X is hydro, C₁-C₄ alkyl, or —OR₂,
R₂ is hydro or C₁-C₄ alkyl;
R₃ is hydro, halo, C₁-C₄ alkyl, —(CH₂)_{y}OH, —OR₄, —C(═O)NR₅R₅, —CO₂R₆, or cyano;
R₄ is C₁-C₄ alkyl;
R₅ is hydro, methyl or ethyl,
R₆ is hydro, methyl or ethyl,
R₁₃ is hydro, C₁-C₄ alkyl, —(CH₂)_{y}OH, —OR₁₄, —CO₂R₁₅, or —C(═O)R₁₆;
R₁₄ is hydro or C₁-C₄ alkyl;
R₁₅ is hydro or C₁-C₄ alkyl;
R₁₆ is hydro or C₁-C₄ alkyl;
wherein one or more of the alkyl are optionally substituted with one or more halo, or a pharmaceutically acceptable salt thereof.

In some aspects, R₁ is cyano, halo, or C₁-C₄ alkyl optionally substituted with one or more chloro or fluoro. In one aspect, R₁ is cyano, methyl, ethyl, trifluoromethyl, difluoromethyl, fluoromethyl, trichloromethyl, dichloromethyl, chloromethyl, fluoro, chloro, or bromo. In one aspect, R₁ is cyano, bromo, chloro, fluoro, or trifluoromethyl. In one aspect, R₁ is fluoro.

In one aspect, X is —OR₂, and R₂ is methyl, ethyl, or isopropyl. In one aspect, X is hydro.

In one aspect, R₃ is hydro; cyano; C₁-C₄ alkyl optionally substituted with one or more chloro, fluoro, or hydroxy; or —OR₄, wherein R₄ is C₁-C₄ alkyl.

In one aspect, R₇, R₈, R₁₀, and R₁₁ are each independently CHR₁₂ or CR₁₂R₁₇, and R₉ is NR₁₃. In one aspect, each R₁₂ is hydro. In one aspect, R₁₃ is hydro, C₁-C₄ alkyl, —C(═O)R₁₆, or —(CH₂)_{y}OH, wherein y is 1, 2 or 3. In one aspect, R₁₃ is —(CH₂)₃OH. In one aspect, R₁₃ is methyl.

In one aspect, R₁ is halo, X is —OR₂, R₂ is ethyl or methyl, R₃ is cyano, and R₁₃ is hydro, C₁-C₄ alkyl, —C(═O)CH₃, or —(CH₂)₃OH. In one aspect, R₁ is haloalkyl, X is —OR₂, R₂ is ethyl or methyl, R₃ is cyano, and R₁₃ is hydro or methyl. In one aspect, R₁ is halo, X is —OR₂, R₂ is ethyl or methyl, R₃ is methyl or methoxy, and R₁₃ is hydro. In one aspect, R₁ is halo, X is —OR₂, R₂ is C₁-C₄ alkyl, R₃ is hydro, and R₁₃ is hydro. In one aspect, R₁ is haloalkyl, X is —OR₂, R₂ is ethyl or methyl, R₃ is hydro, and R₁₃ is hydro or methyl.

In one aspect, R₁ is halo, X is hydro, R₃ is cyano, and R₁₃ is hydro. In one aspect, R₁ is cyano, X is —OR₂, R₂ is ethyl or methyl, R₃ is haloalkyl, and R₁₃ is hydro. In one aspect, R₁ is ethyl or methyl, X is —OR₂, R₂ is ethyl or methyl, R₃ is hydro, and R₁₃ is hydro. In one aspect, R₁ is halo, X is —OR₂, R₂ is ethyl or methyl, R₃ is (CH₂)_{y}OH, and R₁₃ is hydro, wherein y is 0, 1, 2, or 3.

In some embodiments, disclosed are the compounds of Table 1, or a pharmaceutically acceptable salt thereof.

TABLE 1
Exemplified Compounds

| | | MS | |
|---|---|---|---|
| Cmpd | Chemical Structure | (ESI+) | NMR |
| 2 | 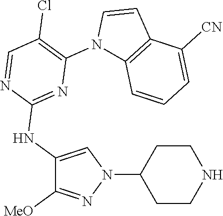 [SMILES: COc1nn(C2CCNCC2)cc1Nc1ncc(Cl)c(-n2ccc3c(C#N)cccc32)n1] | 449.1 | 1H NMR: (400 MHz DMSO-d6): δ 8.44(s, 1H), 8.08 (s, 1H), 7.85 (d, J = 8.0 Hz, 1H), 7.73 (s, 1H), 7.61 (d, J = 8.0 Hz, 1H), 7.34 (t, J = 8.0 Hz , 1H), 3.90 (s, 1H), 3.77 (m, 3H), 2.96 (s, 2H), 1.84 (s, 2H), 1.67-1.60 (m, 2H). |
| 3 | 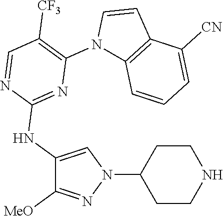 [SMILES: COc1nn(C2CCNCC2)cc1Nc1ncc(C(F)(F)F)c(-n2ccc3c(C#N)cccc32)n1] | 483.1 | 1H NMR: (400 MHz DMSO-d6): δ 8.84 (brs, 1H), 7.84 (d, J = 8.0 Hz, 1H), 7.72 (s, 1H), 7.56 (d, J = 7.2 Hz), 7.33 (t, J = 8 Hz, 1H), 3.91 (brs, 1H), 3.90 (s, 3H), 2.99 (s, 2H), 2.53-2.50 (m, 1H), 1.85 (s, 1H), 1.62 (brs, 1H). |
| 4 | 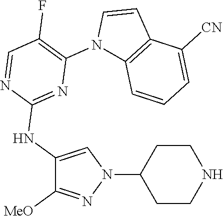 [SMILES: COc1nn(C2CCNCC2)cc1Nc1ncc(F)c(-n2ccc3c(C#N)cccc32)n1] | 433.1 | 1H NMR: (400 MHz DMSO-d6): δ 8.81 (brs, 1H), 8.60 (d, J = 4.0 Hz, 1H), 8.08 (s, 1H), 7.73 (s, 1H), 7.32 (brs, 1H), 6.95 (d, J = 4.0 Hz, 1H), 3.99-3.94 (m, 1H), 3.79 (s, 1H), 3.01 (d, J = 8.0 Hz, 1H), 2.57-2.54 (m, 2H), 1.90-1.87 (m, 2H), 1.71-1.68 (m, 2H). |
| 5 | 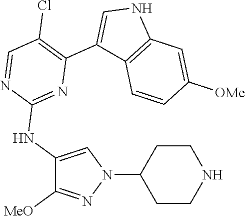 [SMILES: COc1ccc2c(-c3nc(Nc4cn(C5CCNCC5)nc4OC)ncc3Cl)c[nH]c2c1] | 454.0 | 1H NMR (300 MHz, MeOD-d4): δ ppm 2.02- 2.29 (m, 4 H) 3.01-3.16 (m, 2 H) 3.46 (dt, J = 12.81, 3.58 Hz, 2 H) 3.75 (s, 3 H) 3.81 (s, 3 H) 4.10-4.31 (m, 1 H) 6.67 (d, J = 8.48 Hz, 1 H) 6.89 (d, J = 2.26 Hz, 1 H) 7.67 (s, 1 H) 8.12 (s, 2 H) 8.41 (s, 1 H) |
| 6 | 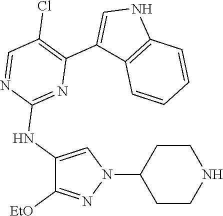 [SMILES: CCOc1nn(C2CCNCC2)cc1Nc1ncc(Cl)c(-c2c[nH]c3ccccc23)n1] | 438.0 | 1H NMR (400 MHz DMSO-d6): δ ppm 11.82 (br s, 1 H), 8.48 (s, 1 H), 8.30 (s, 2 H), 7.70 (s, 1 H), 7.46 (d, J = 7.54 Hz, 1 H), 7.18 (t, J = 8.01 Hz, 1 H), 6.91-7.10 (m, 1 H), 4.15 (q, J = 7.10 Hz, 2 H), 3.97 (dd, J = 15.64,7.54 Hz, 1 H), 3.02 (d, J = 18.84 Hz, 2 H), 2.55-2.67 (m, 2 H), 1.84-2.00 (m, 2 H), 1.56-1.84 (m, 2 H), 1.22 (t, J = 6.97 Hz, 3 H) |
| 7 | 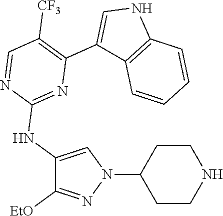 [SMILES: CCOc1nn(C2CCNCC2)cc1Nc1ncc(C(F)(F)F)c(-c2c[nH]c3ccccc23)n1] | 472.1 | 1H NMR: (400 MHz DMSO-d6): δ 9.03- 8.88 (s, 1H), 8.60 (s, 1H), 8.52-7.96 (s, 1H), 7.85 (s, 1H), 7.74 (s, 1H), 7.45-7.43 (s, 1H), 7.10-6.96 (m, 2H), 4.30 (s, 2H), 3.85 (s, 1H), 3.02-2.99 (m, 2H), 2.66-2.55 (m, 2H), 1.88 (m, 2H), 1.70-1.67 (m, 2H), 1.26-1.17 (m, 3H). |
| 8 | 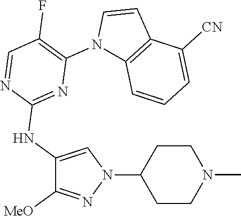 [SMILES: COc1nn(C2CCN(C)CC2)cc1Nc1ncc(F)c(-n2ccc3c(C#N)cccc32)n1] | 447.2 | 1H NMR (500 MHz, DMF-d7): δ 1.94-2.1 (m, 6H), 2.24 (s, 3H), 2.89 (d, J = 10.7 Hz, 2H), 3.89 (s, 3H), 3.96 (dd, J = 11.1, 5.4 Hz, 1H), 7.02 (d, J = 3.6 Hz, 1H), 7.44 (s, 1H), 7.79 (d, J = 7.4 Hz, 1H), 7.89 (s, 1H), 8.18 (dd, J = 3.6, 2.2 Hz, 1H), 8.63 (d, J = 4.3 Hz, 1H). |
| 9 | 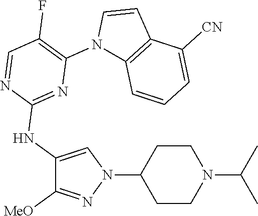 [SMILES: COc1nn(C2CCN(C(C)C)CC2)cc1Nc1ncc(F)c(-n2ccc3c(C#N)cccc32)n1] | 475.4 | 1H NMR (500 MHz, DMF-d7): δ 1.10 (s, 6H), 1.95-2.5 (m, 6H), 3.06 (bs, 2H), 3.89 (s, 3H), 4.03 (m, 1H), 7.03 (d, J = 3.6 Hz, 1H), 7.45 (t, J = 7.7 Hz, 1H), 7.78 (d, J = 7.4 Hz, 1H), 7.90 (s, 1H), 8.16-8.21 (m, 1H), 8.64 (d, J = 4.3 Hz, 1H). |
| 10 | 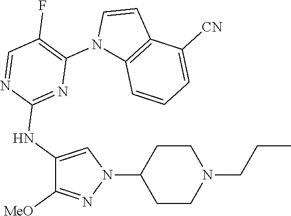 [SMILES: CCCN1CCC(n2cc(Nc3ncc(F)c(-n4ccc5c(C#N)cccc54)n3)c(OC)n2)CC1] | 475.2 | 1H NMR (500 MHz, DMF-d7): δ 0.91 (t, J = 7.4 Hz, 3H), 1.52 (s, 2H), 2.04 (s, 6H), 2.32 (s, 2H), 2.99 (s, 2H), 3.89 (s, 3H), 4.01 (s, 1H), 7.03 (d, J = 3.6 Hz, 1H), 7.45 (s, 1H), 7.78 (d, J = 7.4 Hz, 1H), 7.90 (s, 1H), 8.17- 8.21 (m, 1H), 8.64 (d, J = 4.2 Hz, 1H). |
| 11 | 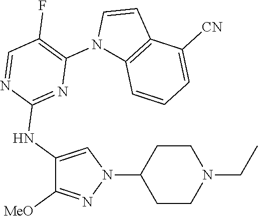 [SMILES: CCN1CCC(n2cc(Nc3ncc(F)c(-n4ccc5c(C#N)cccc54)n3)c(OC)n2)CC1] | 461.2 | 1H NMR (500 MHz, DMF-d7): δ 1.05 (t, J = 7.2 Hz, 3H), 1.93-2.09 (m, 6H), 2.38 (q, J = 7.2 Hz, 2H), 3.01 (d, J = 10.4 Hz, 2H), 3.89 (s, 3H), 3.98 (dt, J = 10.8, 5.8 Hz, 1H), 7.02 (d, J = 3.7 Hz, 1H), 7.45 (d, J = 8.4 Hz, 1H), 7.79 (d, J = 7.4 Hz, 1H), 7.89 (s, 1H), 8.18 (dd, J = 3.6, 2.2 Hz, 1H), 8.63 (d, J = 4.3 Hz, 1H). |
| 12 | 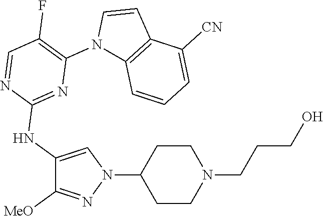 [SMILES: COc1nn(C2CCN(CCCO)CC2)cc1Nc1ncc(F)c(-n2ccc3c(C#N)cccc32)n1] | 491.3 | 1H NMR (500 MHz, DMF-d7): δ 1.99 (s, 2H), 2.29-2.49 (m, 4H), 3.00-3.27 (m, 4H), 3.64 (m, 4H), 3.90 (s, 3H), 4.39 (m, 1H), 7.03 (s, 1H), 7.48 (t, J = 8.0 Hz, 1H), 7.78 (d, J = 7.3 Hz, 1H), 7.95 (s, 1H), 8.19 (s, 1H), 8.64 (d, J = 4.2 Hz, 1H). |
| 13 | 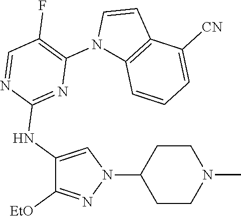 [SMILES: CCOc1nn(C2CCN(C)CC2)cc1Nc1ncc(F)c(-n2ccc3c(C#N)cccc32)n1] | 461.2 | 1H NMR (500 MHz, DMF-d7): δ 1.29 (t, J = 7.0 Hz, 3H), 1.91-2.14 (m, 6H), 2.23 (s, 3H), 2.88 (d, J = 11.3 Hz, 2H), 3.95 (m, 1H), 4.24 (q, J = 7.0 Hz, 2H), 7.03 (d, J = 3.6 Hz, 1H), 7.44 (s, 1H), 7.79 (d, J = 7.4 Hz, 1H), 7.90 (s, 1H), 8.18-8.2 (m, 1H), 8.64 (d, J = 4.2 Hz, 1H). |
| 14 | 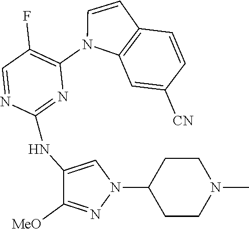 [SMILES: COc1nn(C2CCN(C)CC2)cc1Nc1ncc(F)c(-n2ccc3ccc(C#N)cc32)n1] | 447.3 | 1H NMR (500 MHz, DMF-d7): δ 1.95-2.07 (m, 6H), 2.22 (s, 3H), 2.87 (d, J = 10.7 Hz, 2H), 3.90 (s, 3H), 3.97 (s, 1H), 7.03 (d, J = 3.6 Hz, 1H), 7.63 (dd, J = 8.1, 1.4 Hz, 1H), 7.91 (d, J = 8.1 Hz, 2H), 8.18 (s, 1H), 8.62 (d, J = 4.4 Hz, 1H). |
| 15 | 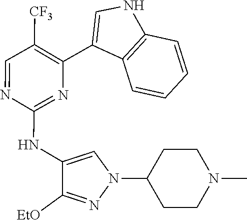 [SMILES: CCOc1nn(C2CCN(C)CC2)cc1Nc1ncc(C(F)(F)F)c(-c2c[nH]c3ccccc23)n1] | 486.2 | 1H NMR (500 MHz, DMF-d7): δ 1.29 (t, J = 7.0 Hz, 3H), 1.90-2.05 (m, 6H), 2.22 (s, 3H), 2.87 (d, J = 2.8 Hz, 2H), 4.01 (m, 1H), 4.24 (q, J = 7.0 Hz, 2H), 7.02 (s, 1H), 7.12 (s, 1H), 7.94 (s, 1H), 8.26 (s, 1H), 8.47 (s, 1H), 8.57 (s, 1H). |
| 16 | 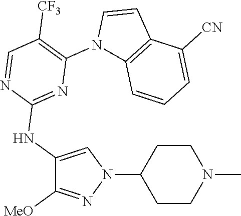 [SMILES: COc1nn(C2CCN(C)CC2)cc1Nc1ncc(C(F)(F)F)c(-n2ccc3c(C#N)cccc32)n1] | 497.1 | 1H NMR (500 MHz, DMF-d7): δ 2.19 (s, 2H), 2.40 (s, 4H), 2.95-3.17 (m, 6H), 3.77 (bs, 1H), 3.83-3.94 (m, 6H), 4.23 (m, 1H), 4.48 (m, 1H), 7.41 (t, J = 7.8 Hz, 2H), 7.66 (d, J = 7.3 Hz, 2H), 7.98 (d, J = 8.2 Hz, 2H), 8.04 (m, 2H), 8.77 (s, 2H), 9.20 (bs, 1H), 9.27 (bs, 1H). |
| 17 | 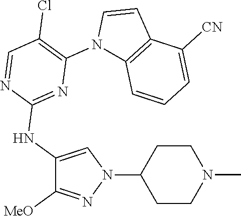 [SMILES: COc1nn(C2CCN(C)CC2)cc1Nc1ncc(Cl)c(-n2ccc3c(C#N)cccc32)n1] | 463.3 | 1H NMR (500 MHz, DMF-d7): δ 2.11 (s, 3H), 2.57 (m, 6H), 3.17 (bs, 2H), 3.89 (s, 3H), 4.08 (m, 1H), 7.42 (t, J = 7.8 Hz, 1H), 7.69 (d, J = 7.3 Hz, 1H), 7.96 (s, 1H), 7.97 (dd, J = 8.3, 1.0 Hz, 1H), 8.33 (s, 1H), 8.49 (s, 1H). |
| 18 | 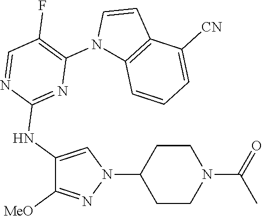 [SMILES: COc1nn(C2CCN(C(C)=O)CC2)cc1Nc1ncc(F)c(-n2ccc3c(C#N)cccc32)n1] | 475.3 | 1H NMR (500 MHz, DMF-d7): δ 1.80 (qd, J = 12.3, 4.5 Hz, 1H), 1.94 (qd, J = 12.2, 11.5, 3.8 Hz, 1H), 2.05 (m, 2H), 2.10 (s, 3H), 2.72- 2.8 (m, 1H), 3.27 (td, J = 13.8, 13.1, 2.8 Hz, 1H), 3.89 (s, 3H), 4.03 (d, J = 13.9 Hz, 1H), 4.30 (tt, J = 11.3, 4.1 Hz, 1H), 4.58 (d, J = 13.2 Hz, 1H), 7.02 (d, J = 3.6 Hz, 1H), 7.45 (d, J = 7.6 Hz, 1H), 7.78 (d, J = 7.4 Hz, 1H), 7.93 (s, 1H), 8.17-8.2 (m, 1H), 8.64 (d, J = 4.2 Hz, 1H). |
| 19 | 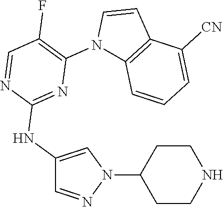 [SMILES: N#Cc1cccc2c1ccn2-c1nc(Nc2cnn(C3CCNCC3)c2)ncc1F] | 403.1 | 1H NMR: (400 MHz DMSO-d6): δ 9.84 (s, 1H), 8.71 (s, 2H), 8.40 (s, 1H), 8.12 (s, 1H), 8.11 (s, 1H), 7.79 (d, J = 8.0 Hz, 1H), 7.57 (s, 1H), 7.49-7.45 (m, 1H), 6.99 (s, 1H), 4.44 (s, 1H), 3.41-3.38 (m, 2H), 3.09-3.03 (m, 2H), 2.17-2.04 (m, 4H). |
| 20 | 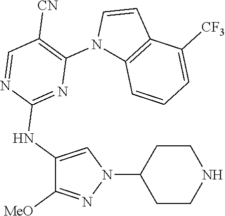 [SMILES: COc1nn(C2CCNCC2)cc1Nc1ncc(C#N)c(-n2ccc3c(C(F)(F)F)cccc32)n1] | 483.0 | 1H NMR (400 MHz DMSO-d6): δ 8.77 (s, 1H), 8.32-8.69 (dd, 1H), 8.21 (m, 1H), 7.76- 7.96 (ds, 1H), 7.60 (m, 1H), 7.35-7.47 (dt, 1H), 6.95 (br s, 1H), 4.26 (m, 1H), 3.94 (m, 3H), 3.50 (m, 3H), 3.15 (m, 2H), 2.19-2.31 (m, 4H). |
| 21 | 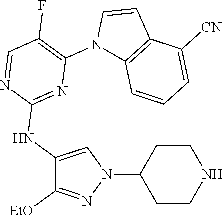 [SMILES: CCOc1nn(C2CCNCC2)cc1Nc1ncc(F)c(-n2ccc3c(C#N)cccc32)n1] | 447.1 | 1H NMR (400 MHz DMSO-d6): δ 8.81-8.79 (m, 2H), 8.61 (s, 1H), 8.46-8.44 (m, 1H), 8.10 (s, 1H), 7.76-7.74 (m, 2H), 7.35 (s, 1H), 6.96(d, J = 3.2 Hz, 1H), 4.31-4.29 (m, 1H), 4.29-4.27 (m, 2H), 3.39 (d, J = 8 Hz, 2H), 3.09-3.00 (m, 2H), 2.15-2.02 (m, 4H), 1.24 (s, 3H). |
| 22 | 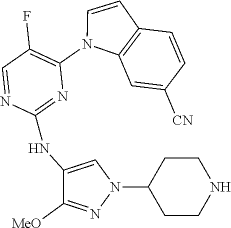 [SMILES: COc1nn(C2CCNCC2)cc1Nc1ncc(F)c(-n2ccc3ccc(C#N)cc32)n1] | 433.1 | 1H NMR (400 MHz DMSO-d6): δ 9.10 (brs, 1H), 8.73-8.72 (m, 1H), 8.60 (s, 1H), 8.43- 8.38 (m, 1H), 8.10 (s, 1H), 7.86-7.80 (m, 2H), 7.60 (d, J = 8.0 Hz, 1H), 6.97 (d, J = 4.0 Hz, 1H), 3.01 (d, J = 8.0 Hz, 1H), 4.32-4.27 (m, 1H), 3.84 (s, 3H), 3.39 (d, J = 12.0 Hz, 2H), 3.05-3.02 (m, 2H), 2.17-2.05 (m, 4H). |
| 23 | 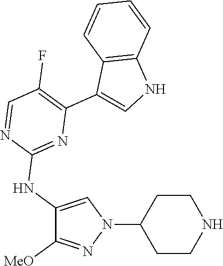 [SMILES: COc1nn(C2CCNCC2)cc1Nc1ncc(F)c(-c2c[nH]c3ccccc23)n1] | 408.1 | 1H NMR (400 MHz DMSO-d6): δ 11.8 (s, 1H), 8.27 (s, 2H), 8.26 (s, 1H), 7.72 (s, 1H), 7.46 (d, J = 8.0 Hz, 1H), 7.21-7.17 (m, 1H), 7.04 (s, 1H), 4.01-3.99 (m, 1H ), 3.77 (s, 3H), 3.03 (d, J = 12 Hz, 2H), 2.56-2.50 (m, 2H), 1.96-1.93 (m, 2H), 1.77-1.73 (m, 2H). |
| 24 | 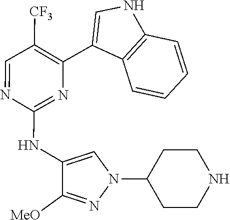 [SMILES: COc1nn(C2CCNCC2)cc1Nc1ncc(C(F)(F)F)c(-c2c[nH]c3ccccc23)n1] | 458.1 | 1H NMR (400 MHz DMSO-d6): δ 11.91 (s, 1H), 9.18-8.98 (m, 1H), 8.61-8.54 (m, 2H), 7.94-7.77 (m, 3H), 7.25 (s, 1H), 7.23 (s, 1H), 7.17-7.10 (m, 2H), 3.78 (s, 3H), 3.40-3.38 (m, 2H), 3.06-3.03 (m, 2H), 2.13-2.09 (m, 4H). |
| 25 | 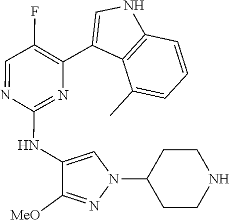 [SMILES: COc1nn(C2CCNCC2)cc1Nc1ncc(F)c(-c2c[nH]c3cccc(C)c23)n1] | 422.1 | 1H NMR (400 MHz DMSO-d6): δ 11.7 (s, 1H), 8.76-8.74 (m, 1H), 8.41-8.39 (m, 1H), 8.33(s, 2H), 7.74-7.72 (m, 2H), 7.30 (d, J = 8.0 Hz, 1H), 7.09-7.05 (m, 1H), 6.85 (d, J = 8.0 Hz, 1H ), 3.77 (s, 3H), 3.38 (d, J = 12.8 Hz, 2H), 3.04-3.01 (m, 2H), 2.38 (s, 3H), 2.12-2.00 (m, 4H). |
| 26 | 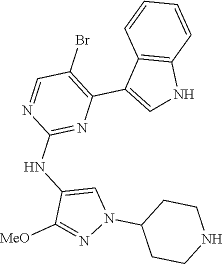 [SMILES: COc1nn(C2CCNCC2)cc1Nc1ncc(Br)c(-c2c[nH]c3ccccc23)n1] | 470.0 | 1H NMR (500 MHz DMSO-d6): δ 8.49 (s, 1H), 8.32 (s, 1H), 8.25 (br s, 2H), 7.68 (s, 1H), 7.43 (m, 1H), 7.25 (t, 1H), 7.05 (m, 1H), 3.95 (m, 1H), 3.77 (s, 3H), 3.38 (d, 2H), 3.02 (d, 2H), 2.55 (m, 2H), 1.93 (d, 2H), 1.72 (m, 2H). |
| 27 | 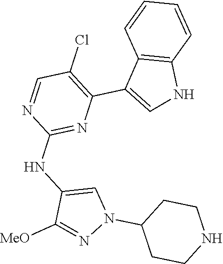 [SMILES: COc1nn(C2CCNCC2)cc1Nc1ncc(Cl)c(-c2c[nH]c3ccccc23)n1] | 424.1 | 1H NMR (400 MHz DMSO-d6): δ 11.80 (s, 1H), 8.20-8.70 (m, 4H), 7.70 (s, 1H), 7.37 (d, 1H), 7.12 (t, 1H), 6.95 (br s, 1H), 4.25 (m, 1H), 3.70 (s, 3H), 3.35 (d, 2H), 3.00 (q, 2H), 2.50 (s, 2H), 2.05 (m, 4H). |
| 28 | 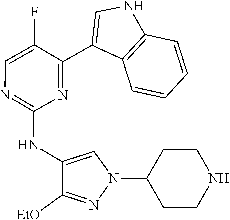 [SMILES: CCOc1nn(C2CCNCC2)cc1Nc1ncc(F)c(-c2c[nH]c3ccccc23)n1] | 422.1 | 1H NMR (300 MHz, MeOD-d4): δ ppm 8.38 (dd, J = 8.01, 2.92 Hz, 1 H), 8.10 (d, J = 3.01 Hz, 1 H), 8.04 (d, J = 4.52 Hz, 1 H), 7.69 (s, 1 H), 7.38 (d, J = 8.10 Hz, 1 H), 7.11-7.24 (m, 1 H), 6.93-7.09 (m, 1 H), 4.21-4.34 (m, 1 H), 4.05-4.20 (m, 2 H), 3.43-3.61 (m, 2 H), 3.05-3.16 (m, 2 H), 2.08-2.37 (m, 4 H), 1.22 (t, J = 6.97 Hz, 3H) |
| 29 | 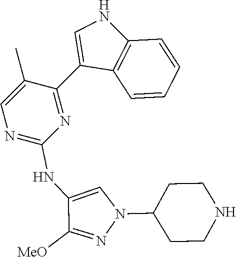 [SMILES: COc1nn(C2CCNCC2)cc1Nc1ncc(C)c(-c2c[nH]c3ccccc23)n1] | 404.0 | 1H NMR (300 MHz, MeOD-d4): δ 8.31 (d, 1H), 8.13 (s, 1H), 7.82 (d, 2H), 7.47 (d, 1H), 7.09-7.48 (m, 3H), 3.90-4.05 (m, 4H), 3.35 (m, 2H), 3.13 (d, 2H), 2.69 (t, 2H), 2.38 (s, 3H), 1.76-2.07 (m, 4H). |
| 30 | 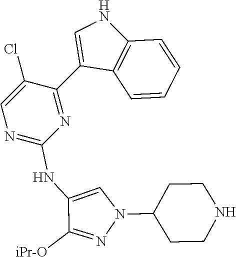 | 452.1 | 1H NMR (400 MHz DMSO-d6): δ 11.82 (s, 1H), 8.49 (s, 1H), 8.27-8.31 (m, 2H), 7.69 (s, 1H), 7.45 (d, 1H), 7.18 (t, 1H), 7.01 (br s, 1H), 4.74 (m, 1H), 3.96 (m, 1H), 3.01 (d, 2H), 2.56 (t, 2H), 1.92 (m, 2H), 1.73 (m, 2H), 1.20 (m, 6H). |
| 31 | 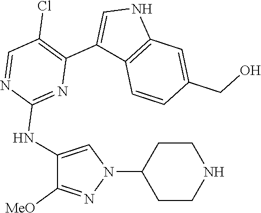 [SMILES: COc1nn(C2CCNCC2)cc1Nc1ncc(Cl)c(-c2c[nH]c3cc(CO)ccc23)n1] | 454.0 | 1H NMR (400 MHz, DMSO-d6) δ ppm 1.62- 1.85 (m, 2 H) 1.94 (d, J = 11.12 Hz, 2 H) 2.53-2.62 (m, 2 H) 3.03 (d, J = 12.63 Hz, 2 H) 3.78 (s, 3 H) 3.87-4.10 (m, 1 H) 4.52- 4.69 (m, 2 H) 5.15 (br. s., 1 H) 6.99 (br. s., 1 H) 7.41 (s, 1 H) 7.71 (s, 1 H) 8.30 (s, 1 H) 8.35 (br. s., 1 H) 8.43-8.52 (m, 1 H) 11.78 (br. s., 1 H) |
| 32 | | 461.3 | 1H NMR: (400 MHz DMSO-d6): δ 8.82 (br |
| | | | s, 1H), 8.77 (s, 1H), 8.09 (s, 1H), 7.74 (m, |
| | | | 2H), 7.34 (br s, 1H), 6.96 (s, 1H), 4.17 (m, |
| | | | 1H), 3.79 (s, 3H), 2.80 (m, 2H), 1.75-1.87 |
| | | | (dd, 2H), 1.51-1.61 (m, 3H), 1.10 (s, 3H), |
| | | | 1.06 (s, 3H). |
| 33 | 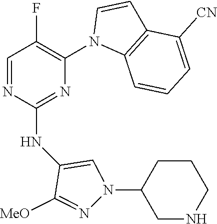 [SMILES: COc1nn(C2CCCNC2)cc1Nc1ncc(F)c(-n2ccc3c(C#N)cccc32)n1] | 433.1 | 1H NMR: (400 MHz DMSO-d6): δ 8.81 (br s, 1H), 8.60 (s, 1H), 8.09 (s, 1H), 7.75 (m, 2H), 7.33 (br s, 1H), 6.96 (s, 1H), 3.90 (m, 1H), 3.79 (s, 3H), 3.12 (d, 1H), 2.84 (d, 1H), 2.65 (t, 1H), 2.44 (t, 1H), 2.01 (m, 1H), 1.70- 1.81 (m, 2H), 1.45 (m, 1H). |
| 34 | 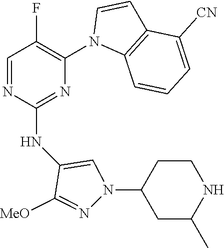 [SMILES: COc1nn(C2CCNC(C)C2)cc1Nc1ncc(F)c(-n2ccc3c(C#N)cccc32)n1] | 447.2 | 1H NMR: (400 MHz DMSO-d6): δ 8.56 (m, 3H), 8.07 (m, 1H), 7.70 (m, 2H), 7.35 (t, 1H), 6.94 (m, 1H), 3.99 (m, 1H), 3.82 (s, 3H), 3.05 (m, 2H), 2.66 (m, 2H), 1.93 (t, 2H), 1.68 (m, 1H), 1.36 (m, 1H), 1.05 (d, 3H). |
| 35 | 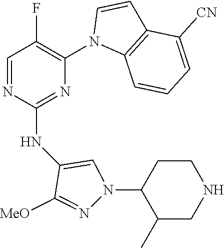 [SMILES: COc1nn(C2CCNCC2C)cc1Nc1ncc(F)c(-n2ccc3c(C#N)cccc32)n1] | 447.1 | 1H NMR: (400 MHz DMSO-d6): δ 8.55 (br s, 3H), 8.05 (s, 1H), 7.64-7.71 (m, 2H), 7.33 (m, 1H), 6.93 (br s, 1H), 4.16 (br s, 1H), 3.83 (br s, 3H), 3.15 (br s, 1H), 2.80 (m, 2H), 2.65 (m, 1H), 2.21 (br s, 1H), 1.97 (m, 1H), 1.74 (m, 1H), 0.71 (d, 3H). |

or a pharmaceutically acceptable salt thereof.

In one aspect, a composition is provided that includes an oligonucleotide and a compound of any one of Formulae I, Ia, Ib, Ic, or of Table 1, a pharmaceutically acceptable salt of a compound of any one of Formulae I, Ia, Ib, Ic, or of Table 1, or a combination thereof. In one aspect, a pharmaceutical composition is provided that includes an oligonucleotide; and a compound of any one of Formulae I, Ia, Ib, Ic, or of Table 1, a pharmaceutically acceptable salt of a compound of any one of Formulae I, Ia, Ib, Ic, or of Table 1, or a combination thereof, and a pharmaceutically acceptable diluent or carrier.

In one aspect, the oligonucleotide is single stranded. In one aspect, the oligonucleotide is double stranded. In one aspect, the oligonucleotide includes DNA. In one aspect, the oligonucleotide includes RNA. In one aspect, the oligonucleotide includes from about 8 to about 30 nucleotides. In one aspect, the oligonucleotide is an antisense oligonucleotide (ASO), a splice switching oligonucleotide (SSO), interfering RNA (RNAi), small interfering RNA (siRNA), micro RNA (miRNA), an antagomir, a decoy oligonucleotide, or a combination thereof.

In one aspect, the oligonucleotide includes one or more modified nucleotides. In one aspect, the one or more modified nucleotides comprise: phosphodiester (PO); phosphorothioate (PS); 2′O-methyl (2′OMe); 2′O-methoxyethyl (MOE); peptide nucleic acid (PNA); phosphoroamidate morpholino (PMO); locked nucleic acid (LNA); 2′-deoxy-2′-fluoro (2′-F); any other 2′ modified oligonucleotide; or a combination thereof.

In one aspect, a method of introducing an oligonucleotide into a nucleus and/or cytosol of a cell is provided. In one aspect, the method includes:
(a) contacting the cell with the oligonucleotide; and
(b) contacting the cell with a compound of any one of Formulae I, Ia, Ib, Ic, or of Table 1, a pharmaceutically acceptable salt of a compound of any one of Formulae I, Ia, Ib, Ic, or of Table 1, or a combination thereof. In one aspect, the compound facilitates entry of the oligonucleotide into the nucleus and/or cytosol of the cell. In one aspect, the oligonucleotide is internalized by the cell through endocytosis and encapsulated within an endosome and the compound facilitates release of the oligonucleotide from the endosome. In one aspect, the oligonucleotide is internalized by transient pore formation induced by the compound.

In one aspect, contacting the cell with the oligonucleotide of (a) is performed in a composition, wherein the composition of (a) includes about 0.025 μM to about 20 μM of the oligonucleotide. In one aspect, the composition of (a) includes about 0.1 μM to about 10 μM, about 0.1 μM to about 5 μM, or about 0.1 μM to about 1 μM of the oligonucleotide. In one aspect, the cell is contacted with a second composition that includes about 1 μM to about 10 μM of the compound.

In one aspect, contacting the cell with a compound of (b) is performed in a composition, wherein the composition of (b) includes about 1 μM to about 20 μM of the compound. In one aspect, the composition of (b) includes at least about 1 μM, about 1.5 μM, about 2 μM, about 2.5 μM, about 3 μM, about 3.5 μM, about 4 μM, about 4.5 μM, or about 5 μM and up to about 6 μM, about 6.5 μM, about 7 μM, about 7.5 μM, about 8 μM, about 8.5 μM, about 9 μM, about 9.5 μM, about 10 μM, about 10.5 μM, about 11 μM, about 11.5 μM, about 12 μM, about 12.5 μM, about 13 μM, about 13.5 μM, about 14 μM, about 14.5 μM, about 15 μM, about 15.5 μM, about 16 μM, about 16.5 μM, about 17 μM, about 17.5 μM, about 18 μM, about 18.5 μM, about 19 μM, about 19.5 μM, or about 20 μM, of the compound.

In one aspect, the cell is contacted with the oligonucleotide of (a) and the compound of (b) at approximately the same time.

In one aspect, the cell is contacted with the oligonucleotide of (a) before being contacted with a compound of (b). In one aspect, the cell is contacted with the oligonucleotide of (a) up to about 48 hours before being contacted with the compound of (b). In one aspect, the cell is contacted with the oligonucleotide of (a) about 12 hours to about 48 hours before being contacted with the compound of (b).

In one aspect, the contacting the cell with the oligonucleotide of (a) and the contacting the cell with a compound of (b) are performed in the same composition. In one aspect, the composition includes about 0.025 μM to about 10 μM of the oligonucleotide. In one aspect, the composition includes about 0.1 μM to about 5 μM, or about 0.1 μM to about 1 μM of the oligonucleotide. In one aspect, the composition includes about 1 μM to about 20 μM of the compound. In one aspect, the composition includes about 1 μM to about 10 μM of the compound. In one aspect, the composition includes about 1 μM, about 1.5 μM, about 2 μM, about 2.5 μM, about 3 μM, about 3.5 μM, about 4 μM, about 4.5 μM, or about 5 μM and up to about 6 μM, about 6.5 μM, about 7 μM, about 7.5 μM, about 8 μM, about 8.5 μM, about 9 μM, about 9.5 μM, about 10 μM, about 10.5 μM, about 11 μM, about 11.5 μM, about 12 μM, about 12.5 μM, about 13 μM, about 13.5 μM, about 14 μM, about 14.5 μM, about 15 μM, about 15.5 μM, about 16 μM, about 16.5 μM, about 17 μM, about 17.5 μM, about 18 μM, about 18.5 μM, about 19 μM, about 19.5 μM, or about 20 μM of the compound.

In one aspect, the oligonucleotide hybridizes to a target nucleic acid in the cell. In one aspect, the target nucleic acid is in the nucleus of the cell. In one aspect, the target nucleic acid is in the cytosol of the cell.

In one aspect, contacting the cell with the compound of (b) results in endosomal membrane permeabilization as determined by mCherry-GAL9 recruitment assay and with reduced accumulation of the oligonucleotide of the oligonucleotide in endosomes and/or lysosomes.

In one aspect, the oligonucleotide alters activity of a gene expressed by the cell. In one aspect, the oligonucleotide increases activity of a gene expressed by the cell. In one aspect, the activity of the gene expressed by the cell is increased at least about 10× when the cell is contacted with the oligonucleotide and the compound as compared to a cell that is contacted with the oligonucleotide and not the compound. In one aspect, the activity of the gene expressed by the cell is increased at least about 100× when the cell is contacted with the oligonucleotide and the compound as compared to a cell that is contacted with the oligonucleotide and not the compound.

In one aspect, the oligonucleotide decreases activity of a gene expressed by the cell. In one aspect, the activity of the gene expressed by the cell is decreased at least about 10× when the cell is contacted with the oligonucleotide and the compound as compared to a cell that is contacted with the oligonucleotide and not the compound. In one aspect, the activity of the gene expressed by the cell is decreased at least about 100× when the cell is contacted with the oligonucleotide and the compound as compared to a cell that is contacted with the oligonucleotide and not the compound.

In one aspect, the method includes in vitro delivery of the oligonucleotide to the cell. In one aspect, the method includes in vivo delivery of the oligonucleotide to the cell.

In one aspect, the cell is a cultured cell. In one aspect, the cell is an isolated cell. In one aspect, the cell is an isolated cell from a subject in need of treatment. In one aspect, the cell is part of a tissue or organ. In one aspect, the organ or tissue is the brain, central nervous system (CNS) or peripheral nervous system (PNS), heart, liver, kidney, spleen, pancreas, lung, adipose, and/or muscle (e.g., skeletal muscle). In one aspect, the cell is a brain cell, a CNS cell, a PNS cell, a heart cell, a liver cell, a kidney cell, a spleen cell, a pancreas cell, a lung cell, a muscle cell, an adipose cell, an immune cell, or combination thereof.

In one aspect, the cell is a mammalian cell. In another aspect, the cell is a eukaryotic cell and/or a prokaryotic cell.

In one aspect, a method of altering expression of a target nucleic acid in a cell is provided. In one aspect, the method that includes contacting the cell with an oligonucleotide that is capable of hybridizing to the target nucleic acid, wherein the oligonucleotide is internalized by the cell through endocytosis and encapsulated within an endosome; and contacting the cell with a compound of any one of Formulae I, Ia, Ib, or Ic, or of Table 1, a pharmaceutically acceptable salt of a compound of any one of Formulae I, Ia, Ib, or Ic, or of Table 1, or a combination thereof, wherein the compound facilitates release of the oligonucleotide from the endosome, wherein hybridization of the oligonucleotide to the target nucleic acid alters expression of the target nucleic acid. In one aspect, hybridization of the oligonucleotide to the target nucleic acid increases expression of the target nucleic acid. In one aspect, hybridization of the oligonucleotide to the target nucleic acid decreases expression of the target nucleic acid. In one aspect, hybridization of the oligonucleotide to the target nucleic acid alters splicing of the target nucleic acid.

In one aspect, a method of releasing an oligonucleotide from an endosome is provided. In one aspect, the method includes: contacting the cell with the oligonucleotide, wherein the oligonucleotide is internalized by the cell through endocytosis and encapsulated within the endosome; and contacting the cell with a compound of any one of Formulae I, Ia, Ib, or Ic, or of Table 1, a pharmaceutically acceptable salt of a compound of any one of Formulae I, Ia, Ib, or Ic, or of Table 1, or a combination thereof, wherein the compound facilitates release of the oligonucleotide from the endosome.

In one aspect, a method for the treatment and/or prevention of a disorder in a subject is provided. In one aspect, the method includes: administering to the subject a therapeutically effective amount of an oligonucleotide and an effective amount of a compound of any one of Formulae I, Ia, Ib, or Ic, or of Table 1, or a combination thereof. In one aspect, the compound is administered to the subject concurrently with the oligonucleotide. In one aspect, the compound is administered to the subject after administration of the oligonucleotide. In one aspect, the compound is administered to the subject up to about 48 hours after administration of the oligonucleotide. In one aspect, the compound is administered to the subject about 12 hours to about 48 hours after administration of the oligonucleotide. In one aspect, administration includes parenteral administration. In one aspect, administration includes intravenous or subcutaneous administration. In one aspect, the subject is a mammal. In one aspect, the subject is a human.

In one aspect, a method for the treatment and/or prevention of a disorder in a subject is provided that includes: isolating a cell from the subject; contacting the isolated cell with a therapeutically effective amount of an oligonucleotide and an effective amount of a compound of any one of Formulae I, Ia, Ib, or Ic, or of Table 1, a pharmaceutically acceptable salt of a compound of any one of Formulae I, Ia, Ib, or Ic, or of Table 1, or a combination thereof to produce an engineered cell; and transplanting the engineered cell in the subject. In one aspect, the isolated cell is contacted with a composition that includes about 0.025 μM to about 20 μM of the oligonucleotide. In one aspect, the composition includes about 0.1 μM to about 10 μM, about 0.1 μM to about 5 μM, or about 0.1 μM to about 1 μM of the oligonucleotide. In one aspect, the isolated cell is contacted with a composition that includes about 1 μM to about 20 μM of the compound. In one aspect, the composition includes about 1 μM to about 10 μM of the compound. In one aspect, the composition includes at least about 1 μM, about 1.5 μM, about 2 μM, about 2.5 μM, about 3 μM, about 3.5 μM, about 4 μM, about 4.5 μM, or about 5 μM and up to about 6 μM, about 6.5 μM, about 7 μM, about 7.5 μM, about 8 μM, about 8.5 μM, about 9 μM, about 9.5 μM, about 10 μM, about 10.5 μM, about 11 μM, about 11.5 μM, about 12 μM, about 12.5 μM, about 13 μM, about 13.5 μM, about 14 μM, about 14.5 μM, about 15 μM, about 15.5 μM, about 16 μM, about 16.5 μM, about 17 μM, about 17.5 μM, about 18 μM, about 18.5 μM, about 19 μM, about 19.5 μM, or about 20 μM of the compound. In one aspect, the isolated cell is contacted with the oligonucleotide and the compound at approximately the same time. In one aspect, the isolated cell with a composition that includes the oligonucleotide and the compound. In one aspect, the isolated cell is contacted with the oligonucleotide before the isolated cell is contacted with the compound. In one aspect, the isolated cell is contacted with the oligonucleotide up to about 48 hours before the isolated cell is contacted with the compound. In one aspect, the isolated cell is contacted with the oligonucleotide about 12 hours to about 48 hours after the isolated cell is contacted with the compound. In one aspect, the subject is a mammal. In one aspect, the subject is a human.

In one aspect, use of a compound of any one of Formulae I, Ia, Ib, or Ic, or of Table 1, a pharmaceutically acceptable salt of a compound of any one of Formulae I, Ia, Ib, or Ic, or of Table 1, or a combination thereof is provided for the manufacture of a medicament for gene therapy.

In one aspect, a kit is provided that includes a compound of any one of Formulae I, Ia, Ib, or Ic, or of Table 1, a pharmaceutically acceptable salt of a compound of any one of Formulae I, Ia, Ib, or Ic, or of Table 1, or a combination thereof and an oligonucleotide.

### BRIEF DESCRIPTION OF THE FIGURES

FIG. 1A shows the chemical structure for UNC2383, a small molecule endosomolytic compound described by Wang et al. (2018) ACS Chem. Biol. 12(8):1999-2007.

FIG. 1B shows the chemical structure of Compound 2 (also referred to as AZ4800), described herein.

FIG. 1C shows the chemical structure of Compound 6 (also referred to as AZ2467 or A7), described herein.

FIG. 2A is a graph showing functional activity of a splice switching oligonucleotide (SSO) with co-treatment with UNC2383 and other select compounds including AZ4800 (Compound 2). HeLaLuc-705 cells were pre-treated with 1 μM SSO, and then co-treated with 10 μM, 5 μM, or 1 μM of UNC2383 or other select compounds including AZ4800 (Compound 2) for 2 hours. Cells were then rinsed and incubated for 4 hours in growth media. Values are reported as fold increase over 1 μM naked SSO.

FIG. 2B is a graph showing the increase in functional SSO activity when co-treated with select compounds including AZ4800 (Compound 2) and AZ2467 (Compound 6). HeLaLuc-705 cells were exposed to the same co-treatment plan as above, with 5 μM of compound and either 1 μM SSO or 100 nM SSO.

FIG. 3 are images showing Gal9 body-inducing endosomal rupture of HeLa mCherry-Gal9 cells treated with UNC2383 or other select compounds including AZ2467 (Compound 6) and AZ4800 (Compound 2), across a 10-point dose titration series for 2 hours and then stained for nuclei (Hoechst 33342). Representative images display the relocalisation of Gal9 from cytosolic to punctate in a dose-dependent manner. Scale bar=20 μm.

FIG. 4 are images showing Gal-9 body-inducing endosomal rupture of Huh7 mCherry-Gal9 cells treated with UNC2383, or other select compounds including AZ2467 (Compound 6) and AZ4800 (Compound 2) across 10-point dose titration series for 2 hours and then stained for nuclei (Hoechst 33342). Representative images display the relocalisation of Gal9 from cytosolic to punctate in a dose-dependent manner. Scale bar=20 μm.

FIG. 5 are graphs showing the narrow window in concentration between induction of endosomal rupture and cytotoxicity for HeLa and Huh7 cells treated with DMSO, UNC2383 or other select compounds including AZ2467 (Compound 6) and AZ4800 (Compound 2). Gal9 punctate reported as a percentage of max quantitation, normalized to nuclei signal. Cell survival reported as a percentage of all cells.

FIG. 6 are images showing Huh7mCherry-Gal9 stable cells treated for up to 4 hours with 5 μM of select compounds including SMC5/AZ4800 (Compound 2), A7 (Compound 6), A1 (Compound 5) and DMSO in a Gal9-recruitment assay.

FIG. 7 is a graph showing quantitation of Gal9 recruitment in Huh7 cells treated with UNC2383, or other select compounds including SMC5/AZ4800 (Compound 2), A7 (Compound 6), A1 (Compound 5) and DMSO, revealing a time-dependent endosomal rupture, even from compounds which did not exhibit detectable functional activity. Values are reported as puncta per relative nuclear signal and plotted normalized to the highest overall induced signal (SMC5/AZ4800 (Compound 2), 60 min, Huh7).

FIG. 8 is a graph showing is a graph showing quantitation of Gal9 recruitment in HeLa cells treated with select compounds including SMC5/AZ4800 (Compound 2), A7 (Compound 6), A1 (Compound 5) and DMSO, revealing a time-dependent endosomal rupture, even from compounds which did not exhibit detectable functional activity. Values are reported as puncta per relative nuclear signal, and plotted normalized to the highest overall induced signal (SMC5/AZ4800 (Compound 2), 60 min, Huh7)

FIG. 9 shows the results of RT-PCR with gel analysis to detect aberrant or functional mRNA copies in cells treated with AZ4800 (Compound 2) and AZ2467 (Compound 6). Gel images were quantitated using Quantity One (BioRad) software. Values of functional mRNA were reported as a percentage of total mRNA.

FIG. 10 is a schematic of treatment plan in which cells are seeded in growth media containing DMEM+10% FBS with added SSO. After 24 hours, an endosomolytic compound diluted in growth media is added to the desired final concentration. After 2 hours of co-treatment, media is removed, cells are washed with phosphate buffered saline (PBS), and incubated in growth media for 4 hours.

FIG. 11A is a graph showing the increase in functional SSO activity obtained using different treatment plans with AZ4800 (Compound 2). The “positive” group underwent the treatment described in connection with FIG. 10. The “preload only” group was treated with 1 μM SSO for 24 hours and then 5 μM compound for 2 hours. The “no-preload” group was treated with 1 μM SSO and 5 μM AZ4800 (Compound 2) simultaneously for 2 hours. Values are reported as fold increase over untreated.

FIG. 11B is a graph showing the increase in functional SSO activity in HeLaLuc-705 cells co-treated with 1 μM SSO and various concentrations of AZ2467 (Compound 6). Co-treatment duration lasted 30 min, 1 hr, 2 hr, 4 hr, or 6 hr followed by the same 4 hr incubation step. Values are reported as fold increase over SSO only.

FIG. 11C is a graph showing the increase in functional SSO activity in HeLaLuc-705 cells co-treated with 1 μM SSO and various concentrations of AZ4800 (Compound 2). Co-treatment duration lasted 30 min, 1 hr, 2 hr, 4 hr, or 6 hr followed by the same 4 hr incubation step. Values are reported as fold increase over SSO only.

FIG. 12A is a graph showing the increase in efficacy of 705-SSO in HeLa cells when treated with differing concentrations of AZ4800 (Compound 2) following the treatment plan shown schematically in FIG. 10, with the exception that the pre-loading step was performed for 48 hours instead of 24. Cells were analyzed after 2, 4, and 6 hours of treatment with AZ4800 (Compound 2). Values are reported as fold increase over naked oligo-only treatment.

FIG. 12B is a graph showing the increase in efficacy of 705-SSO in Huh7 cells when treated with differing concentrations of AZ4800 (Compound 2) following the treatment plan shown schematically in FIG. 10, with the exception that the pre-loading step was performed for 48 hours instead of 24. Cells were analyzed after 2, 4, and 6 hours of treatment with AZ4800 (Compound 2). Values are reported as fold increase over naked oligo-only treatment.

FIG. 13A is a graph showing the increase efficacy of 705-SSO in HeLa cells when treated with differing concentrations of AZ4800 (Compound 2). The cells in the “pre-load” group were treated similarly to the treatment plan described in FIG. 10, while cells in the “no pre-load” group were only co-treated with AZ4800 (Compound 2) and 1 μM 705-SSO. Values are reported as fold increase over naked oligo-only treatment.

FIG. 13B is a graph showing the increase efficacy of 705-SSO in Huh7 cells when treated with differing concentrations of AZ4800 (Compound 2). The cells in the “pre-load” group were treated similarly to the treatment plan described in FIG. 10, while cells in the “no pre-load” group were only co-treated with AZ4800 (Compound 2) and 1 μM 705-SSO. Values are reported as fold increase over naked oligo-only treatment.

FIG. 14A and FIG. 14B are graphs showing the results of a nanoparticle tracking analysis, revealing no significant changes in particle size profile when cell growth media containing 1 μm (FIG. 14A) or 5 μm (FIG. 14B) oligonucleotide is incubated with 5 μm AZ4800 (Compound 2) for 2 hours. Composite profiles are generated from 5×30-second videos of each condition.

FIG. 15A are confocal microscopy images of a live-cell mCherry-Gal9 assay with 488-SSO in HeLa cells showing Gal9 relocalization and simultaneous decrease of SSO-containing bodies. Huh7Gal9-mCherry cells were pre-loaded with 488-SSO for 24 hours and then treated with various concentrations of AZ4800 (Compound 2) and Hoechst to visualize nuclei. Images were acquired every 10 minutes. Scale bars=20 μM.

FIG. 15B are confocal microscopy images of a live-cell mCherry-Gal9 assay with 488-SSO in Huh7 cells showing Gal9 relocalization and simultaneous decrease of SSO-containing bodies. Huh7Gal9-mCherry cells were pre-loaded with 488-SSO for 24 hours and then treated with various concentrations of AZ4800 (Compound 2) and Hoechst to visualize nuclei. Images were acquired every 10 minutes. Scale bars=20 μM.

FIG. 16A is a graph showing the quantitative analysis of the images in FIG. 15A, showing a time and dose-dependent decrease in the total number of 488-SSO puncta, normalized to total nuclear signal.

FIG. 16B is a graph showing the quantitative analysis of the images in FIG. 15B, showing a time and dose-dependent decrease in the total number of 488-SSO puncta, normalized to total nuclear signal.

FIG. 17A is a graph showing the quantitative analysis of Gal9-mCherry puncta in HeLa cells, showing a dose and time dependent increase of Gal9 bodies, normalized to nuclear signal.

FIG. 17B is a graph showing the quantitative analysis of Gal9-mCherry puncta in Huh7 cells, showing a dose and time dependent increase of Gal9 bodies, normalized to nuclear signal.

FIG. 18 is a schematic of the synthesis of Compound 2.

FIG. 19 is a schematic of the synthesis of Compound 3.

FIG. 20 is a schematic of the synthesis of Compound 4.

FIG. 21 is a schematic of the synthesis of Compound 7.

FIG. 22 is a schematic of the synthesis of Intermediate A.

FIG. 23A is a graph showing the increase efficacy of Luc 705-SSO in HeLa cells when treated with select compounds including AZ3325 (Compound 7) and AZ3327 (Compound 4).

FIG. 23B is a graph showing the increase efficacy of 705-SSO in HeLa cells with higher concentrations of AZ3327 (Compound 4).

FIG. 24 is a graph showing the increase efficacy of Luc 705-SSO in HeLa cells when treated with select compounds including AZ2862 (Compound 8) and AZ3327 (Compound 4).

FIG. 25A is a graph showing the increase efficacy of Luc 705-SSO in U2OS cells when treated with select compounds including AZ4374 (Compound 21), AZ4376 (Compound 22), AZ2862 (Compound 8).

FIG. 25B is a graph showing the increase efficacy of Luc 705-SSO in N2A cells when treated with select compounds including AZ4374 (Compound 21), AZ4376 (Compound 22), AZ2862 (Compound 8).

FIG. 26 is a graph showing the increase efficacy of Luc 705-SSO in HeLa cells with select compounds including AZ4800 (Compound 2), AZ3327 (Compound 4), AZ4374 (Compound 21), AZ2862 (Compound 8) and AZ3325 (Compound 7).

FIG. 27 is a graph comparing enhanced oligo activity of AZ3327 (Compound 4) within three cell types (HeLa_Luc705, U2OS_Luc705, and N2A_Luc705 cells) vs oligo alone.

FIG. 28 is a graph comparing enhanced oligo activity of AZ3327 (Compound 4) vs oligo alone in enhancing knockdown of MALAT1 mRNA expression.

FIG. 29 is a general schematic of the synthesis of the preparation of Compounds 2-7, 19-31.

FIG. 30 is a breakdown of the starting material “X” identified in FIG. 29 and the required intermediate for each of the Compounds 2-7, 19-31.

FIG. 31 is a general schematic of the synthesis of the preparation of Compounds 8-17 with the additional alkylation step.

FIG. 32 is a general schematic of the synthesis of the preparation of Compound 18.

FIG. 33 is a graph showing the GAL9 response results associated with select compounds at various doses (1.25-5 μM) in Huh7 cells.

FIG. 34 is a graph showing the GAL9 response results associated with select compounds at various doses (1.25-5 μM) in Hela cells.

FIG. 35 illustrates images showing Gal9 body-inducing endosomal rupture of HeLa mCherry-Gal9 cells treated with select compounds.

FIG. 36 illustrates images showing Gal9 body-inducing endosomal rupture of HeLa mCherry-Gal9 cells treated with select compounds.

FIG. 37 illustrates images showing Gal9 body-inducing endosomal rupture of HeLa mCherry-Gal9 cells treated with select compounds.

FIG. 38 illustrates images showing Gal9 body-inducing endosomal rupture of HeLa mCherry-Gal9 cells treated with select compounds.

FIG. 39 illustrates images showing Gal9 body-inducing endosomal rupture of HeLa mCherry-Gal9 cells treated with select compounds at various doses (0.3125-10 μM).

FIG. 40 is a graph comparing time (0.25 to 4 hours), dose of select compounds (0.31 to 10 μM) and GAL9 response in Huh7 cells.

FIG. 41 is a graph comparing varying dose (0.31 to 10 μM) and GAL9 response between different cell lines at 2 hours post-dosing with select compounds.

FIG. 42 includes a graph and a Table comparing administration of (i) unconjugated siRNA (“Duplex-PPIB”); (ii) siRNA conjugated with cholesterol (“Chol-PPIB”); (iii) siRNA conjugated GalNAc (“GalNAc-PPIB”); and (iv) unconjugated siRNA co-administered with 2 μM Compound 32 (“Duplex-PPIB+ERE”).

FIG. 43A illustrates a legend for the data shown in FIGS. 43B and 43C. Total cell counts are shown as unfilled bars (left panel); viable cells are shown in light gray shaded bars (middle panel); edited cell counts are shown in green shaded bars, and edited cell percentage is shown as the dotted line (right panel). FIG. 43B includes a graph showing T-47D cells co-administered with 50 nM Cre protein and varying concentrations of select compound, with either media changed after 2 hours (left panel) or unchanged (right panel). FIG. 43C includes a graph showing HeLa cells co-administered with 50 nM Cre protein and varying concentrations of select compound, with either media changed after 2 hours (left panel) or unchanged (right panel).

FIG. 44A shows immunofluorescence staining of coronal and sagittal brain segments of mice co-administered with Cre recombinase and varying concentrations of select compound. FIG. 44B shows immunofluorescence staining of lumbar spinal cord segments of mice co-administered with Cre recombinase and varying concentrations of select compound. FIG. 44C illustrates a dissection strategy and immunofluorescence results of each dissection segment. FIG. 44D shows IVIS® in vivo imaging and immunofluorescence staining of the pre-frontal, pre-injection, and injection site dissection segments.

FIG. 45A includes a graph showing percent viability and percent ex vivo editing (as measured by % tdTomato fluorescence) in non-activated T cells. FIG. 45B includes a graph showing percent viability and percent ex vivo editing in pre-activated T cells. FIG. 45C includes a graph showing percent viability and percent ex vivo editing in post-activated T cells.

FIG. 46A shows a schematic for the editing-based GFP reporter system. FIG. 46B includes a graph showing HEK cells co-administered with 50 nM Cas base editor and varying concentrations of select compound. FIG. 46C includes a graph showing HEK and N2a cells co-administered with 2.5 μM select compound and varying concentrations of base editor.

FIG. 47 includes images showing Gal9 body-inducing endosomal rupture of Huh7 mCherry-Gal9 cells treated with select compounds at various doses (0.3125-10 μM).

FIG. 48 shows a graph comparing varying dose (0.31 to 10 μM) and GAL9 response between different cell lines at 2 hours post-dosing with select compounds.

FIG. 49 is a graph showing the increase in functional SSO activity in HeLaLuc-705 cells co-treated with 1 μM SSO and various concentrations of AZ5219 (Compound 32). Co-treatment duration lasted 30 min, 1 hr, 2 hr, 4 hr, or 6 hr followed by the same 4 hr incubation step. Values are reported as fold increase over SSO only.

### DETAILED DESCRIPTION OF THE DISCLOSURE

### A. Definitions

Unless otherwise defined, scientific and technical terms used herein shall have the meanings that are commonly understood by those of ordinary skill in the art. Further, unless otherwise required by context, singular terms shall include pluralities and plural terms shall include the singular, for example, “a” or “an,” include pluralities, e.g., “one or more” or “at least one” and the term “or” can mean “and/or” unless stated otherwise. The terms “including,” “includes” and “included” are not limiting. Ranges provided herein, of any type, include all values within a particular range described and values about an endpoint for a particular range.

“Alkyl” refers to a saturated, branched or straight-chain hydrocarbon group. “Lower alkyl” refers to an alkyl group having from 1 and up to about 8 carbon atoms, for example, 1, 2, 3, 4, 5, 6, 7, or 8 carbon atoms. In one aspect, the alkyl group includes from 1 to 4 carbon atoms (C₁-C₄ alkyl). Lower alkyls include, but are not limited to, methyl, ethyl, propyl, isopropyl, and butyl, including n-butyl, sec-butyl, isobutyl, and tert-butyl.

The alkyl group may be substituted or unsubstituted. In some embodiments, the alkyl group is substituted with one or more halo groups, e.g., F, Cl, Br, I, At, etc. In some embodiments, the alkyl group is substituted with one or more F or Cl, e.g., a mono-, di- or tri-fluoro or chloro alkyl. An alkyl group in which one or more of the hydrogen atoms are replaced by halogen can be referred to as “halo alkyl”, e.g., “halo C₁-C₄ alkyl” refers to a C₁-C₄ alkyl substituted by one or more of the same or different halogen atoms. Examples of C₁-C₄ alkyls substituted with one or more halo groups include, but are not limited to, trifluoromethyl, difluoromethyl, fluoromethyl, trichloromethyl, dichloromethyl, and chloromethyl.

“Alkoxy,” also represented as “—OR₄” where R₄ is an alkyl group, refers to a saturated or unsaturated branched or straight-chain hydrocarbon group attached to a parent molecule through an oxygen atom. In one aspect, the alkoxy group includes from 1 to 4 carbon atoms (C₁-C₄ alkoxy). Examples of C₁-C₄ alkoxy include, but are not limited to, methoxy, ethoxy, propoxy, isopropoxy, butoxy, including n-butoxy, sec-butoxy iso-butoxy and t-butoxy. In some embodiments, the alkoxy group is substituted with one or more halo groups, e.g., F, Cl, Br, I, At, etc. In some embodiments, the alkoxy group is substituted with one or more F or Cl, e.g., a mono-, di-, or tri-fluoro or chloro alkoxy.

“Halogen” or “halo” can be used interchangeably to refer to a fluoro, chloro, bromo, or iodo group. In one aspect, halo refers to fluoro, chloro, or bromo. In one aspect, halo refers to fluoro or chloro.

“Amide” refers to the group “—C(O)NR₅R₅” (also represented as “—C(═O)NR₅R₅”) where each R₅ is independently hydro or alkyl (optionally substituted and/or interrupted) and includes primary, secondary, and tertiary amides. In one aspect, the alkyl in the amide includes a C₁ to C₈ alkyl. In one aspect, the alkyl substituent includes a C₁ to C₄ alkyl. In some embodiments, at least one R₅ in the formula “—C(O)NR₅R₅” is a hydro.

“Ester” refers to the group “—C(O)OR₆” group (also represented as (“—C(═O)OR₆”) where R₆ is hydro or alkyl. In some embodiments, the ester is a “short chain ester,” wherein R₆ is C₁-C₄ alkyl. In some embodiments, R₆ is methyl or ethyl. In some embodiments, R₆ is propyl or isopropyl.

“Cyano” refers to a group that includes a carbon atom triple-bonded to a nitrogen atom (—C≡N).

“Hydro” refers to a hydrogen substituent and is also represented by “—H.”

“Endosomolytic” refers to a compound that facilitates release of an oligonucleotide from an endosome/lysosome/autophagosome/multivesicular body or other endosomal vesicle into the cytosol of a cell. In one aspect, the endosomolytic agent is a small molecule compound (SMC). In one aspect, the endosomolytic agent has a structure represented by any of Formulae I, Ia, or of Table 1, or a pharmaceutically acceptable salt thereof.

“Small molecule compound” or “SMC” refers to an organic molecule with a molecular weight of less than 1000 g/Mol and includes compounds having a structure represented by any of Formulae I, Ia, or of Table 1.

“Nucleic acid” refers to an oligomer or polymer of nucleotides and includes naturally occurring or synthetically produced single stranded or double stranded deoxyribonucleotides (DNA) or ribonucleotides (RNA). A nucleic acid can include naturally occurring nucleic acid nucleobases such as adenine (A), guanine (G), thymine (T), cytosine (C) and uracil (U), as well base analogs or modified nucleobases that do not occur in nature.

“Target nucleic acid” refers to a nucleic acid to which an antisense oligonucleotide hybridizes. In one aspect, hybridization of an antisense oligonucleotide to a target nucleic acid in a cell alters activity of a gene expressed by the cell. In one aspect, hybridization of the antisense oligonucleotide to the target nucleic acid increases activity of a gene expressed by the cell. In one aspect, hybridization of the antisense oligonucleotide to the target nucleic acid decreases activity of a gene expressed by the cell.

“Oligonucleotide” refers to an exogeneous, naturally occurring, or non-naturally occurring single-stranded or double-stranded polymer of deoxyribonucleotides (DNA) or ribonucleotides (RNA). In one aspect, the oligonucleotide is about 2 to about 50 nucleotides in length. In one aspect, the oligonucleotide includes one or more nucleotide analogs or modified backbone residues or linkages, including, but not limited to, phosphodiester (PO); phosphorothioate (PS); 2′O-methyl (2′OMe); 2′O-methoxyethyl (MOE); peptide nucleic acid (PNA); phosphoroamidate morpholino (PMO); locked nucleic acid (LNA); 2′-deoxy-2′-fluoro (2′-F); or a combination thereof. “Locked nucleic acid nucleoside” or “LNA” refers a nucleoside that includes a bicyclic sugar moiety with a 4′-CH₂—O-2′bridge. “Phosphorothioate” refers to an internucleotide linkage in which one of the non-bridging oxygens is replaced by sulfur.

As “modified oligonucleotide” refers to an oligonucleotide that includes at least one modified nucleoside and/or at least one modified internucleoside linkage.

An “antisense oligonucleotide” or “ASO” is an oligonucleotide that includes at least a portion of which is complementary to a target nucleic acid such that the ASO can hybridize to the target nucleic acid. An antisense oligonucleotide can increase or decrease expression of a target nucleic acid.

A “splice switching oligonucleotide” or “SSO” is a short, synthetic, antisense oligonucleotide that can hybridize to a pre-mRNA and disrupt splicing of the transcript, for example, by blocking the RNA-RNA base pairing or protein-RNA binding interactions that occur between components of the splicing machinery and the pre-mRNA. “Pre-mRNA” refers to an RNA transcript that includes one or more introns and has not been fully processed into mRNA.

A “small interfering RNA,” also known as “short interfering RNA,” “silencing RNA,” or “siRNA,” is a class of double-stranded RNA that is non-coding, typically between about 20 to about 25 base pairs, with hydroxylated 3′ and phosphorylated 5′ ends. In general, siRNA is part of the RNA interference pathway and interferes with expression of specific genes with complementary nucleotide sequences by degrading mRNA after transcription, thereby preventing translation. siRNA may be conjugated e.g., to sugars such as GalNAc or lipids such as cholesterol, to enhance delivery to a target cell, e.g., with improved pharmacokinetics and/or efficacy. See, e.g., Osborn et al., Nucleic Acid Ther. 28(3):128-136 (2018). In some aspects, the present disclosure provides a method of enhancing delivery of unconjugated siRNA.

“Polypeptide,” used interchangeably herein with “peptide” or “protein,” refers to a polymeric form of amino acids of any length, which can include coded and non-coded amino acids, chemically or biochemically modified or derivatized amino acids, and polypeptides having modified peptide backbones. In some aspects, a polypeptide comprises about 2 to about 5000 amino acids. In some aspects, the polypeptide is capable of providing a site-specific modification in a target nucleic acid. In some embodiments, the polypeptide is a therapeutic polypeptide.

A “macromolecule” includes proteins, nucleic acids, carbohydrates, lipids, nanogels, and macrocycles. In some aspects, a macromolecule of the present disclosure comprises an oligonucleotide. In some aspects, a macromolecule of the present disclosure comprises a polypeptide. In some aspects, a macromolecule of the present disclosure comprises one or more components of a site-specific modification (SSM) system described herein, e.g., a CRISPR system, a Cre-Lox system, and/or a FLP-FRT system. Throughout the present disclosure, references to a SSM system can mean any one or more components of the system. In some aspects, a SSM system comprises a CRISPR system, a Cre-Lox system, a FLP-FRT system, any component thereof, or any combination thereof.

A “CRISPR” system is a SSM system capable of performing an SSM at a target nucleic acid. In some aspects, a CRISPR system includes (a) a protein capable of providing the SSM, e.g., a Cas protein; and (b) a guide RNA (also referred to herein as “gRNA”), which includes (i) a “crRNA” or “spacer” region that hybridizes to the target nucleic acid, and (ii) a “tracrRNA” or “scaffold” region that associates with the protein. In some aspects, the SSM comprises single-stranded cleavage of the target nucleic acid. In some aspects, the SSM comprises double-stranded cleavage of the target nucleic acid. In some aspects, the SSM comprises a deletion. In some aspects, the SSM comprises an insertion. In some aspects, the SSM comprises a mutation. In some aspects, the SSM comprises a base edit, e.g., conversion of a C-G base pair to a T-A base pair.

A “Cre-Lox” system is a SSM system capable of performing an SSM at a target nucleic acid. In some aspects, a Cre/Lox system includes a Cre recombinase, which recognizes a pair of Lox (also called LoxP) sequences flanking the target nucleic acid and catalyzes site-specific recombination at the target nucleic acid. An analogous system to the Cre-Lox system is the “FLP-FRT” system. A FLP-FRT system is a SSM system capable of performing an SSM at a target nucleic acid. In some aspects, a FLP-FRT system includes the FLP recombinase, which recognizes a pair of FRT sequences flanking the target nucleic acid and catalyzes site-specific recombination at the target nucleic acid. In some aspects, the SSM comprises an inversion. In some aspects, the SSM comprises an insertion. In some aspects, the SSM comprises a deletion. In some aspects, the SSM comprises a translocation. In some aspects, the location and orientation of the Lox sequences (or the FRT sequences) determines the type of SSM (e.g., inversion, deletion, or translocation) performed by the Cre recombinase (or the FLP recombinase).

“Hybridize” refers to the pairing of complementary oligomeric compounds, for example, pairing between an antisense oligonucleotide and its corresponding target nucleic acid. While not limited to any mechanism, the most common mechanism of pairing involves hydrogen bonding between complementary nucleobases, including, for example, Watson-Crick, Hoogsteen, or reversed Hoogsteen hydrogen bonding. For example, in Watson-Crick base pairing, guanine (G) is complementary to cytosine (C), adenine (A) is complementary to thymine (T) in DNA, and adenine (A) is complementary to uracil (U) in RNA. Additionally, some modified nucleobases maintain the ability to pair with a counterpart nucleobase. Hybridization can occur between two complementary DNA molecules (DNA-DNA hybridization), two RNA molecules (RNA-RNA hybridization), or between complementary DNA and RNA molecules (DNA-RNA hybridization). Hybridization can occur between a short nucleotide sequence that is complementary to a portion of a longer nucleotide sequence. Hybridization can occur between sequences that do not have 100% “sequence complementarity,” i.e., complementary sequences need not have nucleobase complementarity at each nucleoside, although sequences having less sequence complementarity are less stable and less likely hybridize than sequences having greater sequence complementarity.

“Specifically hybridizes” refers to the ability of an oligonucleotide to hybridize to a target nucleic acid with greater affinity than to a different nucleic acid. In one aspect, the antisense oligonucleotide specifically hybridizes to a target nucleic acid sequence under physiological conditions, for example, for in vivo or therapeutic use.

“Targeting” or “targeted to,” in the context of antisense oligonucleotides, refers to the association of an antisense oligonucleotide with a particular target nucleic acid or region of a target nucleic acid. An antisense oligonucleotide targets a target nucleic acid if it is sufficiently complementary to the target nucleic acid to allow hybridization under physiological conditions. “Targeting” or “targeted to,” in the context of SSM systems (e.g., CRISPR, Cre-Lox, and/or FLP-FRT systems described herein), refers to the association of the protein of the SSM system (e.g., Cas protein, Cre recombinase, or FLP recombinase) with a particular target nucleic acid or region of a target nucleic acid. In some aspects, the Cas protein of a CRISPR system targets a target nucleic acid upon hybridization of the guide RNA with the target nucleic acid. In some embodiments, the Cre recombinase of a Cre-Lox system targets a target nucleic acid upon recognition of the Lox sequences flanking the target nucleic acid. In some embodiments, the FLP recombinase of a FLP-FRT system targets a target nucleic acid upon recognition of the FRT sequences flanking the target nucleic acid.

“Alter” or “modulate” refer a change in an amount, function, or activity of a molecule, e.g., a macromolecule described herein, when compared to the amount, function, or activity prior to treatment. In one aspect, a compound described herein increases or decreases an amount, function or activity of a gene expressed by a target nucleic acid sequence. In one aspect, the compound increases the activity of an antisense oligonucleotide (ASO) that acts on pre-mRNA via RNase H in the nucleus. In one aspect the compound increases the activity of an siRNA that acts via the RISC complex in the cytosol. In one aspect, the compound increases the alteration of pre-mRNA splicing by a splice switching oligonucleotide (SSO), as reflected by an increase in the desired splice variant. In one aspect, the compound results in reduced levels of the corresponding target mRNA and/or protein as compared to treatment with the ASO in the absence of the compound. In some aspects, the compound increases the activity of a Cas protein in a CRISPR system. In some aspects, the compound increases the activity of a Cre recombinase. In some aspects, the compound increases the activity of a FLP recombinase. In some aspects, the compound increases the frequency of the SSM at the target nucleic acid. In some aspects, the compound increases the editing efficiency of a SSM system (e.g., the CRISPR, Cre-Lox, and/or FLP-FRT systems).

In one aspect, the compound described herein “enhances the delivery” of a macromolecule provided herein, e.g., an oligonucleotide and/or polypeptide. In one aspect, the compound described herein “enhances the delivery” of an antisense oligonucleotide to increase cytosolic and/or nuclear concentration, accumulation, and/or half-life of the oligonucleotide as compared to that found without administration of the compound. In one aspect, the compound described herein “enhances the delivery” of one or more components of a SSM system to increase cytosolic and/or nuclear concentration, accumulation, and/or half-life of the SSM system as compared to that found without administration of the compound. In some aspects, the SSM system comprises a CRISPR system, a Cre-Lox system, a FLP-FRT system, any component thereof, or any combination thereof.

“Expression” refers to a process by which a protein is produced in a host cell from a nucleic acid and includes, but is not limited to, transcription, translation, post-translational modification, and secretion. “Increased” expression is in the context of a comparison between a treated cell and an untreated control, for example, a cell treated with a macromolecule, e.g., an oligonucleotide such as an antisense oligonucleotide, a polypeptide such as a recombinase (e.g., Cre or FLP), or a combination thereof such as a Cas protein and a guide RNA, versus an untreated cell, or a cell treated with a macromolecule, e.g., an oligonucleotide such as an antisense oligonucleotide, a polypeptide such as a recombinase (e.g., Cre or FLP), or a combination thereof such as a Cas protein and a guide RNA, and a compound described herein versus a cell treated with only the macromolecule, e.g., the antisense oligonucleotide, recombinase (e.g., Cre or FLP), or Cas protein and guide RNA. Similarly, “decreased” expression is in the context of a comparison between a treated cell and an untreated control, for example, a cell treated with a macromolecule, e.g., an oligonucleotide such as an antisense oligonucleotide, a polypeptide such as a recombinase (e.g., Cre or FLP), or a combination thereof such as a Cas protein and a guide RNA, versus an untreated cell, or a cell treated with a macromolecule, e.g., an oligonucleotide such as an antisense oligonucleotide, a polypeptide such as a recombinase (e.g., Cre or FLP), or a combination thereof such as a Cas protein and a guide RNA, and a compound described herein versus a cell treated with only the macromolecule, e.g., the antisense oligonucleotide, recombinase (e.g., Cre or FLP), or Cas protein and guide RNA.

“Disease” refers to any disease, disorder, condition, symptom, or indication.

“Treating” or “treatment” refer to curative, symptomatic, preventive and prophylactic treatment and include, but are not limited to, arresting or ameliorating a disease or at least one clinical symptoms of a disease, reducing the risk of acquiring a disease or at least one clinical symptoms of a disease, reducing the development of a disease or at least one clinical symptoms of the disease, reducing the risk of developing a disease or at least one clinical symptoms of a disease, or delaying the onset of the disease or at least one clinical symptoms of a disease.

A “subject” and “patient” can be used interchangeably to refer to any animal subjects, for example, mammalian subjects such as humans, primates, cattle, pigs, horses, sheep, goats, rodents, cats, and/or dogs. In one aspect, the subject is human.

The term “cell” can include a single cell, a plurality of cells or a population of cells where context permits, unless otherwise specified. In one aspect, the cell is in vitro, for example, a cell explanted from a subject. In one aspect, the cell is a cell grown in batch culture or in tissue culture. In one aspect, the cell is in vivo, for example, located in a subject in need of treatment. In one aspect, the subject is a human subject.

“Pharmaceutically acceptable” as used herein means approved by a regulatory agency of a Federal or state government, or listed in the U.S. Pharmacopeia, European Pharmacopia or other generally recognized pharmacopeia for use in animals, and more particularly in humans.

A “pharmaceutical composition” includes one or more active agents, including, for example, a macromolecule, e.g., an oligonucleotide such as an antisense oligonucleotide, a polypeptide such as a recombinase (e.g., Cre or FLP), or a combination thereof such as a Cas protein and a guide RNA, and a compound described herein, and a pharmaceutically acceptable carrier or diluent. In one aspect, the carrier or diluent is suitable for intravenous, intramuscular, subcutaneous, parenteral, spinal or epidermal administration.

“Pharmaceutically acceptable salt” refers to a salt of a compound that is physiologically and pharmaceutically acceptable and that possesses the desired pharmacological activity of the parent compound and includes a salt prepared from pharmaceutically acceptable non-toxic acid or base, including inorganic or organic acids and bases. “Pharmaceutically acceptable salts” of the compounds described herein may be prepared by methods well-known in the art. For a review of pharmaceutically acceptable salts, see Stahl and Wermuth, Handbook of Pharmaceutical Salts: Properties, Selection and Use (Wiley-VCH, Weinheim, Germany, 2002).

An “effective amount” of a compound refers to an amount sufficient to increase the efficacy of a macromolecule, e.g., an oligonucleotide such as an antisense oligonucleotide, a polypeptide such as a recombinase (e.g., Cre or FLP), or a combination thereof such as a Cas protein and a guide RNA. In one aspect, an “effective” amount of a compound refers to an amount sufficient to facilitate entry of a macromolecule, e.g., an oligonucleotide such as an antisense oligonucleotide, a polypeptide such as a recombinase (e.g., Cre or FLP), or a combination thereof such as a Cas protein and a guide RNA, into the nucleus and/or cytosol of a cell. In one aspect, an “effective” amount of a compound refers to an amount that facilitates the release of a macromolecule, e.g., an oligonucleotide such as an antisense oligonucleotide, a polypeptide such as a recombinase (e.g., Cre or FLP), or a combination thereof such as a Cas protein and a guide RNA, from an endosome into the cytosol of a cell.

A “therapeutically effective amount” of a macromolecule, e.g., an oligonucleotide such as an antisense oligonucleotide, a polypeptide such as a recombinase (e.g., Cre or FLP), or a combination thereof such as a Cas protein and a guide RNA, refers to an amount sufficient to provide a therapeutic benefit in the treatment of a disease, or to delay or reduce one or more symptoms associated with the disease. A “therapeutically effective amount” can vary depending on many factors, including, but not limited to, the macromolecule, e.g., an oligonucleotide such as an antisense oligonucleotide, a polypeptide such as a recombinase (e.g., Cre or FLP), or a combination thereof such as a Cas protein and a guide RNA, being administered, the disease, the severity of the disease, the age of the subject being treated, and/or the weight of the subject being treated.

“Dose” refers to a specified quantity of an active agent (for example, a macromolecule, e.g., an oligonucleotide such as an antisense oligonucleotide, a polypeptide such as a recombinase (e.g., Cre or FLP), or a combination thereof such as a Cas protein and a guide RNA; and a compound) provided in a single administration, or in a specified time-period. A dose can be administered in one, two, or more boluses or injections. In one aspect, the active agent is administered by infusion over an extended period of time or continuously. Doses can be stated as the amount of pharmaceutical agent per unit time (e.g., hour, day, week, or month). Doses can also be stated as the amount per unit weight of the subject (e.g., mg/kg or g/kg).

“Dosage unit” refers to a form in which an active agent, for example, a macromolecule, e.g., an oligonucleotide such as an antisense oligonucleotide, a polypeptide such as a recombinase (e.g., Cre or FLP), or a combination thereof such as a Cas protein and a guide RNA; and a compound, is provided. In one aspect, the dosage unit is a vial containing lyophilized active agent. In one aspect, a dosage unit is a vial containing reconstituted active agent. In one aspect, the active agent is a macromolecule. In one aspect, the active agent is an oligonucleotide. In one aspect, the active agent comprises a polypeptide. In one aspect, the active agent comprises an antisense oligonucleotide. In one aspect, the active agent comprises a Cre recombinase. In one aspect, the active agent comprises a FLP recombinase. In one aspect, the active agent comprises Cas protein. In one aspect, the active agent comprises Cas protein and a guide RNA. In one aspect, the active agent comprises a CRISPR system, a Cre-Lox system, a FLP-FRT system, any component thereof, or any combination thereof.

The methods and compositions described herein can be used in vitro on a sample (for example, on isolated cells, organs, or tissues) or in vivo in a subject (for example, in a living organism, such as a patient).

The compositions described herein may be administered in a number of ways depending upon whether local or systemic treatment is desired and the area to be treated. In one aspect, the composition is administered parenterally. Parenteral administration includes, but is not limited to, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, and intrasternal injection and infusion. Administration can be continuous, chronic, short, or intermittent.

In one aspect, a compound described herein and a macromolecule described herein such as, e.g., an oligonucleotide, e.g., antisense oligonucleotide, siRNA, or guide RNA, and/or a polypeptide, e.g., a recombinase (e.g., Cre or FLP) or a Cas protein, are administered concurrently. As used herein, “concurrently” refers to co-administration of the compound and the macromolecule (e.g., oligonucleotide and/or polypeptide, e.g., antisense oligonucleotide, siRNA, recombinase (e.g., Cre or FLP), or one or both of a Cas protein and guide RNA) sufficiently close in time to produce a combined effect. Concurrent administration does not require that the compound and the macromolecule (e.g., oligonucleotide and/or polypeptide, e.g., antisense oligonucleotide, siRNA, recombinase (e.g., Cre or FLP), or one or both of a Cas protein and guide RNA) be administered in a single pharmaceutical composition, in the same dosage form, or by the same route of administration. “Concurrently” includes simultaneous administration, or sequential administration within a short period of time, for example, in which the compound and the macromolecule (e.g., oligonucleotide and/or polypeptide, e.g., antisense oligonucleotide, siRNA, recombinase (e.g., Cre or FLP), or one or both of a Cas protein and guide RNA) are administered within about 6 hours, about 3 hours, about 1 hour, or about 30 minutes of each other and includes administering the compound before, simultaneously with, or after the macromolecule (e.g., oligonucleotide and/or polypeptide, e.g., antisense oligonucleotide, siRNA, recombinase (e.g., Cre or FLP), or one or both of a Cas protein and guide RNA).

In one aspect, the compound and the macromolecule (e.g., oligonucleotide and/or polypeptide, e.g., antisense oligonucleotide, siRNA, recombinase (e.g., Cre or FLP), or one or both of a Cas protein and guide RNA) are administered sequentially. As used herein, “sequential” administration means that the compound and the macromolecule (e.g., oligonucleotide and/or polypeptide, e.g., antisense oligonucleotide, siRNA, recombinase (e.g., Cre or FLP), or one or both of a Cas protein and guide RNA) are administered to the patient or cell at a different point in time. In one aspect, the compound is administered to the patient or cell at least about 12 hours or 24 hours and up to 36 hours or 48 hours after administration of the macromolecule (e.g., oligonucleotide and/or polypeptide, e.g., antisense oligonucleotide, siRNA, recombinase (e.g., Cre or FLP), or one or both of a Cas protein and guide RNA).

### B. Overview

Delivering macromolecules such as oligonucleotides or proteins into cells requires that the macromolecule, e.g., oligonucleotide or protein, traverse cellular membranes, including the plasma membrane and/or endosomal membranes. Use of macromolecules such as oligonucleotides or proteins can be hindered by their inability to effectively reach the cytosolic and/or nucleus of the cell, for example, due to their inability to cross the cell membrane or to escape from endosomal compartments following endocytosis. Provided herein are compounds that can increase the activity of a macromolecule, e.g., an oligonucleotide, such as an antisense oligonucleotide or siRNA; a polypeptide, such as recombinase (e.g., Cre or FLP); or a combination thereof, such as a Cas protein and a guide RNA. In one aspect, the compound is a small molecule compound (SMC). In one aspect, the compound is an endosomolytic compound that facilitates the release of the macromolecule, e.g., oligonucleotide and/or polypeptide from an endosome into the cytosol of a cell. In one aspect, the compound disclosed herein increase transfection efficiency of a macromolecule, e.g., an oligonucleotide and/or polypeptide. Advantageously, the compounds described herein interfere with the normal cell trafficking machinery to a minimal extent, i.e., until leakage is induced and do not induce damage or toxicity which irreversibly impedes cell proliferation or results in cell death.

### C. Oligonucleotides

In one aspect, compositions and methods are provided for delivering a macromolecule, e.g., an oligonucleotide, to the cytosol and/or nucleus of a cell. In one aspect, the oligonucleotide is single stranded. In one aspect, the oligonucleotide is double stranded. In one aspect, the oligonucleotide includes deoxyribonucleic acid (DNA). In one aspect, the oligonucleotide includes ribonucleic acid (RNA). In one aspect, the oligonucleotide is about 5 nucleotides to about 100 nucleotides, about 5 nucleotides to about 50 nucleotides, about 8 nucleotides to about 30 nucleotides, about 10 nucleotides to about 30 nucleotides, about 15 nucleotides to about 30 nucleotides, or about 18 to about 30 nucleotides in length. In one aspect, the oligonucleotide has a molecular weight from about 5 kDa to about 15 kDa.

In one aspect, the oligonucleotide reduces expression of a target nucleic acid, which can be referred to as “gene silencing.” In one aspect, the oligonucleotide increases expression of a target nucleic acid, which can be referred to as “gene activation.” In one aspect, the oligonucleotide alters the splicing of a target nucleic acid, which can be referred to as “splice switching.” In one aspect, the oligonucleotide interacts with a target protein. In one aspect, the oligonucleotide is an agonist or antagonist of a target protein.

In one aspect, the oligonucleotide is an antisense oligonucleotide (ASO), a small (˜18-30 nucleotides), synthetic, single-stranded nucleic acid polymer which modulates gene expression via various mechanisms. In one aspect, the ASO includes DNA and forms an RNA-DNA heteroduplex that is recognized by endogenous RNase H enzyme which catalyzes the degradation of RNA, thereby decreasing expression of the gene. In one aspect, the ASO binds to a target nucleic acid but does not induce degradation. In one aspect, the oligonucleotide is a splice switching oligonucleotide (SSO) that masks sequences within a target nucleic acid and thereby interferes with transcript RNA-RNA and/or RNA-protein interactions. In one aspect, the oligonucleotide is a small-interfering RNA (siRNA), which has a characteristic 19+2mer structure (e.g., a duplex of two 21-nucleotide RNA molecules with 19 complementary bases and terminal 2-nucleotide 3′ overhangs). In one aspect, the oligonucleotide is a microRNA (miRNA). In one aspect, the oligonucleotide targets a non-coding RNA sequence associated with transcriptional repression to reverse the effects of this negative regulation thereby activating gene expression. Other oligonucleotides include, but are not limited to, interfering RNA (RNAi) and decoy oligonucleotides. In one aspect, the oligonucleotide is a gapmer. In one aspect, the oligonucleotide is an aptamer.

In some aspects, the oligonucleotide is part of a site-specific modification (SSM) system, e.g., a CRISPR system, described herein. In some aspects, the oligonucleotide is a guide RNA. In one aspect, the guide RNA comprises one or both of: (i) a scaffold region or tracrRNA capable of associating with a Cas protein (e.g., Cas nuclease); and (ii) a spacer region or crRNA capable of hybridizing to a specific target nucleic acid sequence, thereby directing the Cas protein to make a site-specific modification at the target nucleic acid. In general, the spacer region is about 15 to about 25 nucleotides in length. In some aspects, the guide RNA is a single guide RNA (sgRNA) comprising both the tracrRNA and the crRNA. In some aspects, the guide RNA comprises a tracrRNA and a crRNA as two separate oligonucleotides that, together with the Cas protein, are capable of forming a complex.

In one aspect, the oligonucleotide includes one or more modified nucleotides. In one aspect, the oligonucleotide includes one or more modifications to the oligonucleotide phosphate linkages. In one aspect, the oligonucleotide includes one or more modifications to the ribose sugar. In one aspect, the oligonucleotide includes one or more nucleotides that are covalently modified to limit conformation, i.e., locked nucleic acids (LNA). In one aspect, the oligonucleotide includes a peptide nucleic acid (PNA). In one aspect, the oligonucleotide includes a methylated cystosine at the 5′ position. In one aspect, the oligonucleotide includes one or more modified nucleotides selected from: phosphodiester (PO); phosphorothioate (PS); 2′O-methyl (2′OMe); 2′O-methoxyethyl (MOE); peptide nucleic acid (PNA); phosphoroamidate morpholino (PMO); locked nucleic acid (LNA); 2′-deoxy-2′-fluoro (2′-F); or a combination thereof. In embodiments, the oligonucleotide is an antisense oligonucleotide targeting metastasis-associated lung adenocarcinoma transcript 1 (MALAT1). In embodiments, the oligonucleotide is an antisense oligonucleotide targeting metastasis-associated lung adenocarcinoma transcript 1 (MALAT1) comprising at least one nucleic acid with an LNA. In such embodiments, the antisense oligonucleotide targets MALAT1 and has the sequence: GM5CAttm5ctaatagm5cAGM5C, where m5c is 5-methylcytidine and capital letters are LNA nucleosides (SEQ ID NO:4). In embodiments, the oligonucleotide is an antisense oligonucleotide that targets MALAT1 and reduces expression of MALAT1 in cells by about 1% to about 100%. In embodiments, the oligonucleotide is an antisense oligonucleotide that targets MALAT1 and reduces expression of MALAT1 in cells by about 1%, by about 10%, by about 20%, by about 30%, by about 40%, by about 50%, by about 60%, by about 70%, by about 80%, by about 90%, by about 95% or by about 100%.

### D. Polypeptides

In one aspect, compositions and methods are provided for delivering a macromolecule, e.g., a polypeptide, to the cytosol and/or nucleus of a cell. In some aspects, the polypeptide comprises a therapeutic protein, which may be an antibody or a non-antibody protein. In one aspect, the polypeptide is a therapeutic peptide, e.g., as described in Wang et al., Sig Transduct Target Ther. 7:48 (2022).

In some aspects, the polypeptide is capable of providing a SSM at a target nucleic acid. In some aspects, the target nucleic acid is DNA. In some aspects, the target nucleic acid is RNA. In some aspects, the polypeptide is a nuclease. In some aspects, the polypeptide is a recombinase. In some aspects, the polypeptide is part of a SSM system described herein, e.g., a CRISPR system, a Cre-Lox system, or a FLP-FRT system. In some aspects, the polypeptide is a Cas protein. In some aspects, the polypeptide is Cas9. In some aspects, the polypeptide is Cas12a. In some aspects, the polypeptide is a recombinase. In some aspects, the polypeptide is Cre. In some aspects, the polypeptide is FLP.

In some aspects, the polypeptide comprises a modified Cas protein. In some aspects, the modified Cas protein is a Cas nickase, e.g., Cas9 nickase or Cas12a nickase, which cleaves only one strand of a double-stranded target nucleic acid. In some aspects, the modified Cas protein is a catalytically inactivated Cas protein (dead Cas), e.g., dCas9 or dCas12a, which does not comprise nuclease activity. In some aspects, the dead Cas is capable of binding to a target nucleic acid and preventing other enzymes such as transcription factors from binding to the target nucleic acid. Cas nickase and dead Cas proteins are further described, e.g., in Xu et al., J Mol Biol. 431(1):34-47 (2019); Qi et al., Cell 152(5):1173-1183 (2013); and Liu et al., Microbial Cell Factories 19:172 (2020).

In some aspects, the polypeptide comprises a Cas fusion protein. In some aspects, the Cas fusion protein comprises a modified Cas protein, e.g., a Cas nickase or a dead Cas, fused to an effector domain. In some aspects, the polypeptide comprises a Cas nickase or a dead Cas, e.g., dCas9 or dCas12, fused to a nucleotide deaminase, e.g., cytidine deaminase or adenosine deaminase, and optionally further fused to a DNA glycosylase inhibitor. In some aspects, the polypeptide comprises a Cas nickase, e.g., Cas9 nickase or Cas12 nickase, fused to a reverse transcriptase. Cas fusion proteins are further described, e.g., in Rees et al., Nat Rev Genet. 19(12):770-788 (2018); Anzalone et al., Nature 576(7785):149-157 (2019); and Liu et al., Microbial Cell Factories 19:172 (2020).

In some aspects, the polypeptide comprises a recombinase. In some aspects, the polypeptide comprises Cre. In some aspects, the polypeptide comprises FLP. In some aspects, the recombinase is a modified recombinase. In some aspects, the recombinase is an inducible recombinase. Modified (e.g., inducible) recombinases are further described, e.g., in Kaczmarcyk et al., Nucleic Acids Res. 29(12): e56 (2001); Badea et al., PLOS One 4(11): e7859 (2009); and Akbudak et al., Mol Biotechnol. 49(1):82-89 (2011).

### E. Cellular Entry

In some aspects, the macromolecule is delivered to a target cell. In one aspect, the target cell is a cultured cell. In one aspect, the target cell is an isolated cell. In one aspect, the target cell is an isolated cell from a subject in need of treatment. In one aspect, the target cell is a mammalian cell. In one aspect, the target cell is a eukaryotic cell. In one aspect, the target cell is a prokaryotic cell.

In one aspect, the target cell is part of a tissue or organ. In one aspect, the organ or tissue is the brain, central nervous system (CNS) or peripheral nervous system (PNS), heart, liver, kidney, spleen, pancreas, lung, adipose, and/or muscle (e.g., skeletal muscle). In one aspect, the target cell is a brain cell, a CNS cell, a PNS cell, a heart cell, a liver cell, a kidney cell, a spleen cell, a pancreas cell, a lung cell, a muscle cell, an adipose cell, an immune cell, or combination thereof.

In some aspects, the target cell is a CNS cell. In some aspects, the CNS cell comprises a glial cell and/or a neuron. Glial cells of the CNS include, e.g., astrocytes, oligodendrocytes, microglia, and ependymal cells. Neurons include, e.g., afferent neurons, efferent neurons, and interneurons.

In some aspects, the target cell is a liver cell, e.g., a hepatocyte or a non-parenchymal cell. In some aspects, the target cell comprises a plateable metabolism qualified human hepatocyte, a plateable induction qualified human hepatocyte, plateable human hepatocyte, suspension qualified human hepatocyte (including 10-donor and 20-donor pooled hepatocytes), human hepatic kupffer cells, human hepatic stellate cells, dog hepatocytes (including single and pooled Beagle hepatocytes), mouse hepatocytes (including CD-1 and C57BI/6 hepatocytes), rat hepatocytes (including Sprague-Dawley, Wistar Han, and Wistar hepatocytes), monkey hepatocytes (including Cynomolgus or Rhesus monkey hepatocytes), cat hepatocytes (including Domestic Shorthair hepatocytes), and rabbit hepatocytes (including New Zealand White hepatocytes).

In some aspects, the target cell is a stem cell, e.g., a human stem cell. The stem cells can be, for example, pluripotent stem cells, including embryonic stem cells (ESCs), adult stem cells, induced pluripotent stem cells (iPSCs), tissue specific stem cells (e.g., hematopoietic stem cells) and mesenchymal stem cells (MSCs). In some aspects, the cell is a differentiated form of any of the cells described herein. In some aspects, the eukaryotic cell is a cell derived from any primary cell in culture.

In some aspects, the target cell is an immune cell. Non-limiting examples of immune cells include T cells, B cells, dendritic cells, NK cells, T helper cells, cytotoxic T cells, regulatory T cells, gamma delta T cells, neutrophils, mast cells, monocytes, antigen-presenting cells, lymphocytes, basophils, and phagocytes.

In some aspects, the macromolecule to be delivered to a target cell is an oligonucleotide. In some aspects, the oligonucleotide is an antisense oligonucleotide. In some aspects, the oligonucleotide is a siRNA. In some aspects, the siRNA is unconjugated, i.e., not conjugated to a lipid or sugar. In some aspects, the siRNA is conjugated to a lipid and/or a sugar. In some aspects, the oligonucleotide is a guide RNA of a CRISPR system. In some aspects, the macromolecule to be delivered to a target cell is an polypeptide. In some aspects, the polypeptide is a Cas protein, e.g., Cas9 or Cas12a. In some aspects, the Cas protein is a modified Cas protein or a Cas fusion protein as described herein. In some aspects, the polypeptide is a recombinase. In some aspects, the polypeptide is Cre. In some aspects, the polypeptide is FLP. Oligonucleotides are hydrophilic polyanions with a molecular weight ranging from about 5 kDa to about 15 kDa and do not readily pass through the plasma membrane. Polypeptides also typically do not passively diffuse across the plasma membrane. To be effective, macromolecules such as oligonucleotides and polypeptides must traverse the plasma membrane and enter the cytosol and/or nucleus of the cell.

In one aspect, the macromolecule, e.g., oligonucleotide and/or polypeptide, is taken up by endocytosis, in which the macromolecule, e.g., oligonucleotide and/or polypeptide, is surrounded by the plasma membrane, which then buds off inside the cell to form a vesicle containing the ingested macromolecule, e.g., oligonucleotide and/or polypeptide. In another aspect, the macromolecule, e.g., oligonucleotide and/or polypeptide, is taken up by formation of pores in the plasma membrane.

The endocytic pathway of mammalian cells includes distinct membrane compartments which include: early endosomes, late endosomes and lysosomes.

Early endosomes (EE) are the first compartment of the endocytic pathway and are the main sorting station in the endocytic pathway (Huotari and Helenius (2011) EMBO J. 30(17):3481-3500). EEs recycle the majority of cargo internalized by endocytosis. Ees are heterogenous in terms of morphology, localization, composition, and function. Most Ees are relatively small and remain close to the plasma membrane, although the overall distribution of Ees is cell-type dependent.

Late endosomes (LE) receive endocytosed material, usually from early endosomes in the endocytic pathway and include proteins characteristic of nucleosomes, mitochondria and mRNAs including lysosomal membrane glycoproteins and acid hydrolases. They are acidic (40pprox. pH 5.5) and are thought to mediate a final sorting of the internalized cargo prior to delivery of the cargo to the lysosomes.

Lysosomes are compartment of the endocytic pathway which sequester cargo for arrest or degradation. Their chief function is to break down cellular waste products, fats, carbohydrates, proteins, and other macromolecules and return them to the cytoplasm as new cell-building materials. To accomplish this, lysosomes include a variety of hydrolytic enzymes that function in an acidic environment (40 approx. pH 4.8).

When taken up by endocytosis, macromolecules, e.g., oligonucleotides and/or polypeptides, often accumulate in endosomes, in particular, late endosomes or lysosomes, where they are pharmacologically inert. In order to be active, the macromolecule, e.g., oligonucleotide and/or polypeptide, must escape the endosomal compartment to access their cytosolic or nuclear targets before degradation or exportation via exocytosis.

### F. Compounds

Provided herein are compounds that facilitate entry of a macromolecule, e.g., an oligonucleotide and/or polypeptide, into the cytosol and/or nucleus of a cell. In one aspect, a small molecule compound (SMC) is provided that facilitates entry of the macromolecule, e.g., oligonucleotide and/or polypeptide, into the cytosol of a cell. In one aspect, the compound facilitates entry of the macromolecule, e.g., oligonucleotide and/or polypeptide, into the cytosol by forming pores in the plasma membrane of the cell. In one aspect, the compound facilitates release of the macromolecule, e.g., oligonucleotide and/or polypeptide, from an endosomal compartment within the cell. In one aspect, the macromolecule is an oligonucleotide, and the compound facilitates the endosomal escape of a gymnotically delivered oligonucleotide. In one aspect, the compound engorges the endosomal compartment, physically inducing membrane rupture and concurrent macromolecule, e.g., oligonucleotide and/or polypeptide, escape into the cytosol during the early-to-late endosomal transition and/or late endosomal transition to lysosome.

In one aspect, the compound has a structure represented by Formula I:

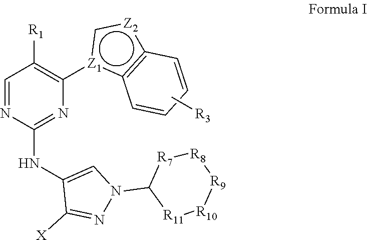

wherein
one of Z₁ and Z₂ is N, and the other is C;
R₁ is hydro, halo, C₁-C₄ alkyl, —OR₄, —C(═O)NR₅R₅, —CO₂R₆, or cyano;
X is hydro, C₁-C₄ alkyl, or —OR₂;
R₂ is hydro or C₁-C₄ alkyl;
R₃ is hydro, halo, C₁-C₄ alkyl, —(CH₂)_{y}OH, —OR₄, —C(═O)NR₅R₅, —CO₂R₆, or cyano;
R₄ is C₁-C₄ alkyl;
R₅ is hydro or C₁-C₄ alkyl;
R₆ is hydro or C₁-C₄ alkyl;
R₇, R₈, R₉, R₁₀, R₁₁ are each independently CHR₁₂, CR₁₂R₁₇ or NR₁₃;
R₁₂ is hydro, C₁-C₄ alkyl, —OR₁₄, or —CO₂R₁₅;
R₁₃ is hydro, C₁-C₄ alkyl, —(CH₂)_{y}OH, —OR₁₄, —CO₂R₁₅, or —C(═O)R₁₆;
R₁₄ is hydro or C₁-C₄ alkyl;
R₁₅ is hydro or C₁-C₄ alkyl;
R₁₆ is hydro or C₁-C₄ alkyl;
R₁₇ is hydro or C₁-C₄ alkyl;
y is 0, 1, 2, or 3; and
wherein one or more of the alkyl are optionally substituted with one or more halo, or a pharmaceutically acceptable salt thereof.

In one aspect, R₁ is hydro. In one aspect, R₁ is halo, including, for example, chloro, fluoro, bromo or iodo. In one aspect, R₁ is bromo, chloro, or fluoro. In one aspect, R₁ is fluoro. In one aspect, R₁ is alkyl. In one aspect, R₁ is a saturated alkyl. In one aspect, R₁ is an unsaturated alkyl, e.g., an alkenyl or alkynyl. In some aspects, R₁ can be polyunsaturated. In one aspect, R₁ is a straight chain alkyl. In one aspect, R₁ is a branched alkyl. In one aspect, R₁ is C₁-C₄ alkyl. In one aspect, R₁ is methyl, ethyl, propyl, isopropyl, or butyl. In one aspect, butyl includes n-butyl, sec-butyl, isobutyl, and tert-butyl. In one aspect, R₁ is a substituted alkyl, e.g., substituted with one or more halo. In some embodiments, R₁ is a C₁-C₄ alkyl, wherein at least one carbon of the alkyl is substituted with one or more chloro or fluoro. In some aspects, R₁ is substituted with a hydroxyl or a ketone. In one aspect, R₁ is an unsubstituted alkyl. In one aspect, R₁ is substituted with a halo group, i.e., a haloalkyl, e.g., a mono-haloalkyl, a di-haloalkyl, or a tri-haloalkyl. In one aspect, R₁ is a halo C₁-C₄ alkyl. In one aspect, R₁ is a halo C₁-C₄ alkyl such as trifluoromethyl, difluoromethyl, fluoromethyl, trichloromethyl, dichloromethyl, and chloromethyl.

In one aspect, R₁ is alkoxy, as represented by —OR₄. In one aspect, R₁ is a saturated alkoxy. In one aspect, R₁ is an unsaturated alkoxy. In some embodiments, R₄ is C₁-C₄ alkyl, e.g., R₄ is methyl, ethyl, propyl, isopropyl, or butyl, including n-butyl, sec-butyl, isobutyl, and tert-butyl. In some embodiments, the alkoxy can be optionally substituted with one or more halo. In some aspects, R₁ is alkoxy substituted with a hydroxyl or a ketone. In one aspect, R₁ is alkoxy substituted with a halo group, i.e., a haloalkoxy, e.g., a mono-haloalkoxy, a di-haloalkoxy, or a tri-haloalkoxy. In one aspect, R₁ is a straight chain alkoxy. In one aspect, R₁ is a branched alkoxy. In one aspect, R₁ is C₁-C₄ alkoxy (—OR₄), where R₄ is C₁-C₄ alkyl, e.g., methyl or ethyl. In one aspect, R₁ is an alkoxy that includes, but is not limited to, methoxy, ethoxy, propoxy, isopropoxy, or butoxy, including for example, n-butoxy, sec-butoxy iso-butoxy, and t-butoxy.

In one aspect, R₁ is amide, which can be represented by the group “—C(═O)NR₅R₅,” in which each R₅ is independently hydro or alkyl. In some aspects, at least one R₅ of —C(═O)NR₅R₅ is hydro. In one aspect, R₅ is C₁-C₄ alkyl, e.g., methyl, ethyl, propyl, or butyl. In one aspect, R₁ is a primary amide. In one aspect, R₁ is a secondary amide. In one aspect, R₁ is a tertiary amide. In one aspect, R₁ is —C(═O)NHR₅.

In one aspect, R₁ is a short chain ester, which can be represented by the group “—C(O)OR₆” or “—CO₂R₆” where R₆ is hydro or C₁-C₄ alkyl, e.g., methyl, ethyl, propyl, or butyl.

In one aspect, R₁ is cyano.

In one aspect, X is hydro. In some embodiments, X is an alkyl. In one aspect, X is a saturated alkyl. In one aspect, X is an unsaturated alkyl. In one aspect, X is a straight chain alkyl. In one aspect, X is a branched alkyl. In one aspect, X is C₁-C₄ alkyl. In one aspect, X is methyl, ethyl, propyl, isopropyl, or butyl. In one aspect, butyl includes n-butyl, sec-butyl, isobutyl, and tert-butyl. In one aspect, X is an unsubstituted alkyl. In one aspect, X is substituted with a halo group, i.e., a haloalkyl, e.g., a mono-haloalkyl, a di-haloalkyl, or a tri-haloalkyl. In one aspect, X is a halo C₁-C₄ alkyl. In one aspect, X is a halo C₁-C₄ alkyl such as trifluoromethyl, difluoromethyl, fluoromethyl, trichloromethyl, dichloromethyl, and chloromethyl. In some embodiments, X is —OR₂, where R₂ is defined below.

In one aspect, R₂ is hydro. In some embodiments, R₂ is an alkyl. In one aspect, R₂ is a saturated alkyl. In one aspect, R₂ is an unsaturated alkyl. In one aspect, R₂ is a straight chain alkyl. In one aspect, R₂ is a branched alkyl. In one aspect, R₂ is C₁-C₄ alkyl. In one aspect, R₂ is methyl, ethyl, propyl, isopropyl, or butyl. In one aspect, R₂ is methyl. In one aspect, R₂ is ethyl. In one aspect, butyl includes n-butyl, sec-butyl, isobutyl, and tert-butyl. In one aspect, R₂ is an unsubstituted alkyl. In one aspect, R₂ is substituted with a halo group, i.e., a haloalkyl, e.g., a mono-haloalkyl, a di-haloalkyl, or a tri-haloalkyl. In one aspect, R₂ is a halo C₁-C₄ alkyl. In one aspect, R₂ is a halo C₁-C₄ alkyl such as trifluoromethyl, difluoromethyl, fluoromethyl, trichloromethyl, dichloromethyl, and chloromethyl.

In one aspect, R₃ is hydro. In some aspect, R₃ is a halo, e.g., a chloro, fluoro, or bromo. In one aspect, R₃ is alkyl. In one aspect, R₃ is a saturated alkyl. In one aspect, R₃ is an unsaturated alkyl. In one aspect, R₃ is a straight chain alkyl. In one aspect, R₃ is a branched alkyl. In one aspect, R₃ is C₁-C₄ alkyl. In one aspect, R₃ is methyl, ethyl, propyl, isopropyl, or butyl. In one aspect, butyl includes n-butyl, sec-butyl, isobutyl, and tert-butyl. In one aspect, R₃ is a haloalkyl, e.g., a mono-haloalkyl, a di-haloalkyl, or a tri-haloalkyl. In one aspect, R₃ is a C₁-C₄ alkyl optionally substituted with one or more halo. In one aspect, R₃ is a halo C₁-C₄ alkyl such as trifluoromethyl, difluoromethyl, fluoromethyl, trichloromethyl, dichloromethyl, or chloromethyl. In one aspect, R₃ is amide, which can be represented by the group “—C(O)NR₅R₅,” in which each R₅ is independently hydro or alkyl, e.g., methyl or ethyl. In one aspect, the alkyl substituent includes from 1 and up to about 8 carbon atoms. In one aspect, the alkyl substituent includes from about 1 and up to about 4 carbon atoms. In one aspect, R₃ is a primary amide. In one aspect, R₃ is a secondary amide. In one aspect, R₃ is a tertiary amide. In one aspect, R₃ is a short chain ester, which can be represented by the group “—C(O)OR₆” or “—CO₂R₆” where R₆ is hydro or C₁-C₄ alkyl, e.g., methyl or ethyl. In one aspect, R₃ is cyano. In one aspect, R₃ is alkyl substituted with one or more hydroxyl, as represented by “—(CHOH)ₙCH₂OH” wherein n is 0, 1, 2, or 3. In one aspect, R₃ is methylhydroxyl, as represented by “—CH₂OH.”

In one aspect, R₃ is alkoxy, as represented by “—OR₄.” In one aspect, R₃ is a saturated alkoxy. In one aspect, R₃ is an unsaturated alkoxy. In some embodiments, R₄ is C₁-C₄ alkyl, e.g., R₄ is methyl, ethyl, propyl, or butyl. In some embodiments, the alkoxy can be optionally substituted with one or more halo. In some aspects, R₃ is alkoxy substituted with a hydroxyl or a ketone. In one aspect, R₃ is alkoxy substituted with a halo group, i.e., a haloalkoxy, e.g., a mono-haloalkoxy, a di-haloalkoxy, or a tri-haloalkoxy. In one aspect, R₃ is a straight chain alkoxy. In one aspect, R₃ is a branched alkoxy. In one aspect, R₃ is C₁-C₄ alkoxy (—OR₄), where R₄ is C₁-C₄ alkyl, e.g., methyl or ethyl. In one aspect, R₃ is an alkoxy that includes, but is not limited to, methoxy, ethoxy, propoxy, isopropoxy, butoxy, including for example, n-butoxy, sec-butoxy iso-butoxy and t-butoxy. In one aspect, R₃ is methoxy.

In one aspect, R₇, R₈, R₉, R₁₀, or R₁, are each independently CHR₁₂, CR₁₂R₁₇ or NR₁₃. In at least one embodiment, all of R₇, R₈, R₉, R₁₀, and R₁ are CHR₁₂, where R₁₂ for each atom is independently chosen from hydro, C₁-C₄ alkyl, —OR₁₄, or —CO₂R₁₅. In at least one embodiment, all of R₇, R₈, R₉, R₁₀, and R₁ are CHR₁₂, where each R₁₂ is hydro. In at least one embodiment, all of R₇, R₈, R₉, R₁₀, and R₁₁ are CHR₁₂, where R₁₂ is each independently chosen from hydro or an unsubstituted alkyl. In one aspect, R₁₂ may be substituted with a halo group, i.e., a haloalkyl, e.g., a mono-haloalkyl, a di-haloalkyl, or a tri-haloalkyl. In one aspect, R₁₂ may be a halo C₁-C₄ alkyl, for instance trifluoromethyl, difluoromethyl, fluoromethyl, trichloromethyl, dichloromethyl, and chloromethyl. In at least one embodiment, all of R₇, R₈, R₉, R₁₀, and R₁₁ are CHR₁₂, where each R₁₂ is independently chosen from hydro, C₁-C₄ alkyl, or —OR₁₄, where R₁₄ is further chosen from hydro or C₁-C₄ alkyl. In some embodiments, R₁₂ is alkoxy, such as methoxy, ethoxy, propoxy, isopropoxy, butoxy, including for example, n-butoxy, sec-butoxy iso-butoxy and t-butoxy. In at least one embodiment, all of R₇, R₈, R₉, R₁₀, and R₁₁ are CHR₁₂, where each R₁₂ is independently chosen from hydro, C₁-C₄ alkyl, —OR₁₄, or —CO₂R₁₅, where each of R₁₄ and R₁₅ is hydro or C₁-C₄ alkyl, e.g., methyl, ethyl, propyl, or butyl.

In one aspect, at least one of R₇, R₈, R₉, R₁₀, and R₁₁ is CR₁₂R₁₇, where R₁₂ and R₁₇ are each independently chosen from hydro or C₁-C₄ alkyl, e.g., methyl, ethyl, propyl, or butyl. In another embodiment, at least two of R₇, R₈, R₉, R₁₀, and R₁₁ are CR₁₂R₁₇, where R₁₂ and R₁₇ are each independently chosen from hydro or C₁-C₄ alkyl, e.g., methyl, ethyl, propyl, or butyl. In at least one aspect, R₇ is CR₁₂R₁₇, where R₁₂ is methyl and R₁₇ is methyl, and R₈, R₉, R₁₀, and R₁₁ are each independently chosen from CHR₁₂ or NR₁₃. In at least one aspect, R₈ is CR₁₂R₁₇, where R₁₂ is methyl and R₁₇ is methyl, and R₇, R₉, R₁₀, and R₁₁ are each independently chosen from CHR₁₂ or NR₁₃. In at least one aspect, R₉ is CR₁₂R₁₇, where R₁₂ is methyl and R₁₇ is methyl, and R₇, R₈, R₁₀, and R₁₁ are each independently chosen from CHR₁₂ or NR₁₃. In at least one aspect, R₁₀ is CR₁₂R₁₇, where R₁₂ is methyl and R₁₇ is methyl, and R₇, R₈, R₉, and R₁₁ are each independently chosen from CHR₁₂ or NR₁₃. In at least one aspect, R₁₁ is CR₁₂R₁₇, where R₁₂ is methyl and R₁₇ is methyl, and R₇, R₈, R₉, and R₁₀ are each independently chosen from CHR₁₂ or NR₁₃.

In one aspect, at least one of R₇, R₈, R₉, R₁₀, and R₁₁ is NR₁₃, where R₁₃ is hydro or C₁-C₄ alkyl, e.g., methyl, ethyl, propyl, or butyl. In another embodiment, at least two of R₇, R₈, R₉, R₁₀, and R₁₁ are NR₁₃, where each of the two R₁₃ groups are independently hydro or C₁-C₄ alkyl, e.g., methyl, ethyl, propyl, or butyl. In one aspect, R₇ is NR₁₃ and R₈, R₉, R₁₀, and Ru are CHR₁₂, where R₁₃ is hydro or C₁-C₄ alkyl, e.g., methyl, ethyl, propyl, or butyl and R₁₂ is hydro. In one aspect, R₈ is NR₁₃ and R₇, R₉, R₁₀, and R₁₁ are CHR₁₂, where R₁₃ is hydro or C₁-C₄ alkyl, e.g., methyl, ethyl, propyl, or butyl and R₁₂ is hydro. In one aspect, R₉ is NR₁₃ and R₇, R₈, R₁₀, and R₁₁ are CHR₁₂, where R₁₃ is hydro or C₁-C₄ alkyl, e.g., methyl, ethyl, propyl, or butyl and R₁₂ is hydro. In one aspect, R₁₀ is NR₁₃ and R₇, R₈, R₉, and Ru are CHR₁₂ where R₁₃ is hydro or C₁-C₄ alkyl, e.g., methyl, ethyl, propyl, or butyl and R₁₂ is hydro. In one aspect, Ru is NR₁₃ and R₇, R₈, R₉, and R₁₀ are CHR₁₂ where R₁₃ is hydro or C₁-C₄ alkyl, e.g., methyl, ethyl, propyl, or butyl and R₁₂ is hydro.

In one aspect, R₇ is NR₁₃ and R₈, R₉, R₁₀, and R₁₁ are CHR₁₂, where R₁₃ is hydro, C₁-C₄ alkyl, —(CH₂)_{y}OH, —OR₁₄, —CO₂R₁₅, or —C(═O)R₁₆, and R₁₂ is hydro. In one aspect, R₈ is NR₁₃ and R₇, R₉, R₁₀, and R₁₁ are CHR₁₂, where R₁₃ is hydro, C₁-C₄ alkyl, —(CH₂)_{y}OH, —OR₁₄, —CO₂R₁₅, or —C(═O)R₁₆, and R₁₂ is hydro. In one aspect, R₉ is NR₁₃ and R₇, R₈, R₁₀, and R₁₁ are CHR₁₂, where R₁₃ is hydro, C₁-C₄ alkyl, —(CH₂)_{y}OH, —OR₁₄, —CO₂R₁₅, or —C(═O)R₁₆, and R₁₂ is hydro. In one aspect, R₁₀ is NR₁₃ and R₇, R₈, R₉, and Ru are CHR₁₂, where R₁₃ is hydro, C₁-C₄ alkyl, —(CH₂)_{y}OH, —OR₁₄, —CO₂R₁₅, or —C(═O)R₁₆, and R₁₂ is hydro. In one aspect, Ru is NR₁₃ and R₇, R₈, R₉, and R₁₀ are CHR₁₂, where R₁₃ is hydro, C₁-C₄ alkyl, —(CH₂)_{y}OH, —OR₁₄, —CO₂R₁₅, or —C(═O)R₁₆, and R₁₂ is hydro. In any of these instances, y is chosen from 0, 1, 2, or 3, and R₁₄, R₁₅, and R₁₆ are each independently chosen from hydro and C₁-C₄ alkyl.

In one aspect, R₉ is NR₁₃ and R₇, R₈, R₁₀, and R₁₁ are CHR₁₂, where each R₁₂ and R₁₃ are hydro. In one aspect, R₉ is NR₁₃ and R₇, R₈, R₁₀, and R₁₁ are CHR₁₂, where each R₁₂ is hydro, and R₁₃ is C₁-C₄ alkyl. In one aspect, R₉ is NR₁₃ and R₇, R₈, R₁₀, and R₁₁ are CHR₁₂, where each R₁₂ is hydro, and R₁₃ is methyl.

In one aspect, R₉ is NR₁₃, where R₁₃ is hydro; at least one of R₇, R₈, R₁₀, and R₁₁ is CR₁₂R₁₇; where R₁₂ and R₁₇ are each C₁-C₄ alkyl; and the remaining of R₇, R₈, R₁₀, and R₁₁ are CHR₁₂, where each R₁₂ is hydro, and R₁₃ is hydro or C₁-C₄ alkyl. In one aspect, R₉ is NR₁₃, where R₁₃ is hydro; R₇, R₁₀, and R₁₁ are CHR₁₂, where R₁₂ is hydro; and R₈ is CR₁₂R₁₇, where each of R₁₂ and R₁₇ is methyl. In one aspect, R₉ is NR₁₃, where R₁₃ is hydro; R₇, R₈, and R₁₁ are CHR₁₂; where R₁₂ is hydro; and R₁₀ is CR₁₂R₁₇, where each of R₁₂ and R₁₇ is methyl. In one aspect, R₉ is NR₁₃, where R₁₃ is hydro; R₇ and R₁₁ are CHR₁₂, where R₁₂ is hydro; and R₈ and R₁₀ are CR₁₂R₁₇, where each of R₁₂ and R₁₇ is methyl.

In another aspect, the compound has a structure represented by Formula Ia:

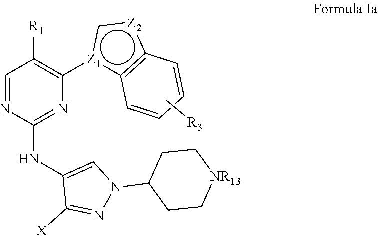

wherein
one of Z₁ and Z₂ is N, and the other is C;
R₁ is hydro, halo, C₁-C₄ alkyl, —OR₄, —C(═O)NR₅R₅, —CO₂R₆, or cyano;
X is hydro, C₁-C₄ alkyl, or —OR₂;
R₂ is hydro or C₁-C₄ alkyl;
R₃ is hydro, halo, C₁-C₄ alkyl, —(CH₂)_{y}OH, —OR₄, —C(═O)NR₅R₅, —CO₂R₆, or cyano;
R₄ is C₁-C₄ alkyl;
R₅ is hydro or C₁-C₄ alkyl, e.g., methyl or ethyl;
R₆ is hydro or C₁-C₄ alkyl, e.g., methyl or ethyl;
R₁₃ is hydro, C₁-C₄ alkyl, —(CH₂)_{y}OH, —OR₁₄, —CO₂R₁₅, or —C(═O)R₁₆;
R₁₆ is hydro or C₁-C₄ alkyl;
y is 0, 1, 2, or 3; and
wherein one or more of the alkyl are optionally substituted with one or more halo, or a pharmaceutically acceptable salt thereof.

In another aspect, the compound has a structure represented by Formula Ib or Formula Ic:

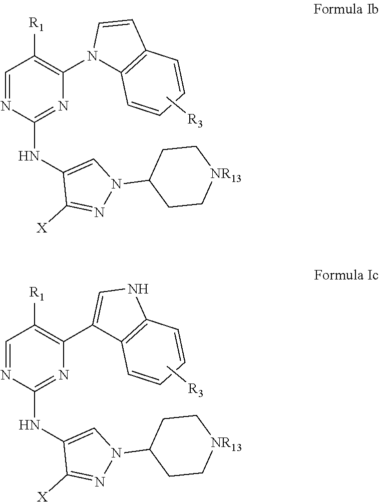

wherein
R₁ is hydro, halo, C₁-C₄ alkyl, —OR₄, —C(═O)NR₅R₅, —CO₂R₆, or cyano;
X is hydro, C₁-C₄ alkyl, or —OR₂;
R₂ is hydro or C₁-C₄ alkyl;
R₃ is hydro, halo, C₁-C₄ alkyl, —(CH₂)_{y}OH, —OR₄, —C(═O)NR₅R₅, —CO₂R₆, or cyano;
R₄ is C₁-C₄ alkyl;
R₅ is hydro or C₁-C₄ alkyl, e.g., methyl or ethyl;
R₆ is hydro or C₁-C₄ alkyl, e.g., methyl or ethyl;
R₁₃ is hydro, C₁-C₄ alkyl, —(CH₂)_{y}OH, —OR₁₄, —CO₂R₁₅, or —C(═O)R₁₆;
R₁₆ is hydro or C₁-C₄ alkyl;
y is 0, 1, 2, or 3; and
wherein one or more of the alkyl are optionally substituted with one or more halo, or a pharmaceutically acceptable salt thereof.

In another aspect, a compound of Formula Ia, Formula Ib, or Formula Ic is provided, wherein
R₁ is hydro, halo, C₁-C₄ alkyl, —OR₄, —C(═O)NR₅R₅, —CO₂R₆, or cyano;
X is —OR₂;
R₂ is hydro or C₁-C₄ alkyl;
R₃ is hydro, halo, C₁-C₄ alkyl, —OR₄, —C(═O)NR₅R₅, —CO₂R₆, or cyano;
R₄ is C₁-C₄ alkyl;
R₅ is hydro, methyl or ethyl;
R₆ is hydro, methyl or ethyl; and
R₁₃ is hydro, wherein one or more of the alkyl are optionally substituted with one or more halo, or a pharmaceutically acceptable salt thereof.

In one aspect, R₁ is hydro. In one aspect, R₁ is halo, including, for example, chloro, fluoro, bromo, or iodo. In one aspect, R₁ is bromo, chloro, or fluoro. In one aspect, R₁ is fluoro. In one aspect, R₁ is alkyl. In one aspect, R₁ is a saturated alkyl. In one aspect, R₁ is an unsaturated alkyl, e.g., an alkenyl or alkynyl. In some aspects, R₁ can be polyunsaturated. In one aspect, R₁ is a straight chain alkyl. In one aspect, R₁ is a branched alkyl. In one aspect, R₁ is C₁-C₄ alkyl. In one aspect, R₁ is methyl, ethyl, propyl, isopropyl, or butyl. In one aspect, butyl includes n-butyl, sec-butyl, isobutyl, and tert-butyl. In one aspect, R₁ is a substituted alkyl, e.g., substituted with one or more halo. In some embodiments, R₁ is a C₁-C₄ alkyl, wherein at least one carbon of the alkyl is substituted with one or more chloro or fluoro. In some aspects, R₁ is substituted with a hydroxyl or a ketone. In one aspect, R₁ is an unsubstituted alkyl. In one aspect, R₁ is substituted with a halo group, i.e., a haloalkyl, e.g., a mono-haloalkyl, a di-haloalkyl, or a tri-haloalkyl. In one aspect, R₁ is a halo C₁-C₄ alkyl. In one aspect, R₁ is a halo C₁-C₄ alkyl such as trifluoromethyl, difluoromethyl, fluoromethyl, trichloromethyl, dichloromethyl, and chloromethyl.

In one aspect, R₁ is alkoxy, as represented by “—OR₄”. In one aspect, R₁ is a saturated alkoxy. In one aspect, R₁ is an unsaturated alkoxy. In some embodiments, R₄ is C₁-C₄ alkyl, e.g., R₄ is methyl, ethyl, propyl, isopropyl, or butyl, including n-butyl, sec-butyl, isobutyl, and tert-butyl. In some embodiments, the alkoxy can be optionally substituted with one or more halo. In some aspects, R₁ is alkoxy substituted with a hydroxyl, or a ketone. In one aspect, R₁ is alkoxy substituted with a halo group, i.e., a haloalkoxy, e.g., a mono-haloalkoxy, a di-haloalkoxy, or a tri-haloalkoxy. In one aspect, R₁ is a straight chain alkoxy. In one aspect, R₁ is a branched alkoxy. In one aspect, R₁ is C₁-C₄ alkoxy (—OR₄), where R₄ is C₁-C₄ alkyl, e.g., methyl or ethyl. In one aspect, R₁ is an alkoxy that includes, but is not limited to, methoxy, ethoxy, propoxy, isopropoxy, or butoxy, including for example, n-butoxy, sec-butoxy iso-butoxy and t-butoxy.

In one aspect, R₁ is amide, which can be represented by the group “—C(═O)NR₅R₅,” in which each R₅ is independently hydro or alkyl. In some aspects, at least one R₅ of “—C(═O)NR₅R₅” is hydro. In one aspect, R₅ is C₁-C₄ alkyl, e.g., methyl, ethyl, propyl, or butyl. In one aspect, R₁ is a primary amide. In one aspect, R₁ is a secondary amide. In one aspect, R₁ is a tertiary amide. In one aspect, R₁ is —C(═O)NHR₅.

In one aspect R₁ is a short chain ester, which can be represented by the group “—C(O)OR₆” or “—CO₂R₆” where R₆ is hydro or C₁-C₄ alkyl, e.g., methyl or ethyl.

In one aspect, R₁ is cyano.

In one aspect, X is hydro. In one aspect, X is C₁-C₄ alkyl, e.g., methyl, ethyl, propyl, or butyl. In one aspect, X is —OR₂.

In one aspect, R₂ is hydro. In some embodiments, R₂ is an alkyl. In one aspect, R₂ is a saturated alkyl. In one aspect, R₂ is an unsaturated alkyl. In one aspect, R₂ is a straight chain alkyl. In one aspect, R₂ is a branched alkyl. In one aspect, R₂ is C₁-C₄ alkyl. In one aspect, R₂ is methyl, ethyl, propyl, isopropyl, or butyl. In one aspect, R₂ is methyl. In one aspect, R₂ is ethyl. In one aspect, butyl includes n-butyl, sec-butyl, isobutyl, and tert-butyl.

In one aspect, R₃ is hydro. In some aspects, R₃ is a halo, e.g., a chloro, fluoro or bromo. In one aspect, R₃ is alkyl. In one aspect, R₃ is a saturated alkyl. In one aspect, R₃ is an unsaturated alkyl. In one aspect, R₃ is a straight chain alkyl. In one aspect, R₃ is a branched alkyl. In one aspect, R₃ is C₁-C₄ alkyl. In one aspect, R₃ is methyl, ethyl, propyl, isopropyl, or butyl. In one aspect, butyl includes n-butyl, sec-butyl, isobutyl, and tert-butyl. In one aspect, R₃ is a haloalkyl, e.g., a mono-haloalkyl, a di-haloalkyl, or a tri-haloalkyl. In one aspect, R₃ is a C₁-C₄ alkyl optionally substituted with one or more halo. In one aspect, R₃ is a halo C₁-C₄ alkyl such as trifluoromethyl, difluoromethyl, fluoromethyl, trichloromethyl, dichloromethyl, or chloromethyl. In one aspect, R₃ is amide, which can be represented by the group “—C(O)NR₅R₅,” in which each R₅ is independently hydro or alkyl, e.g., methyl or ethyl. In one aspect, the alkyl substituent includes from 1 and up to about 8 carbon atoms. In one aspect, the alkyl substituent includes from about 1 and up to about 4 carbon atoms. In one aspect, R₃ is a primary amide. In one aspect, R₃ is a secondary amide. In one aspect, R₃ is a tertiary amide. In one aspect, R₃ is a short chain ester, which can be represented by the group “—C(O)OR₆” or “—CO₂R₆” where R₆ is hydro or C₁-C₄ alkyl, e.g., methyl or ethyl. In one aspect, R₃ is cyano. In one aspect, R₃ is alkyl substituted with one or more hydroxyl, as represented by “—(CHOH)ₙCH₂OH” wherein n is 0, 1, 2, or 3. In one aspect, R₃ is methylhydroxyl, as represented by “—CH₂OH.”

In one aspect, the compounds of Formula Ia, Formula Ib, and Formula Ic as described herein can include, by way of non-limiting example, any of the following as found in the Table 2A below:

TABLE 2A
Compounds of Formula Ia, Ib, and Ic,
where X is OR₂ and R₁₃ is H or methyl

| R₁ | R₂ | R₃ |
|---|---|---|
| chloro | methyl | cyano |
| chloro | ethyl | cyano |
| fluoro | methyl | cyano |
| fluoro | ethyl | cyano |
| fluoromethyl | methyl | cyano |
| fluoromethyl | ethyl | cyano |
| difluoromethyl | methyl | cyano |
| difluoromethyl | ethyl | cyano |
| trifluoromethyl | methyl | cyano |
| trifluoromethyl | ethyl | cyano |
| chloromethyl | methyl | cyano |
| chloromethyl | ethyl | cyano |
| dichloromethyl | methyl | cyano |
| dichloromethyl | ethyl | cyano |
| trichloromethyl | methyl | cyano |
| trichloromethyl | ethyl | cyano |
| methyl | methyl | cyano |
| methyl | ethyl | cyano |
| ethyl | methyl | cyano |
| ethyl | ethyl | cyano |
| chloro | methyl | hydro |
| chloro | ethyl | hydro |
| fluoro | methyl | hydro |
| fluoro | ethyl | hydro |
| fluoromethyl | methyl | hydro |
| fluoromethyl | ethyl | hydro |
| difluoromethyl | methyl | hydro |
| difluoromethyl | ethyl | hydro |
| trifluoromethyl | methyl | hydro |
| trifluoromethyl | ethyl | hydro |
| chloromethyl | methyl | hydro |
| chloromethyl | ethyl | hydro |
| dichloromethyl | methyl | hydro |
| dichloromethyl | ethyl | hydro |
| trichloromethyl | methyl | hydro |
| trichloromethyl | ethyl | hydro |
| methyl | methyl | hydro |
| methyl | ethyl | hydro |
| ethyl | methyl | hydro |
| ethyl | ethyl | hydro |
| chloro | methyl | methoxy |
| chloro | ethyl | methoxy |
| fluoro | methyl | methoxy |
| fluoro | ethyl | methoxy |
| fluoromethyl | methyl | methoxy |
| fluoromethyl | ethyl | methoxy |
| difluoromethyl | methyl | methoxy |
| difluoromethyl | ethyl | methoxy |
| trifluoromethyl | methyl | methoxy |
| trifluoromethyl | ethyl | methoxy |
| chloromethyl | methyl | methoxy |
| chloromethyl | ethyl | methoxy |
| dichloromethyl | methyl | methoxy |
| dichloromethyl | ethyl | methoxy |
| trichloromethyl | methyl | methoxy |
| trichloromethyl | ethyl | methoxy |
| methyl | methyl | methoxy |
| methyl | ethyl | methoxy |
| ethyl | methyl | methoxy |
| ethyl | ethyl | methoxy |

In one aspect, the compound has a structure represented by Formula Ia, Ib, or Ic, and wherein R₁, X, R₃, and R₁₃ are as found in Table 2B.

TABLE 2B

| R₁ | X | R₃ | R₁₃ |
|---|---|---|---|
| chloro | methoxy | cyano | hydro |
| | methoxy | methoxy | hydro |
| | ethoxy | hydro | hydro |
| | methoxy | cyano | methyl |
| | methoxy | hydro | hydro |
| | isopropoxy | hydro | hydro |
| | methoxy | ethylhydroxy | hydro |
| fluoro | methoxy | cyano | hydro |
| | methoxy | cyano | methyl |
| | methoxy | cyano | isopropyl |
| | methoxy | cyano | n-propyl |
| | methoxy | cyano | ethyl |
| | methoxy | cyano | n-propylhydroxy |
| | ethoxy | cyano | methyl |
| | methoxy | cyano | methyl |
| | methoxy | cyano | carboxymethyl |
| | hydro | cyano | hydro |
| | ethoxy | cyano | hydro |
| | methoxy | cyano | hydro |
| | methoxy | hydro | hydro |
| | methoxy | methyl | hydro |
| | ethoxy | hydro | hydro |
| trifluoromethyl | methoxy | cyano | hydro |
| | ethoxy | hydro | hydro |
| | ethoxy | hydro | methyl |
| | methoxy | cyano | methyl |
| | methoxy | hydro | hydro |
| methyl | methoxy | hydro | hydro |
| cyano | methoxy | trifluoromethyl | hydro |
| bromo | methoxy | hydro | hydro |

In one aspect, the compound has a structure represented by Formula I, wherein R₁ is halo, X is —OR₂, R₂ is methyl, R₃ is cyano, R₇, R₈, R₁₀, and R₁ are CHR₁₂, where R₁₂ is hydro, and R₉ is NR₁₃, where R₁₃ is C₁-C₄ alkyl or —(CH₂)_{y}OH, where y is 1-3.

In one aspect, the compound has a structure represented by Formula I wherein R₁ is fluoro, X is —OR₂, R₂ is methyl, R₃ is cyano, R₇, R₈, R₁₀, and R₁ are CHR₁₂, where R₁₂ is hydro, and R₉ is NR₁₃, where R₁₃ is C₁-C₄ alkyl.

In one aspect, the compound has a structure represented by Formula I wherein R₁ is fluoro, X is —OR₂, R₂ is methyl, R₃ is cyano, R₇, R₈, R₁₀, and R₁ are CHR₁₂, where R₁₂ is hydro, and R₉ is NR₁₃, where R₁₃ is methyl.

In one aspect, the compound has a structure represented by Formula I wherein Z₁ is N, Z₂ is C, R₁ is fluoro, X is —OR₂, R₂ is methyl, R₃ is cyano, R₇, R₈, R₁₀, and R₁₁ are CHR₁₂, where R₁₂ is hydro, and R₉ is NR₁₃, where R₁₃ is hydro.

In one aspect, the compound has a structure represented by Formula I wherein Z₁ is N, Z₂ is C, R₁ is fluoro, X is —OR₂, R₂ is ethyl, R₃ is cyano, R₇, R₈, R₁₀, and R₁₁ are CHR₁₂, where R₁₂ is hydro, and R₉ is NR₁₃, where R₁₃ is methyl.

In one aspect, the compound has a structure represented by Formula I wherein Z₁ is N, Z₂ is C, R₁ is fluoro, X is —OR₂, R₂ is methyl, R₃ is cyano, R₇, R₁₀, and R₁₁ are CHR₁₂, where R₁₂ is hydro, R₈ is CR₁₂R₁₇, where R₁₂ and R₁₇ are each methyl, and R₉ is NR₁₃, where R₁₃ is methyl.

In one aspect, the compound has a structure represented by Formula I wherein R₁ is fluoro, X is —OR₂, R₂ is methyl, R₃ is cyano, R₇, R₈, R₁₀, and R₁₁ are CHR₁₂, where R₁₂ is hydro, and R₉ is NR₁₃, where R₁₃ is (CH₂)_{y}OH, where y is 1.

In one aspect, the compound has a structure represented by Formula I wherein R₁ is fluoro, X is —OR₂, R₂ is methyl, R₃ is cyano, R₇, R₈, R₁₀, and R₁₁ are CHR₁₂, where R₁₂ is hydro, and R₉ is NR₁₃, where R₁₃ is (CH₂)_{y}OH, where y is 2.

In one aspect, the compound has a structure represented by Formula I wherein R₁ is fluoro, X is —OR₂, R₂ is methyl, R₃ is cyano, R₇, R₈, R₁₀, and R₁ are CHR₁₂, where R₁₂ is hydro, and R₉ is NR₁₃, where R₁₃ is (CH₂)_{y}OH, where y is 3.

In one aspect, the compound has a structure represented by Formula Ia, Formula Ib, or Formula Ic, wherein R₁ is halo, X is —OR₂, R₂ is ethyl or methyl, R₃ is cyano, and R₁₃ is hydro, C₁-C₄ alkyl, —C(═O)R₁₆, or —(CH₂)_{y}OH. In some embodiments, R₁ is fluoro, R₂ is ethyl, and R₁₃ is hydro. In some embodiments, R₁ is fluoro, R₂ is ethyl, and R₁₃ is methyl, ethyl, n-propyl, or isopropyl. In some embodiments, R₁ is fluoro, R₂ is ethyl, and R₁₃ is —C(═O)R₁₆, e.g., —C(═O)CH₃. In some embodiments, R₁ is fluoro, R₂ is ethyl, and R₁₃ is —(CH₂)_{y}OH, e.g., —(CH₂)₃OH. In some embodiments, R₁ is fluoro, R₂ is methyl, and R₁₃ is hydro. In some embodiments, R₁ is fluoro, R₂ is methyl, and R₁₃ is methyl, ethyl, n-propyl, or isopropyl. In some embodiments, R₁ is fluoro, R₂ is methyl, and R₁₃ is —C(═O)R₁₆, e.g., —C(═O)CH₃. In some embodiments, R₁ is fluoro, R₂ is methyl, and R₁₃ is —(CH₂)_{y}OH, e.g., —(CH₂)₃OH. In some embodiments, R₁ is chloro, R₂ is ethyl, and R₁₃ is methyl, ethyl, n-propyl, or isopropyl. In some embodiments, R₁ is chloro, R₂ is ethyl, and R₁₃ is —C(═O)R₁₆, e.g., —C(═O)CH₃. In some embodiments, R₁ is chloro, R₂ is ethyl, and R₁₃ is —(CH₂)_{y}OH, e.g., —(CH₂)₃OH. In some embodiments, R₁ is chloro, R₂ is methyl, and R₁₃ is hydro. In some embodiments, R₁ is chloro, R₂ is methyl, and R₁₃ is methyl, ethyl, n-propyl, or isopropyl. In some embodiments, R₁ is chloro, R₂ is methyl, and R₁₃ is —C(═O)R₁₆, e.g., —C(═O)CH₃. In some embodiments, R₁ is chloro, R₂ is methyl, and R₁₃ is —(CH₂)_{y}OH, e.g., —(CH₂)₃OH.

In one aspect, the compound has a structure represented by Formula Ia, Formula Ib, or Formula Ic, wherein R₁ is haloalkyl, X is —OR₂, R₂ is ethyl or methyl, R₃ is cyano, and R₁₃ is hydro or methyl. In some embodiments, R₁ is haloalkyl, e.g., trifluoromethyl, R₂ is ethyl, R₃ is cyano, and R₁₃ is hydro. In some embodiments, R₁ is haloalkyl, e.g., trifluoromethyl, R₂ is ethyl, R₃ is cyano, and R₁₃ is methyl. In some embodiments, R₁ is haloalkyl, e.g., trifluoromethyl, R₂ is methyl, R₃ is cyano, and R₁₃ is hydro. In some embodiments, R₁ is haloalkyl, e.g., trifluoromethyl, R₂ is methyl, R₃ is cyano, and R₁₃ is methyl.

In one aspect, the compound has a structure represented by Formula Ia, Formula Ib, or Formula Ic, wherein R₁ is halo, X is —OR₂, R₂ is ethyl or methyl, R₃ is methyl or methoxy, and R₁₃ is hydro. In some embodiments, R₁ is fluoro, R₂ is ethyl, R₃ is methyl, and R₁₃ is hydro. In some embodiments, R₁ is fluoro, R₂ is methyl, R₃ is methoxy, and R₁₃ is hydro. In some embodiments, R₁ is fluoro, R₂ is methyl, R₃ is methyl, and R₁₃ is hydro. In some embodiments, R₁ is fluoro, R₂ is ethyl, R₃ is methoxy, and R₁₃ is hydro. In some embodiments, R₁ is chloro, R₂ is ethyl, R₃ is methyl, and R₁₃ is hydro. In some embodiments, R₁ is chloro, R₂ is methyl, R₃ is methoxy, and R₁₃ is hydro. In some embodiments, R₁ is chloro, R₂ is methyl, R₃ is methyl, and R₁₃ is hydro. In some embodiments, R₁ is chloro, R₂ is ethyl, R₃ is methoxy, and R₁₃ is hydro.

In one aspect, the compound has a structure represented by Formula Ia, Formula Ib, or Formula Ic, wherein R₁ is halo, X is —OR₂, R₂ is C₁-C₄ alkyl, R₃ is hydro, and R₁₃ is hydro. In some embodiments, R₁ is fluoro, R₂ is methyl, R₃ is hydro, and R₁₃ is hydro. In some embodiments, R₁ is chloro, R₂ is methyl, R₃ is hydro, and R₁₃ is hydro. In some embodiments, R₁ is bromo, R₂ is methyl, R₃ is hydro, and R₁₃ is hydro. In some embodiments, R₁ is fluoro, R₂ is ethyl, R₃ is hydro, and R₁₃ is hydro. In some embodiments, R₁ is chloro, R₂ is ethyl, R₃ is hydro, and R₁₃ is hydro. In some embodiments, R₁ is bromo, R₂ is ethyl, R₃ is hydro, and R₁₃ is hydro. In some embodiments, R₁ is fluoro, R₂ is propyl, e.g., isopropyl, R₃ is hydro, and R₁₃ is hydro. In some embodiments, R₁ is chloro, R₂ is propyl, e.g., isopropyl, R₃ is hydro, and R₁₃ is hydro. In some embodiments, R₁ is bromo, R₂ is propyl, e.g., isopropyl, R₃ is hydro, and R₁₃ is hydro.

In one aspect, the compound has a structure represented by Formula Ia, Formula Ib, or Formula Ic, wherein R₁ is haloalkyl, X is —OR₂, R₂ is ethyl or methyl, R₃ is hydro, and R₁₃ is hydro or methyl. In some embodiments, R₁ is trifluoromethyl, R₂ is methyl, R₃ is hydro, and R₁₃ is hydro. In some embodiments, R₁ is trifluoromethyl, R₂ is methyl, R₃ is hydro, and R₁₃ is hydro. In some embodiments, R₁ is trifluoromethyl, R₂ is ethyl, R₃ is hydro, and R₁₃ is hydro. In some embodiments, R₁ is trifluoromethyl, R₂ is ethyl, R₃ is hydro, and R₁₃ is hydro. In some embodiments, R₁ is trifluoromethyl, R₂ is methyl, R₃ is hydro, and R₁₃ is methyl. In some embodiments, R₁ is trifluoromethyl, R₂ is methyl, R₃ is hydro, and R₁₃ is methyl. In some embodiments, R₁ is trifluoromethyl, R₂ is ethyl, R₃ is hydro, and R₁₃ is methyl. In some embodiments, R₁ is trifluoromethyl, R₂ is ethyl, R₃ is hydro, and R₁₃ is methyl.

In one aspect, the compound has a structure represented by Formula Ia, Formula Ib, or Formula Ic, wherein R₁ is halo, X is hydro, R₃ is cyano, and R₁₃ is hydro. In some embodiments, R₁ is fluoro. In some embodiments, R₁ is chloro. In some embodiments, R₁ is bromo.

In one aspect, the compound has a structure represented by Formula Ia, Formula Ib, or Formula Ic, wherein R₁ is cyano, X is —OR₂, R₂ is ethyl or methyl, R₃ is haloalkyl, and R₁₃ is hydro. In some embodiments, R₁ is cyano, R₂ is methyl, R₃ is trifluoromethyl, and R₁₃ is hydro. In some embodiments, R₁ is cyano, R₂ is ethyl, R₃ is trifluoromethyl, and R₁₃ is hydro.

In one aspect, the compound has a structure represented by Formula Ia, Formula Ib, or Formula Ic, wherein R₁ is ethyl or methyl, X is —OR₂, R₂ is ethyl or methyl, R₃ is hydro, and R₁₃ is hydro. In some embodiments, R₁ is methyl, R₂ is methyl, R₃ is hydro, and R₁₃ is hydro. In some embodiments, R₁ is methyl, R₂ is ethyl, R₃ is hydro, and R₁₃ is hydro. In some embodiments, R₁ is ethyl, R₂ is methyl, R₃ is hydro, and R₁₃ is hydro. In some embodiments, R₁ is ethyl, R₂ is ethyl, R₃ is hydro, and R₁₃ is hydro.

In one aspect, the compound has a structure represented by Formula Ia, Formula Ib, or Formula Ic, wherein R₁ is halo, X is —OR₂, R₂ is ethyl or methyl, R₃ is —(CH₂)_{y}OH, and R₁₃ is hydro, wherein y is 0, 1, 2, or 3. In some embodiments, R₁ is fluoro, R₂ is methyl, R₃ is —OH, and R₁₃ is hydro. In some embodiments, R₁ is fluoro, R₂ is methyl, R₃ is —CH₂OH, and R₁₃ is hydro. In some embodiments, R₁ is fluoro, R₂ is methyl, R₃ is —(CH₂)₂OH, and R₁₃ is hydro. In some embodiments, R₁ is fluoro, R₂ is methyl, R₃ is —(CH₂)₃OH, and R₁₃ is hydro. In some embodiments, R₁ is chloro, R₂ is methyl, R₃ is —OH, and R₁₃ is hydro. In some embodiments, R₁ is chloro, R₂ is methyl, R₃ is —CH₂OH, and R₁₃ is hydro. In some embodiments, R₁ is chloro, R₂ is methyl, R₃ is —(CH₂)₂OH, and R₁₃ is hydro. In some embodiments, R₁ is chloro, R₂ is methyl, R₃ is —(CH₂)₃OH, and R₁₃ is hydro. In some embodiments, R₁ is fluoro, R₂ is ethyl, R₃ is —OH, and R₁₃ is hydro. In some embodiments, R₁ is fluoro, R₂ is ethyl, R₃ is —CH₂OH, and R₁₃ is hydro. In some embodiments, R₁ is fluoro, R₂ is ethyl, R₃ is —(CH₂)₂OH, and R₁₃ is hydro. In some embodiments, R₁ is fluoro, R₂ is ethyl, R₃ is —(CH₂)₃OH, and R₁₃ is hydro. In some embodiments, R₁ is chloro, R₂ is ethyl, R₃ is —OH, and R₁₃ is hydro. In some embodiments, R₁ is chloro, R₂ is ethyl, R₃ is —CH₂OH, and R₁₃ is hydro. In some embodiments, R₁ is chloro, R₂ is ethyl, R₃ is —(CH₂)₂OH, and R₁₃ is hydro. In some embodiments, R₁ is chloro, R₂ is ethyl, R₃ is —(CH₂)₃OH, and R₁₃ is hydro.

In one aspect, the compound has a structure represented by Formula Ia, Ib, or Ic, wherein R₁ is fluoro, X is —OR₂, R₂ is methyl, R₃ is cyano, and R₁₃ is hydro.

In one aspect, the compound has a structure represented by Formula Ia, wherein R₁ is fluoro, X is —OR₂, R₂ is methyl, R₃ is cyano, and R₁₃ is hydro.

In one aspect, the compound has a structure represented by Formula Ia, wherein R₁ is fluoro, X is —OR₂, R₂ is ethyl, R₃ is cyano, and R₁₃ is methyl.

In one aspect, the compound has a structure represented by Formula Ib, wherein R₁ is fluoro, X is —OR₂, R₂ is methyl, R₃ is cyano, and R₁₃ is hydro.

In one aspect, the compound has a structure represented by Formula Ib, wherein R₁ is fluoro, X is —OR₂, R₂ is ethyl, R₃ is cyano, and R₁₃ is methyl.

In some embodiments, the compound comprises a structure as shown in Table 1. In some embodiments, the compound is any one of Compounds 2-7 as shown in Table 1. In some embodiments, the compound is Compound 4 as shown in Table 1. In some embodiments, the compound is Compound 8 as shown in Table 1. In some embodiments, the compound is Compound 32 as shown in Table 1. In some embodiments, the compound is any one of Compounds 32a or 32b as shown below.

In one aspect, the compounds represented by Formula I, Ia, Ib, or Ic may have one or more stereocenters and may exist as racemates or racemic mixtures, single enantiomers, individual diastereomers and diastereomeric mixtures. Stereoisomers may be separated using conventional techniques, e.g., chromatography or fractional crystallization, or the stereoisomers may be made by stereoselective synthesis. For instance, Compounds 32-35 contain at least one stereocenter and their individual isomers (further depicted below) are all disclosed herein.

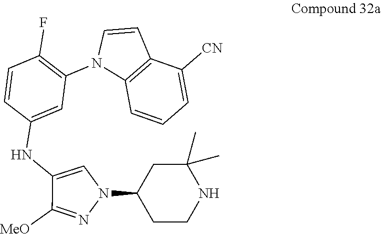 [SMILES: COc1nn([C@@H]2CCNC(C)(C)C2)cc1Nc1ccc(F)c(-n2ccc3c(C#N)cccc32)c1]

### (S)-1-(2-((1-(2,2-dimethylpiperidin-4-yl)-3-methoxy-1H-pyrazol-4-yl)amino)-5-fluoropyrimidin-4-yl)-1H-indole-4-carbonitrile

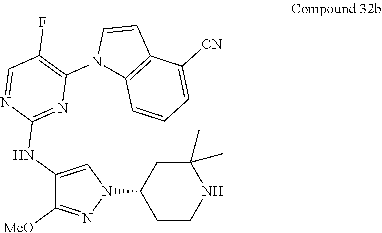 [SMILES: COc1nn([C@H]2CCNC(C)(C)C2)cc1Nc1ncc(F)c(-n2ccc3c(C#N)cccc32)n1]

### (R)-1-(2-((1-(2,2-dimethylpiperidin-4-yl)-3-methoxy-1H-pyrazol-4-yl)amino)-5-fluoropyrimidin-4-yl)-1H-indole-4-carbonitrile

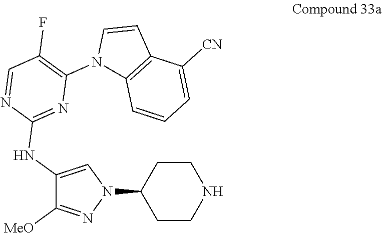

### (S)-1-(5-fluoro-2-((3-methoxy-1-(piperidin-3-yl)-1H-pyrazol-4-yl)amino)pyrimidin-4-yl)-1H-indole-4-carbonitrile

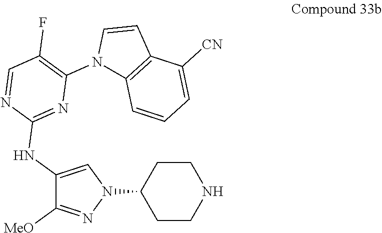

### (R)-1-(5-fluoro-2-((3-methoxy-1-(piperidin-3-yl)-1H-pyrazol-4-yl)amino)pyrimidin-4-yl)-1H-indole-4-carbonitrile

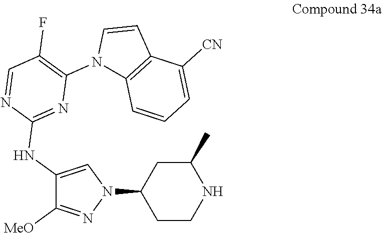 [SMILES: COc1nn([C@@H]2CCN[C@H](C)C2)cc1Nc1ncc(F)c(-n2ccc3c(C#N)cccc32)n1]

### 1-(5-fluoro-2-((3-methoxy-1-((2R,4R)-2-methylpiperidin-4-yl)-1H-pyrazol-4-yl)amino)pyrimidin-4-yl)-1H-indole-4-carbonitrile

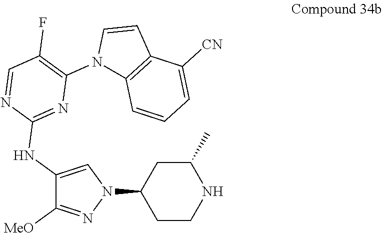 [SMILES: COc1nn([C@@H]2CCN[C@@H](C)C2)cc1Nc1ncc(F)c(-n2ccc3c(C#N)cccc32)n1]

### 1-(5-fluoro-2-((3-methoxy-1-((2S,4R)-2-methylpiperidin-4-yl)-1H-pyrazol-4-yl)amino)pyrimidin-4-yl)-1H-indole-4-carbonitrile

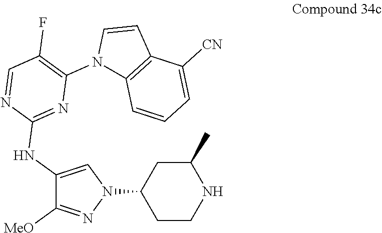 [SMILES: COc1nn([C@H]2CCN[C@H](C)C2)cc1Nc1ncc(F)c(-n2ccc3c(C#N)cccc32)n1]

### 1-(5-fluoro-2-((3-methoxy-1-((2R,4S)-2-methylpiperidin-4-yl)-1H-pyrazol-4-yl)amino)pyrimidin-4-yl)-1H-indole-4-carbonitrile

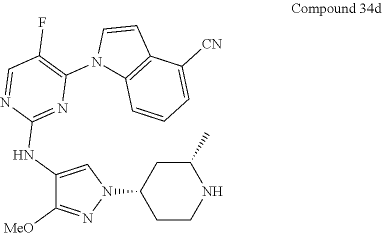 [SMILES: COc1nn([C@H]2CCN[C@@H](C)C2)cc1Nc1ncc(F)c(-n2ccc3c(C#N)cccc32)n1]

### 1-(5-fluoro-2-((3-methoxy-1-((2S,4S)-2-methylpiperidin-4-yl)-1H-pyrazol-4-yl)amino)pyrimidin-4-yl)-1H-indole-4-carbonitrile

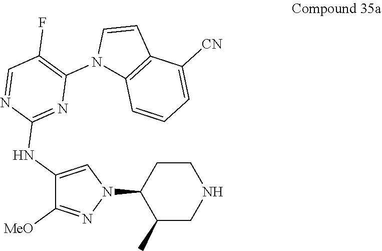 [SMILES: COc1nn([C@H]2CCNC[C@H]2C)cc1Nc1ncc(F)c(-n2ccc3c(C#N)cccc32)n1]

### 1-(5-fluoro-2-((3-methoxy-1-((3R,4S)-3-methylpiperidin-4-yl)-1H-pyrazol-4-yl)amino)pyrimidin-4-yl)-1H-indole-4-carbonitrile

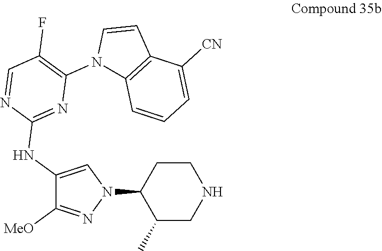 [SMILES: COc1nn([C@H]2CCNC[C@@H]2C)cc1Nc1ncc(F)c(-n2ccc3c(C#N)cccc32)n1]

### 1-(5-fluoro-2-((3-methoxy-1-((3S,4S)-3-methylpiperidin-4-yl)-1H-pyrazol-4-yl)amino)pyrimidin-4-yl)-1H-indole-4-carbonitrile

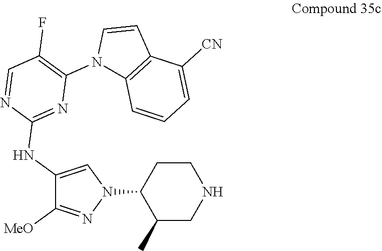 [SMILES: COc1nn([C@@H]2CCNC[C@H]2C)cc1Nc1ncc(F)c(-n2ccc3c(C#N)cccc32)n1]

### 1-(5-fluoro-2-((3-methoxy-1-((3R,4R)-3-methylpiperidin-4-yl)-1H-pyrazol-4-yl)amino)pyrimidin-4-yl)-1H-indole-4-carbonitrile

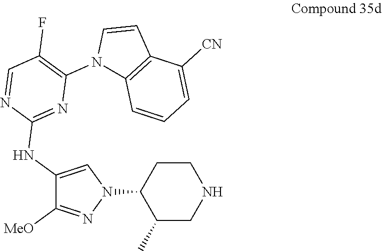 [SMILES: COc1nn([C@@H]2CCNC[C@@H]2C)cc1Nc1ncc(F)c(-n2ccc3c(C#N)cccc32)n1]

### 1-(5-fluoro-2-((3-methoxy-1-((3S,4R)-3-methylpiperidin-4-yl)-1H-pyrazol-4-yl)amino)pyrimidin-4-yl)-1H-indole-4-carbonitrile

### G. Compositions

In one aspect, a composition is provided that includes a macromolecule and a compound described herein. In some aspects, the macromolecule comprises an oligonucleotide. In some aspects, the macromolecule is an antisense oligonucleotide. In some aspects, the macromolecule is a siRNA. In some aspects, the siRNA is conjugated to a lipid and/or a sugar. In some aspects, the siRNA is unconjugated. In some aspects, the macromolecule comprises a polypeptide. In some aspects, the macromolecule comprises one or more components of a SSM system described herein. In some aspects, the SSM system is a CRISPR system. In some aspects, the SSM system is a Cre-Lox system. In some aspects, the SSM system is a FLP-FRT system. In some aspects, the macromolecule comprises a Cas protein and/or a guide RNA. In some aspects, the macromolecule comprises Cre. In some aspects, the macromolecule comprises FLP.

In one aspect, a composition is provided that includes (i) an oligonucleotide and/or polypeptide; and (ii) a compound described herein. In one aspect, a composition is provided that includes (i) an oligonucleotide and/or polypeptide; and (ii) a compound represented by Formula I, or a pharmaceutically acceptable salt thereof. In one aspect, a composition is provided that includes (i) an oligonucleotide and/or polypeptide; and (ii) a compound represented by Formula Ia, or a pharmaceutically acceptable salt thereof. In one aspect, a composition is provided that includes (i) an oligonucleotide and/or polypeptide; and (ii) a compound represented by Formula Ib, or a pharmaceutically acceptable salt thereof. In one aspect, a composition is provided that includes (i) an oligonucleotide and/or polypeptide; and (ii) a compound represented by Formula Ic, or a pharmaceutically acceptable salt thereof. In one aspect, a composition is provided that includes (i) an oligonucleotide and/or polypeptide; and (ii) any one of Compounds 2-35, or a pharmaceutically acceptable salt thereof. In one aspect, a composition is provided that includes (i) an oligonucleotide and/or polypeptide; and (ii) any one of Compounds 4, 8, and 32, or a pharmaceutically acceptable salt thereof. In one aspect, a pharmaceutical composition is provided that includes (i) an oligonucleotide and/or polypeptide, (ii) a compound described herein, and (iii) a pharmaceutically acceptable carrier or diluent. In one aspect, a pharmaceutical composition is provided that includes (i) an oligonucleotide and/or polypeptide, a compound represented by any one of Formulae I, Ia, Ib, and Ic, or a compound listed in Table 1, or a pharmaceutically acceptable salt thereof; and (iii) a pharmaceutically acceptable carrier or diluent. In one aspect, a pharmaceutical composition is provided that includes (i) an oligonucleotide and/or polypeptide; (ii) any one of Compounds 2-35, a pharmaceutically acceptable salt thereof, or combinations thereof; and (iii) a pharmaceutically acceptable carrier or diluent. In one aspect, a pharmaceutical composition is provided that includes (i) an oligonucleotide and/or polypeptide; (ii) any one of Compounds 4, 8, and 32, a pharmaceutically acceptable salt thereof, or combinations thereof, and (iii) a pharmaceutically acceptable carrier or diluent. The pharmaceutical composition can be formulated to be compatible with the intended route of administration, including, but not limited to, parenteral administration, such as intravenous (IV), or subcutaneous (SC or SQ) administration; intraperitoneal, intramuscular, oral, transdermal, or transmucosal administration.

In one aspect, a composition is provided that includes an oligonucleotide. In one aspect, a composition is provided that includes an antisense oligonucleotide (ASO). In one aspect, a composition is provided that includes a splice switching oligonucleotide (SSO). In one aspect, a composition is provided that includes siRNA. In some aspects, the siRNA is conjugated to a lipid and/or a sugar. In some aspects, the siRNA is unconjugated. In one aspect, a composition is provided that includes a guide RNA. In one aspect, a composition is provided that includes about 0.025 μM to about 20 μM oligonucleotide. In one aspect, the composition includes at least about 0.1 μM and up to about 1 μM, about 2 μM, about 3 μM, about 4 μM, about 5 μM, about 6 μM, about 7 μM, about 8 μM, about 9 μM, about 10 μM, about 15 μM, or about 20 μM oligonucleotide. In one aspect, the composition includes from about 0.1 μM to about 10 μM, about 0.1 μM to about 5 μM, or about 0.1 μM to about 1 μM oligonucleotide.

In one aspect, a composition is provided that includes a polypeptide. In one aspect, a composition is provided that includes a Cas protein, e.g., a Cas9 or Cas12a protein. In some aspects, the Cas protein is a modified Cas protein or a Cas fusion protein as described herein. In one aspect, a composition is provided that includes Cre. In one aspect, a composition is provided that includes FLP. In one aspect, a composition is provided that includes about 0.001 μM to about 20 μM polypeptide. In one aspect, the composition includes at least about 0.01 μM and up to about 1 μM, about 2 μM, about 3 μM, about 4 μM, about 5 μM, about 6 μM, about 7 μM, about 8 μM, about 9 μM, about 10 μM, about 15 μM, or about 20 μM polypeptide. In one aspect, the composition includes from about 0.01 μM to about 10 μM, about 0.05 μM to about 5 μM, or about 0.1 μM to about 1 μM polypeptide.

In one aspect, a composition is provided that includes an oligonucleotide and a polypeptide. In one aspect, a composition is provided that includes a Cas protein and a guide RNA. In some aspects, the Cas protein is Cas9. In some aspects, the Cas protein is Cas12a. In some aspects, the Cas protein is a modified Cas protein or a Cas fusion protein as described herein. In one aspect, a composition is provided that includes about 0.001 μM to about 20 μM of each of the oligonucleotide and the polypeptide. In one aspect, the composition includes at least about 0.1 μM and up to about 1 μM, about 2 μM, about 3 μM, about 4 μM, about 5 μM, about 6 μM, about 7 μM, about 8 μM, about 9 μM, about 10 μM, about 15 μM, or about 20 μM of each of the oligonucleotide and the polypeptide. In one aspect, the composition includes from about 0.01 μM to about 10 μM, about 0.05 μM to about 5 μM, or about 0.1 μM to about 1 μM of each of the oligonucleotide and the polypeptide.

In one aspect, a composition is provided that includes a compound described herein. In one aspect, a composition is provided that includes a small molecule compound described herein. In one aspect, a composition is provided that includes a compound represented by Formulae I, Ia, Ib, or Ic, or the compounds listed in Table 1, or a pharmaceutically acceptable salt thereof. In one aspect, a composition is provided that includes a compound represented by any one of Formulae I, Ia, Ib, or Ic, or the compounds listed in Table 1, a pharmaceutically acceptable salt thereof, or combinations thereof. In one aspect, a composition is provided that includes any one of Compounds 4, 8, and 32, or a pharmaceutically acceptable salt thereof. In one aspect, a composition is provided that includes a compound of any one of Compounds 4, 8, and 32, a pharmaceutically acceptable salt thereof, or combinations thereof. In one aspect, a composition is provided includes about 1 μM to about 20 μM of a compound described herein. In one aspect, the composition includes about 1 μM to about 10 μM of a compound described herein. In one aspect, the composition includes about 1 μM to about 5 μM of a compound described herein. In one aspect, the composition includes at least about 1 μM, about 1.5 μM, about 2 μM, about 2.5 μM, about 3 μM, about 3.5 μM, about 4 μM, about 4.5 μM, or about 5 μM and up to about 6 μM, about 6.5 μM, about 7 μM, about 7.5 μM, about 8 μM, about 8.5 μM, about 9 μM, about 9.5 μM, about 10 μM, about 15 μM or about 20 μM of a compound described herein.

In one aspect, a composition is provided that includes about 0.025 μM to about 20 μM oligonucleotide and about 1 μM to about 20 μM of a compound described herein. In one aspect, the composition includes at least about 0.1 μM and up to about 1 μM, about 2 μM, about 3 μM, about 4 μM, about 5 μM, about 6 μM, about 7 μM, about 8 μM, about 9 μM, about 10 μM, about 15 μM, or about 20 μM oligonucleotide and at least about 1 μM, about 1.5 μM, about 2 μM, about 2.5 μM, about 3 μM, about 3.5 μM, about 4 μM, about 4.5 μM, or about 5 μM and up to about 6 μM, about 6.5 μM, about 7 μM, about 7.5 μM, about 8 μM, about 8.5 μM, about 9 μM, about 9.5 μM, about 10 μM, about 15 μM or about 20 μM of a compound described herein. In one aspect, the composition includes from about 0.1 μM to about 10 μM, about 0.1 μM to about 5 μM, or about 0.1 μM to about 1 μM oligonucleotide and about 1 μM to about 1 μM to about 20 μM, about 1 μM to about 10 μM, or about 1 μM to about 5 μM of a compound described herein. In some aspects, the oligonucleotide is ASO. In some aspects, the oligonucleotide is SSO. In some aspects, the oligonucleotide is siRNA. In some aspects, the siRNA is conjugated to a lipid and/or a sugar. In some aspects, the siRNA is unconjugated. In some aspects, the oligonucleotide is a guide RNA.

In one aspect, a composition is provided that includes about 0.001 μM to about 20 μM polypeptide and about 1 μM to about 20 μM of a compound described herein. In one aspect, the composition includes at least about 0.01 μM and up to about 1 μM, about 2 μM, about 3 μM, about 4 μM, about 5 μM, about 6 μM, about 7 μM, about 8 μM, about 9 μM, about 10 μM, about 15 μM, or about 20 μM polypeptide and at least about 1 μM, about 1.5 μM, about 2 μM, about 2.5 μM, about 3 μM, about 3.5 μM, about 4 μM, about 4.5 μM, or about 5 μM and up to about 6 μM, about 6.5 μM, about 7 μM, about 7.5 μM, about 8 μM, about 8.5 μM, about 9 μM, about 9.5 μM, about 10 μM, about 15 μM or about 20 μM of a compound described herein. In one aspect, the composition includes from about 0.01 μM to about 10 μM, about 0.05 μM to about 5 μM, or about 0.1 μM to about 1 μM polypeptide and about 1 μM to about 1 μM to about 20 μM, about 1 μM to about 10 μM, or about 1 μM to about 5 μM of a compound described herein. In some aspects, the polypeptide is a Cas protein. In some aspects, the polypeptide is a recombinase. In some aspects, the polypeptide is Cre. In some aspects, the polypeptide is FLP.

In one aspect, a composition is provided that includes about 0.001 μM to about 20 μM of each of an oligonucleotide and a polypeptide, and about 1 μM to about 20 μM of a compound described herein. In one aspect, the composition includes at least about 0.01 μM and up to about 1 μM, about 2 μM, about 3 μM, about 4 μM, about 5 μM, about 6 μM, about 7 μM, about 8 μM, about 9 μM, about 10 μM, about 15 μM, or about 20 μM of each of an oligonucleotide and a polypeptide, and at least about 1 μM, about 1.5 μM, about 2 μM, about 2.5 μM, about 3 μM, about 3.5 μM, about 4 μM, about 4.5 μM, or about 5 μM and up to about 6 μM, about 6.5 μM, about 7 μM, about 7.5 μM, about 8 μM, about 8.5 μM, about 9 μM, about 9.5 μM, about 10 μM, about 15 μM or about 20 μM of a compound described herein. In one aspect, the composition includes from about 0.01 μM to about 10 μM, about 0.05 μM to about 5 μM, or about 0.1 μM to about 1 μM of each of an oligonucleotide and a polypeptide, and about 1 μM to about 1 μM to about 20 μM, about 1 μM to about 10 μM, or about 1 μM to about 5 μM of a compound described herein. In some aspects, the polypeptide is a Cas protein, and the oligonucleotide is a guide RNA.

The “compositions comprising the oligonucleotides and/or polypeptides” and/or the “compositions comprising the compounds of Formulae I, Ia, Ib, or Ic, or the compounds listed in Table 1” can include a biological cell, e.g., an in vitro cell. For example, the compositions comprising the oligonucleotides, polypeptides, and/or the compounds of Formula I, Formula Ia, Formula Ib, or Formula Ic, or the compounds listed in Table 1 can include an in vitro cell culture, e.g., a mammalian cell culture, and the concentrations described above can refer to the concentration of the oligonucleotide, polypeptide, and/or compounds of Formula I, Formula Ia, Formula Ib, or Formula Ic, or the compounds listed in Table 1 in the cell culture. In another aspect, the compositions comprising the oligonucleotides, polypeptides, and/or the compounds of Formula I, Formula Ia, Formula Ib, or Formula Ic, or the compounds listed in Table 1 can include an in vitro isolated cell from a subject, e.g., a mammalian subject, e.g., a human subject, and the concentrations described above can refer to the concentration of the oligonucleotide, polypeptide, and/or compounds of Formula I, Formula Ia, Formula Ib, or Formula Ic, or the compounds listed in Table 1 in combination with the isolated cell from the subject.

### H. Methods of Treatment

In one aspect, a method is provided for introducing a macromolecule into a cell, e.g., a target cell described herein. In one aspect, the cell is a cultured cell. In one aspect, the cell is an isolated cell. In one aspect, the cell is an isolated cell from a subject in need of treatment. In one aspect, the cell is a mammalian cell. In one aspect, the cell is a eukaryotic cell and/or a prokaryotic cell.

In one aspect, the cell is part of a tissue or organ of a mammal, e.g., a human. In one aspect, the organ or tissue is the brain, central nervous system (CNS) or peripheral nervous system (PNS), heart, liver, kidney, spleen, pancreas, lung, adipose, and/or muscle (e.g., skeletal muscle). In one aspect, the cell is a brain cell, a CNS cell, a PNS cell, a heart cell, a liver cell, a kidney cell, a spleen cell, a pancreas cell, a lung cell, a muscle cell, an adipose cell, an immune cell, or combination thereof.

In some aspects, the cell is a CNS cell. In some aspects, the CNS cell comprises a glial cell and/or a neuron. Glial cells of the CNS include, e.g., astrocytes, oligodendrocytes, microglia, and ependymal cells. Neurons include, e.g., afferent neurons, efferent neurons, and interneurons.

In some aspects, the cell is a liver cell, e.g., a hepatocyte or a non-parenchymal cell. In some aspects, the cell comprises a plateable metabolism qualified human hepatocyte, a plateable induction qualified human hepatocyte, plateable human hepatocyte, suspension qualified human hepatocyte (including 10-donor and 20-donor pooled hepatocytes), human hepatic Kupffer cells, human hepatic stellate cells, dog hepatocytes (including single and pooled Beagle hepatocytes), mouse hepatocytes (including CD-1 and C57BI/6 hepatocytes), rat hepatocytes (including Sprague-Dawley, Wistar Han, and Wistar hepatocytes), monkey hepatocytes (including Cynomolgus or Rhesus monkey hepatocytes), cat hepatocytes (including Domestic Shorthair hepatocytes), and rabbit hepatocytes (including New Zealand White hepatocytes).

In some aspects, the cell is a human stem cell. The stem cells can be, for example, pluripotent stem cells, including embryonic stem cells (ESCs), adult stem cells, induced pluripotent stem cells (iPSCs), tissue specific stem cells (e.g., hematopoietic stem cells) and mesenchymal stem cells (MSCs). In some aspects, the cell is a differentiated form of any of the cells described herein. In some aspects, the eukaryotic cell is a cell derived from any primary cell in culture.

In some aspects, the cell is an immune cell. Non-limiting examples of immune cells include T cells, B cells, dendritic cells, NK cells, T helper cells, cytotoxic T cells, regulatory T cells, gamma delta T cells, neutrophils, mast cells, monocytes, antigen-presenting cells, lymphocytes, basophils, and phagocytes.

In one aspect, a method is provided for introducing an oligonucleotide into a cell. In one aspect, a method is provided for introducing a polypeptide into a cell. In one aspect, a method is provided for introducing an oligonucleotide into a nucleus and/or cytosol of a cell. In one aspect, a method is provided for introducing a polypeptide into a nucleus and/or cytosol of a cell. In one aspect, the oligonucleotide is ASO, SSO, and/or siRNA. In one aspect, the oligonucleotide is siRNA, wherein the siRNA is unconjugated. In one aspect, the oligonucleotide is siRNA, wherein the siRNA is conjugated to a lipid and/or a sugar. In one aspect, the oligonucleotide is a guide RNA. In one aspect, the oligonucleotide hybridizes to a target nucleic acid in the cell. In one aspect, the polypeptide is capable of providing a SSM in a target nucleic acid in the cell. In one aspect, the target nucleic acid is in the nucleus of the cell. In one aspect, the target nucleic acid is in cytosol of the cell.

In one aspect, the method includes: (a) contacting the cell with the oligonucleotide and/or polypeptide; and (b) contacting the cell with a compound described herein. In one aspect, the oligonucleotide is an antisense oligonucleotide (ASO). In one aspect, the oligonucleotide is a splice switching oligonucleotide (SSO). In one aspect, the oligonucleotide is siRNA. In some aspects, the siRNA is conjugated to a lipid and/or a sugar. In some aspects, the siRNA is unconjugated. In one aspect, the cell is contacted with an oligonucleotide. In one aspect, the cell is contacted with a polypeptide. In one aspect, the cell is contacted with both an oligonucleotide and a polypeptide. In one aspect, the oligonucleotide is a guide RNA. In one aspect, the polypeptide is a recombinase. In one aspect, the polypeptide is Cre. In one aspect, the polypeptide is FLP. In one aspect, the polypeptide is a Cas protein. In one aspect, the polypeptide is a Cas protein and the oligonucleotide is a guide RNA. In one aspect, the compound is a small molecule compound (SMC). In one aspect, the compound is an endosomolytic compound. In one aspect, the compound has a structure represented by Formula I, or a pharmaceutically acceptable salt thereof. In one aspect, the compound has a structure represented by Formula Ia, or a pharmaceutically acceptable salt thereof. In one aspect, the compound has a structure represented by Formula Ib, or a pharmaceutically acceptable salt thereof. In one aspect, the compound has a structure represented by Formula Ic, or a pharmaceutically acceptable salt thereof. In one aspect, the compound has a structure represented by Formula I, Formula Ia, Formula Ib, or Formula Ic, or the compounds listed in Table 1, or a pharmaceutically acceptable salt thereof. In one aspect, the compound is any one of Compounds 4, 8, and 32, or a pharmaceutically acceptable salt thereof.

In one aspect, the method includes in vitro delivery of the macromolecule, e.g., oligonucleotide and/or polypeptide, and/or the compound to the cell. In one aspect, the method includes in vivo delivery of the macromolecule, e.g., oligonucleotide and/or polypeptide, and/or the compound to the cell. In one aspect, the cell is a cultured cell. In one aspect, the cell is an isolated cell from a patient in need of treatment. In one aspect, the cell is part of a tissue or organ. In one aspect, the cell is a mammalian cell. In one aspect, the cell is a human cell. In one aspect the cell is a eukaryotic and/or a prokaryotic cell.

In one aspect, the macromolecule, e.g., oligonucleotide and/or polypeptide, is internalized by the cell through endocytosis and encapsulated within an endosome. In one aspect, the compound facilitates release of the macromolecule, e.g., oligonucleotide and/or polypeptide, from the endosome.

In one aspect, the macromolecule, e.g., oligonucleotide and/or polypeptide, is internalized by the cell through transient pore formation. In one aspect, the compound facilitates transient pore formation in the plasma membrane.

In one aspect, the cell is contacted with a composition that includes an oligonucleotide. In one aspect, the cell is contacted with a composition that includes an antisense oligonucleotide (ASO). In one aspect, the cell is contacted with a composition that includes a splice switching oligonucleotide (SSO). In one aspect, the cell is contacted with a composition that includes a siRNA. In some aspects, the siRNA is conjugated to a lipid and/or a sugar. In some aspects, the siRNA is unconjugated. In one aspect, the cell is contacted with a composition that includes a guide RNA. In one aspect, the cell is contacted with a composition that includes oligonucleotide in an amount sufficient to provide a therapeutic effect. In one aspect, the cell is contacted with a composition that includes oligonucleotide in an amount sufficient to provide a SSM at a target nucleic acid. In one aspect, the cell is contacted with a composition that includes about 0.025 μM to about 20 μM oligonucleotide. In one aspect, the cell is contacted with a composition that includes at least about 0.1 μM and up to about 1 μM, about 2 μM, about 3 μM, about 4 μM, about 5 μM, about 6 μM, about 7 μM, about 8 μM, about 9 μM, about 10 μM, about 15 μM, or about 20 μM, oligonucleotide. In one aspect, the cell is contacted with a composition that includes from about 0.1 μM to about 10 μM, about 0.1 μM to about 5 μM, or about 0.1 μM to about 1 μM oligonucleotide.

In one aspect, the cell is contacted with a composition that includes a polypeptide. In one aspect, the cell is contacted with a composition that includes a Cas protein. In one aspect, the cell is contacted with a composition that includes a recombinase, e.g., Cre or FLP. In one aspect, the cell is contacted with a composition that includes polypeptide in an amount sufficient to provide a therapeutic effect. In one aspect, the cell is contacted with a composition that includes polypeptide in an amount sufficient to provide a SSM at a target nucleic acid. In one aspect, the cell is contacted with a composition that includes about 0.3 μM to about 20 μM polypeptide. In one aspect, the cell is contacted with a composition that includes at least about 0.01 μM and up to about 1 μM, about 2 μM, about 3 μM, about 4 μM, about 5 μM, about 6 μM, about 7 μM, about 8 μM, about 9 μM, about 10 μM, about 15 μM, or about 20 μM, polypeptide. In one aspect, the cell is contacted with a composition that includes from about 0.01 μM to about 10 μM, about 0.05 μM to about 5 μM, or about 0.1 μM to about 1 μM polypeptide.

In one aspect, the cell is contacted with a composition that includes an oligonucleotide and a polypeptide. In one aspect, the cell is contacted with a composition that includes a Cas protein and a guide RNA. In one aspect, the cell is contacted with a composition that includes oligonucleotide and polypeptide in an amount sufficient to provide a therapeutic effect. In one aspect, the cell is contacted with a composition that includes oligonucleotide and polypeptide in an amount sufficient to provide a SSM at a target nucleic acid. In one aspect, the cell is contacted with a composition that includes about 0.001 μM to about 20 μM of each of an oligonucleotide (e.g., guide RNA) and a polypeptide (e.g., Cas protein). In one aspect, the cell is contacted with a composition that includes at least about 0.01 μM and up to about 1 μM, about 2 μM, about 3 μM, about 4 μM, about 5 μM, about 6 μM, about 7 μM, about 8 μM, about 9 μM, about 10 μM, about 15 μM, or about 20 μM of each of the oligonucleotide and polypeptide. In one aspect, the cell is contacted with a composition that includes from about 0.01 μM to about 10 μM, about 0.05 μM to about 5 μM, or about 0.1 μM to about 1 μM of each of the oligonucleotide and polypeptide.

In one aspect, the cell is contacted with a composition that includes a compound described herein. In one aspect, the cell is contacted with a composition that includes a compound represented by Formula I or a pharmaceutically acceptable salt thereof. In one aspect, the cell is contacted with a composition that includes a compound represented by Formula I, Formula Ia, Formula Ib, or Formula Ic, or the compounds listed in Table 1, a pharmaceutically acceptable salt thereof, or combinations thereof. In one aspect, the cell is contacted with a composition that includes any one of Compounds 4, 8, and 32, a pharmaceutically acceptable salt thereof, or combinations thereof. In one aspect, the cell is contacted with a composition at a concentration sufficient to (i) reduce accumulation of the oligonucleotide in endosomes and/or lysosomes, (ii) induce physical rupture of endosomes, and/or (iii) facilitate transient pore formation in the plasma membrane, thereby facilitating release of a macromolecule, e.g., an oligonucleotide and/or polypeptide, into the cytosol or nucleus.

In one aspect, the cell is contacted with a composition that includes about 1 μM to about 20 μM of a compound described herein. In one aspect, the cell is contacted with a composition that includes about 1 μM to about 10 μM a compound described herein. In one aspect, the cell is contacted with a composition that includes about 1 μM to about 5 μM a compound described herein. In one aspect, the cell is contacted with a composition that includes at least about 1 μM, about 1.5 μM, about 2 μM, about 2.5 μM, about 3 μM, about 3.5 μM, about 4 μM, about 4.5 μM, or about 5 μM and up to about 6 μM, about 6.5 μM, about 7 μM, about 7.5 μM, about 8 μM, about 8.5 μM, about 9 μM, about 9.5 μM, about 10 μM, about 15 μM or about 20 μM of a compound described herein. In some embodiments, the compound has a structure represented by Formula I, Formula Ia, Formula Ib, or Formula Ic, or is a compound listed in Table 1, or a pharmaceutically acceptable salt thereof, or combinations thereof. In some embodiments, the compound is any one of Compounds 4, 8, and 32, a pharmaceutically acceptable salt thereof, or combinations thereof.

In one aspect, the cell is contacted with a composition that includes about 0.025 μM to about 20 μM oligonucleotide and about 1 μM to about 20 μM of a compound described herein. In one aspect, the cell is contacted with a composition that includes at least about 0.1 μM and up to about 1 μM, about 2 μM, about 3 μM, about 4 μM, about 5 μM, about 6 μM, about 7 μM, about 8 μM, about 9 μM, about 10 μM, about 15 μM, or about 20 μM, oligonucleotide and at least about 1 μM, about 1.5 μM, about 2 μM, about 2.5 μM, about 3 μM, about 3.5 μM, about 4 μM, about 4.5 μM, or about 5 μM and up to about 6 μM, about 6.5 μM, about 7 μM, about 7.5 μM, about 8 μM, about 8.5 μM, about 9 μM, about 9.5 μM, about 10 μM, about 15 μM or about 20 μM of a compound described herein. In one aspect, the cell is contacted with a composition that includes from about 0.1 μM to about 10 μM, about 0.1 μM to about 5 μM, or about 0.1 μM to about 1 μM oligonucleotide and about 1 μM to about 1 μM to about 20 μM, about 1 μM to about 10 μM, or about 1 μM to about 5 μM of a compound described herein. In one aspect, the composition includes an oligonucleotide. In one aspect, the composition includes an antisense oligonucleotide (ASO). In one aspect, the composition includes a splice switching oligonucleotide (SSO). In one aspect, the composition includes siRNA. In some aspects, the siRNA is conjugated to a lipid and/or a sugar. In some aspects, the siRNA is unconjugated. In one aspect, the composition includes a guide RNA. In one aspect, the composition includes a compound described herein. In one aspect, the composition includes a compound represented by Formula I or a pharmaceutically acceptable salt thereof. In one aspect, the composition includes a compound represented by Formula I, Formula Ia, Formula Ib, or Formula Ib, or the compounds listed in Table 1, a pharmaceutically acceptable salt thereof, or combinations thereof. In one aspect, the compound is any one of Compounds 4, 8, and 32, a pharmaceutically acceptable salt thereof, or combinations thereof.

In one aspect, the cell is contacted with a composition that includes about 0.025 μM to about 20 μM polypeptide and about 1 μM to about 20 μM of a compound described herein. In one aspect, the cell is contacted with a composition that includes at least about 0.1 μM and up to about 1 μM, about 2 μM, about 3 μM, about 4 μM, about 5 μM, about 6 μM, about 7 μM, about 8 μM, about 9 μM, about 10 μM, about 15 μM, or about 20 μM, polypeptide and at least about 1 μM, about 1.5 μM, about 2 μM, about 2.5 μM, about 3 μM, about 3.5 μM, about 4 μM, about 4.5 μM, or about 5 μM and up to about 6 μM, about 6.5 μM, about 7 μM, about 7.5 μM, about 8 μM, about 8.5 μM, about 9 μM, about 9.5 μM, about 10 μM, about 15 μM or about 20 μM of a compound described herein. In one aspect, the cell is contacted with a composition that includes from about 0.1 μM to about 10 μM, about 0.1 μM to about 5 μM, or about 0.1 μM to about 1 μM polypeptide and about 1 μM to about 1 μM to about 20 μM, about 1 μM to about 10 μM, or about 1 μM to about 5 μM of a compound described herein.

In one aspect, the composition includes a polypeptide. In one aspect, the composition includes a Cas protein, e.g., Cas9 or Cas12a. In one aspect, the composition includes a recombinase, e.g., Cre or FLP. In one aspect, the composition includes a compound described herein. In one aspect, the composition includes a compound represented by Formula I or a pharmaceutically acceptable salt thereof. In one aspect, the composition includes a compound represented by Formula I, Formula Ia, Formula Ib, or Formula Ib, or the compounds listed in Table 1, a pharmaceutically acceptable salt thereof, or combinations thereof. In one aspect, the compound is any one of Compounds 4, 8, and 32, a pharmaceutically acceptable salt thereof, or combinations thereof.

In one aspect, the cell is contacted with a composition that includes about 0.025 μM to about 20 μM of each of an oligonucleotide and a polypeptide, and about 1 μM to about 20 μM of a compound described herein. In one aspect, the cell is contacted with a composition that includes at least about 0.1 μM and up to about 1 μM, about 2 μM, about 3 μM, about 4 μM, about 5 μM, about 6 μM, about 7 μM, about 8 μM, about 9 μM, about 10 μM, about 15 μM, or about 20 μM of each of an oligonucleotide and a polypeptide, and at least about 1 μM, about 1.5 μM, about 2 μM, about 2.5 μM, about 3 μM, about 3.5 μM, about 4 μM, about 4.5 μM, or about 5 μM and up to about 6 μM, about 6.5 μM, about 7 μM, about 7.5 μM, about 8 μM, about 8.5 μM, about 9 μM, about 9.5 μM, about 10 μM, about 15 μM or about 20 μM of a compound described herein. In one aspect, the cell is contacted with a composition that includes from about 0.1 μM to about 10 μM, about 0.1 μM to about 5 μM, or about 0.1 μM to about 1 μM of each of an oligonucleotide and a polypeptide, and about 1 μM to about 1 μM to about 20 μM, about 1 μM to about 10 μM, or about 1 μM to about 5 μM of a compound described herein. In one aspect, the composition includes an oligonucleotide and a polypeptide. In one aspect, the composition includes a Cas protein, e.g., Cas9 or Cas12a, and a guide RNA. In one aspect, the composition includes a compound described herein. In one aspect, the composition includes a compound represented by Formula I or a pharmaceutically acceptable salt thereof. In one aspect, the composition includes a compound represented by Formula I, Formula Ia, Formula Ib, or Formula Ib, or the compounds listed in Table 1, a pharmaceutically acceptable salt thereof, or combinations thereof. In one aspect, the compound is any one of Compounds 4, 8, and 32, a pharmaceutically acceptable salt thereof, or combinations thereof.

In one aspect, the cell is contacted with the macromolecule, e.g., the oligonucleotide and/or the polypeptide, and the compound concurrently, i.e., at approximately the same time. In one aspect, the cell is contacted with a composition that includes the macromolecule, e.g., the oligonucleotide and/or the polypeptide, and the compound. In one aspect, the macromolecule, e.g., the oligonucleotide and/or the polypeptide, and the compound are in the same composition. In one aspect, the cell is contacted with a first composition that includes the macromolecule, e.g., the oligonucleotide and/or the polypeptide, and a second composition that includes the compound. In one aspect, the cell is contacted with a first composition that includes the macromolecule, e.g., the oligonucleotide and/or the polypeptide, and a second composition that includes the compound at approximately the same time. In one aspect, the cell is contacted with the macromolecule, e.g., the oligonucleotide and/or the polypeptide, and the compound within less than about 6 hours, about 3 hours, about 2 hours, about 1 hour, about 30 minutes, or about 15 minutes of each other.

In one aspect, the cell is contacted with the macromolecule, e.g., the oligonucleotide and/or the polypeptide, and the compound sequentially. In one aspect, the cell is contacted with the macromolecule, e.g., the oligonucleotide and/or the polypeptide, before it is contacted with the compound. In one aspect, the cell is contacted with the macromolecule, e.g., the oligonucleotide and/or the polypeptide, after it is contacted with the compound. In one aspect, the cell is contacted with a first composition that includes the macromolecule, e.g., the oligonucleotide and/or the polypeptide, before it is contacted with a second composition that includes the compound. In one aspect, the cell is contacted with the macromolecule, e.g., the oligonucleotide and/or the polypeptide, up to about 48 hours before the cell is contacted with the compound. In one aspect, the cell is contacted with the macromolecule, e.g., the oligonucleotide and/or the polypeptide, from about 12 hours to about 48 hours before the cell is contacted with the compound. In one aspect, the cell is contacted with the macromolecule, e.g., the oligonucleotide and/or the polypeptide, at least about 12 hours or about 24 hours and up to about 36 hours or about 48 hours before the cell is contacted with the compound.

In one aspect, contacting the cell with a compound described herein results in endosomal membrane permeabilization as determined by mCherry-GAL9 recruitment assay. In one aspect, contacting the cell with a compound with a structure shown in Formula I, or a pharmaceutically acceptable salt thereof results in endosomal membrane permeabilization as determined by mCherry-GAL9 recruitment assay. In one aspect, contacting the cell with a compound with a structure shown in Formula I, Formula Ia, Formula Ib, or Formula Ic, or the compounds listed in Table 1, or a pharmaceutically acceptable salt thereof, results in membrane permeabilization as determined by mCherry-GAL9 recruitment assay. In one aspect, contacting the cell with a compound of any one of Compounds 2-35, or a pharmaceutically acceptable salt thereof, results in membrane permeabilization as determined by mCherry-GAL9 recruitment assay. In one aspect, contacting the cell with a compound with the structure of Compound 2, or a pharmaceutically acceptable salt thereof, results in membrane permeabilization as determined by mCherry-GAL9 recruitment assay. In one aspect, contacting the cell with a compound with the structure of Compound 4, or a pharmaceutically acceptable salt thereof, results in membrane permeabilization as determined by mCherry-GAL9 recruitment assay. In one aspect, contacting the cell with a compound with the structure of Compound 8, or a pharmaceutically acceptable salt thereof, results in membrane permeabilization as determined by mCherry-GAL9 recruitment assay. In one aspect, contacting the cell with a compound with the structure of Compound 32, or a pharmaceutically acceptable salt thereof, results in membrane permeabilization as determined by mCherry-GAL9 recruitment assay. In one aspect, contacting the cell with a compound with a structure shown in Formula I, Formula Ia, Formula Ib, or Formula Ic, or the compounds listed in Table 1, or a pharmaceutically acceptable salt thereof, results in endosomal membrane permeabilization as determined by mCherry-GAL9 recruitment assay.

In one aspect, the macromolecule, e.g., oligonucleotide and/or polypeptide, alters activity of a gene expressed by the cell. In one aspect, the gene is an endogenous gene. In one aspect, the gene is an exogenous gene. In one aspect, the macromolecule, e.g., oligonucleotide and/or polypeptide, increases activity of a gene expressed by the cell. In one aspect, the activity of the gene expressed by the cell is increased at least about 10× when the cell is contacted with the macromolecule, e.g., oligonucleotide and/or polypeptide, and the compound as compared to a cell that is contacted with the macromolecule, e.g., oligonucleotide and/or polypeptide, and not the compound. In one aspect, the activity of the gene expressed by the cell is increased at least about 10×, about 20×, about 30×, about 40× or about 50× and up to about 100×, 200×, 300×, 400×, 500× or 1000×. In one aspect, the activity of the gene expressed by the cell is increased from about 10× to about 1000×, about 10× to about 500×, about 10× to about 400×, about 10× to about 300×, about 10× to about 200×, about 10× to about 100×, about 100× to about 300×, or about 100× to about 200×. In one aspect, the activity of the gene expressed by a cell is increased from about 10× to about 1000× when the cell is contacted with the macromolecule, e.g., oligonucleotide and/or polypeptide, and the compound as compared to a cell that is contacted with the macromolecule, e.g., oligonucleotide and/or polypeptide, and not the compound. In one aspect, the activity of the gene expressed by a cell is increased from about 100× to about 500×. In one aspect, the activity of the gene expressed by a cell is increased from about 100× to about 300×. In one aspect, the activity of the gene expressed by a cell is increased from about 100× to about 200×. In one aspect, the activity of the gene expressed by a cell is increased from about 10× to about 500×. In one aspect, the activity of the gene expressed by a cell is increased from about 10× to about 300×. In one aspect, the activity of the gene expressed by a cell is increased from about 10× to about 200×. In one aspect, the activity of the gene expressed by a cell is increased from about 10× to about 100×. In one aspect, the activity of the gene expressed by a cell is increased from about 10× to about 50×.

In one aspect, the macromolecule, e.g., oligonucleotide and/or polypeptide, decreases the activity of a gene expressed by the cell. In one aspect, the activity of the gene expressed by the cell is decreased at least about 10× when the cell is contacted with the macromolecule, e.g., oligonucleotide and/or polypeptide, and the compound as compared to a cell that is contacted with the macromolecule, e.g., oligonucleotide and/or polypeptide, and not the compound. In one aspect, the activity of the gene expressed by the cell is decreased at least about 10×, about 20×, about 30×, about 40× or about 50× and up to about 100×, 200×, 300×, 400×, 500× or 1000×. In one aspect, the activity of the gene expressed by the cell is decreased from about 10× to about 1000×, about 10× to about 500×, about 10× to about 400×, about 10× to about 300×, about 10× to about 200×, about 10× to about 100×, about 100× to about 300×, or about 100× to about 200×. In one aspect, the activity of the gene expressed by a cell is decreased from about 10× to about 1000× when the cell is contacted with the macromolecule, e.g., oligonucleotide and/or polypeptide, and the compound as compared to a cell that is contacted with the macromolecule, e.g., oligonucleotide and/or polypeptide, and not the compound. In one aspect, the activity of the gene expressed by a cell is decreased from about 100× to about 500×. In one aspect, the activity of the gene expressed by a cell is decreased from about 100× to about 300×. In one aspect, the activity of the gene expressed by a cell is decreased from about 100× to about 200×. In one aspect, the activity of the gene expressed by a cell is decreased from about 10× to about 500×. In one aspect, the activity of the gene expressed by a cell is decreased from about 10× to about 300×. In one aspect, the activity of the gene expressed by a cell is decreased from about 10× to about 200×. In one aspect, the activity of the gene expressed by a cell is decreased from about 10× to about 100×. In one aspect, the activity of the gene expressed by a cell is decreased from about 10× to about 50×.

In one aspect, a method is provided for altering expression of a target nucleic acid in a cell. In one aspect, the method includes: contacting the cell with an oligonucleotide that is capable of hybridizing to the target nucleic acid, wherein the oligonucleotide is internalized by the cell through endocytosis and encapsulated within an endosome; and contacting the cell with a compound described herein, wherein the compound facilitates release of the oligonucleotide from the endosome, wherein the hybridization of the oligonucleotide to the target nucleic acid alters expression of the target nucleic acid. In one aspect, hybridization of the oligonucleotide to the target nucleic acid increases expression of the target nucleic acid. In one aspect, hybridization of the oligonucleotide to the target nucleic acid decreases expression of the target nucleic acid. In some embodiments, the oligonucleotide is an ASO. In some embodiments, the oligonucleotide is an SSO. In some embodiments, the oligonucleotide is an siRNA. In some embodiments, the oligonucleotide is conjugated to a lipid and/or a sugar and/or a peptide. In some aspects, the oligonucleotide is unconjugated. In some aspects, the combination of an unconjugated oligonucleotide and the compound provides similar levels of altered expression of a target nucleic acid as compared to a oligonucleotide conjugated to a lipid and/or a sugar and/or a peptide. In some embodiments, the compound has a structure represented by Formula I, Formula Ia, Formula Ib, or Formula Ic, or is a compound listed in Table 1, or a pharmaceutically acceptable salt thereof, or combinations thereof. In one aspect, the compound is any one of Compounds 4, 8, and 32, a pharmaceutically acceptable salt thereof, or combinations thereof.

In one aspect, a method is provided for altering expression of a target nucleic acid in a cell. In one aspect, the method includes: contacting the cell with a polypeptide that is capable of performing a SSM at the target nucleic acid, wherein the polypeptide is internalized by the cell through endocytosis and encapsulated within an endosome; and contacting the cell with a compound described herein, wherein the compound facilitates release of the polypeptide from the endosome, wherein the SSM at the target nucleic acid alters expression of the target nucleic acid. In one aspect, the SSM increases expression of the target nucleic acid. In one aspect, the SSM decreases expression of the target nucleic acid. In some embodiments, the polypeptide is a Cas protein. In some embodiments, the polypeptide is a recombinase/meganuclease. In some embodiments, the polypeptide is Cre or FLP. In some embodiments, the compound has a structure represented by Formula I, Formula Ia, Formula Ib, or Formula Ic, or is a compound listed in Table 1, or a pharmaceutically acceptable salt thereof, or combinations thereof. In one aspect, the compound is any one of Compounds 4, 8, and 32, a pharmaceutically acceptable salt thereof, or combinations thereof.

In one aspect, a method is provided for altering expression of a target nucleic acid in a cell. In one aspect, the method includes: contacting the cell with a polypeptide and an oligonucleotide, wherein the polypeptide is capable of performing a SSM and the oligonucleotide is capable of hybridizing to the target nucleic acid, wherein the polypeptide and the oligonucleotide are internalized by the cell through endocytosis and encapsulated within an endosome; and contacting the cell with a compound described herein, wherein the compound facilitates release of the polypeptide and the oligonucleotide from the endosome, wherein the hybridization of the oligonucleotide to the target nucleic acid enables the polypeptide to perform the SSM, thereby altering expression of the target nucleic acid. In one aspect, the SSM increases expression of the target nucleic acid. In one aspect, the SSM decreases expression of the target nucleic acid. In some embodiments, the oligonucleotide is a guide RNA, and the polypeptide is a Cas protein. In some embodiments, the compound has a structure represented by Formula I, Formula Ia, Formula Ib, or Formula Ic, or is a compound listed in Table 1, or a pharmaceutically acceptable salt thereof, or combinations thereof. In one aspect, the compound is any one of Compounds 4, 8, and 32, a pharmaceutically acceptable salt thereof, or combinations thereof.

In one aspect, a method is provided for releasing a macromolecule, e.g., an oligonucleotide and/or a polypeptide, from an endosome. In one aspect, the method includes: contacting the cell with the macromolecule, e.g., oligonucleotide and/or polypeptide, wherein the macromolecule, e.g., oligonucleotide and/or polypeptide, is internalized by the cell through endocytosis and encapsulated within the endosome; and contacting the cell with a compound described herein, wherein the compound facilitates release of the macromolecule, e.g., oligonucleotide and/or polypeptide, from the endosome. In some embodiments, the oligonucleotide is an ASO. In some embodiments, the oligonucleotide is an SSO. In some embodiments, the oligonucleotide is a siRNA. In some embodiments, the oligonucleotide is a guide RNA. In some embodiments, the polypeptide is a Cas protein. In some embodiments, the polypeptide is a recombinase. In some embodiments, the polypeptide is Cre. In some embodiments, the polypeptide is FLP. In some embodiments, the polypeptide is a Cas protein, and the polynucleotide is a guide RNA. In some embodiments, the compound has a structure represented by Formula I, Formula Ia, Formula Ib, or Formula Ic, or is a compound listed in Table 1, or a pharmaceutically acceptable salt thereof, or combinations thereof. In some embodiments, the compound is any one of Compounds 4, 8, and 32, a pharmaceutically acceptable salt thereof, or combinations thereof.

In one aspect, a method is provided for the treatment and/or prevention of a disorder in a subject, such as a genetic disorder. In one aspect, the method includes administering to the subject a therapeutically effective amount of a macromolecule, e.g., an oligonucleotide and/or polypeptide, and an effective amount of a compound described herein. In one aspect, the compound is administered to the subject concurrently with the macromolecule, e.g., oligonucleotide and/or polypeptide. In one aspect, the compound is administered to the subject after administration of the macromolecule, e.g., oligonucleotide and/or polypeptide. In one aspect, the compound is administered to the subject up to about 48 hours after administration of the macromolecule, e.g., oligonucleotide and/or polypeptide. In one aspect, the compound is administered to the subject about 12 hours to about 48 hours after administration of the macromolecule, e.g., oligonucleotide and/or polypeptide. In one aspect, the compound is administered to the subject at least about 12 hours or about 24 hours and up to about 36 hours or about 48 hours after administration of the macromolecule, e.g., oligonucleotide and/or polypeptide. In one aspect, administration includes parenteral administration. In one aspect, parenteral administration includes intravenous (IV) or subcutaneous (SC) administration. In one aspect, the subject is a mammal. In one aspect, the subject is a human. In some embodiments, the oligonucleotide is an ASO. In some embodiments, the oligonucleotide is an SSO. In some embodiments, the oligonucleotide is a siRNA. In some embodiments, the oligonucleotide is a guide RNA. In some embodiments, the polypeptide is a Cas protein. In some embodiments, the polypeptide is a recombinase. In some embodiments, the polypeptide is Cre. In some embodiments, the polypeptide is FLP. In some embodiments, the polypeptide is a Cas protein, and the polynucleotide is a guide RNA. In some embodiments, the compound has a structure represented by Formula I, Formula Ia, Formula Ib, or Formula Ic, or is a compound listed in Table 1, or a pharmaceutically acceptable salt thereof, or combinations thereof. In some embodiments, the compound is any one of Compounds 4, 8, and 32, a pharmaceutically acceptable salt thereof, or combinations thereof.

In one aspect, a method is provided for the treatment and/or prevention of a disorder in a subject. In one aspect, the method includes: isolating a cell from the subject; contacting the isolated cell with a therapeutically effective amount of a macromolecule, e.g., an oligonucleotide and/or polypeptide, and an effective amount of a compound described herein to produce an engineered cell; and transplanting the engineered cell in the subject. In one aspect, the isolated cell is contacted with a composition comprising about 0.025 μM to about M of the macromolecule, e.g., oligonucleotide and/or polypeptide. In one aspect, the isolated cell is contacted with about 0.1 μM to about 10 μM, about 0.1 μM to about 5 μM, or about 0.1 μM to about 1 μM of the macromolecule, e.g., oligonucleotide and/or polypeptide. In one aspect, the isolated cell is contacted with a composition that includes about 1 μM to about 20 μM of a compound described herein. In one aspect, the isolated cell is contacted with a composition that includes about 1 μM to about 10 μM of a compound described herein. In one aspect, the isolated cell is contacted with a composition that includes at least about 1 μM, about 1.5 μM, about 2 μM, about 2.5 μM, about 3 μM, about 3.5 μM, about 4 μM, about 4.5 μM, or about 5 μM and up to about 6 μM, about 6.5 μM, about 7 μM, about 7.5 μM, about 8 μM, about 8.5 μM, about 9 μM, about 9.5 μM, about 10 μM, about 10.5 μM, about 11 μM, about 11.5 μM, about 12 μM, about 12.5 μM, about 13 μM, about 13.5 μM, about 14 μM, about 14.5 μM, about 15 μM, about 15.5 μM, about 16 μM, about 16.5 μM, about 17 μM, about 17.5 μM, about 18 μM, about 18.5 μM, about 19 μM, about 19.5 μM, or about 20 μM of a compound described herein. In some embodiments, the oligonucleotide is an ASO. In some embodiments, the oligonucleotide is an SSO. In some embodiments, the oligonucleotide is a siRNA. In some embodiments, the oligonucleotide is a guide RNA. In some embodiments, the polypeptide is a Cas protein. In some embodiments, the polypeptide is a recombinase. In some embodiments, the polypeptide is Cre. In some embodiments, the polypeptide is FLP. In some embodiments, the polypeptide is a Cas protein, and the polynucleotide is a guide RNA. In some embodiments, the compound has a structure represented by Formula I, Formula Ia, Formula Ib, or Formula Ic, or is a compound listed in Table 1, or a pharmaceutically acceptable salt thereof, or combinations thereof. In some embodiments, the compound is any one of Compounds 4, 8, and 32, a pharmaceutically acceptable salt thereof, or combinations thereof.

In one aspect, the isolated cell is contacted with the macromolecule, e.g., oligonucleotide and/or polypeptide, and the compound at approximately the same time. In one aspect, the isolated cell is contacted with a composition that includes the macromolecule, e.g., oligonucleotide and/or polypeptide, and the compound. In one aspect, the isolated cell is contacted with the macromolecule, e.g., oligonucleotide and/or polypeptide, before the isolated cell is contacted with the compound. In one aspect, the isolated cell is contacted with the macromolecule, e.g., oligonucleotide and/or polypeptide, up to about 48 hours before the isolated cell is contacted with the compound. In one aspect, the isolated cell is contacted with the macromolecule, e.g., oligonucleotide and/or polypeptide, about 12 hours to about 48 hours before the isolated cell is contacted with the compound. In one aspect, the isolated cell is contacted with the macromolecule, e.g., oligonucleotide and/or polypeptide, at least about 12 hours or about 24 hours and up to about 36 hours or about 48 hours before the isolated cell is contacted with the compound. In one aspect, the subject is a mammal. In one aspect, the subject is a human. In some embodiments, the oligonucleotide is an ASO. In some embodiments, the oligonucleotide is an SSO. In some embodiments, the oligonucleotide is a siRNA. In some embodiments, the oligonucleotide is a guide RNA. In some embodiments, the polypeptide is a Cas protein. In some embodiments, the polypeptide is a recombinase. In some embodiments, the polypeptide is Cre. In some embodiments, the polypeptide is FLP. In some embodiments, the polypeptide is a Cas protein, and the polynucleotide is a guide RNA. In some embodiments, the compound has a structure represented by Formula I, Formula Ia, Formula Ib, or Formula Ic, or is a compound listed in Table 1, or a pharmaceutically acceptable salt thereof, or combinations thereof. In some embodiments, the compound is any one of Compounds 4, 8, and 32, a pharmaceutically acceptable salt thereof, or combinations thereof.

Provided herein is a use of a compound described herein in the manufacture of a medicament for gene therapy. Provided herein is a use of a compound of Formula I, or a pharmaceutically acceptable salt thereof, in the manufacture of a medicament for gene therapy. Provided herein is a use of a compound described herein in the manufacture of a medicament for gene therapy. Provided herein is a use of a compound of Formula Ia, or a pharmaceutically acceptable salt thereof, in the manufacture of a medicament for gene therapy. Provided herein is a use of a compound of Formula Ib, or a pharmaceutically acceptable salt thereof, in the manufacture of a medicament for gene therapy. Provided herein is a use of a compound of Formula Ic, or a pharmaceutically acceptable salt thereof, in the manufacture of a medicament for gene therapy. Provided herein is a use of a compound of Formula I, Formula Ia, Formula Ib, or Formula Ic, or the compounds listed in Table 1, or a pharmaceutically acceptable salt thereof, or a combination thereof, in the manufacture of a medicament for gene therapy. Provided herein is a use of any one of Compounds 4, 8, and 32, a pharmaceutically acceptable salt thereof, or a combination thereof, in the manufacture of a medicament for gene therapy.

Provided herein is a use of a compound described herein and a macromolecule, e.g., an oligonucleotide and/or polypeptide, in the manufacture of a medicament for gene therapy. Provided herein is a use a compound of Formula I, or a pharmaceutically acceptable salt thereof, and a macromolecule, e.g., an oligonucleotide and/or polypeptide, in the manufacture of a medicament for gene therapy. Provided herein is a use a compound of Formula Ia, or a pharmaceutically acceptable salt thereof, and a macromolecule, e.g., an oligonucleotide and/or polypeptide, in the manufacture of a medicament for gene therapy. Provided herein is a use a compound of Formula Ib or a pharmaceutically acceptable salt thereof, and a macromolecule, e.g., an oligonucleotide and/or polypeptide, in the manufacture of a medicament for gene therapy. Provided herein is a use a compound of Formula Ic or a pharmaceutically acceptable salt thereof, and a macromolecule, e.g., an oligonucleotide and/or polypeptide, in the manufacture of a medicament for gene therapy. Provided herein is a use of a compound of Formula I, Formula Ia, Formula Ib, Formula Ic, or the compounds listed in Table 1, or a pharmaceutically acceptable salt thereof, or a combination thereof, and a macromolecule, e.g., an oligonucleotide and/or polypeptide, in the manufacture of a medicament for gene therapy. Provided herein is a use of any one of Compounds 4, 8, and 32, a pharmaceutically acceptable salt thereof, or a combination thereof, and a macromolecule, e.g., an oligonucleotide and/or polypeptide, in the manufacture of a medicament for gene therapy.

Provided herein is a kit that includes a compound described herein and a macromolecule, e.g., an oligonucleotide and/or polypeptide. In one aspect, the kit includes a compound of Formula I, or a pharmaceutically acceptable salt thereof, and a macromolecule, e.g., an oligonucleotide and/or polypeptide. In one aspect, the kit includes a compound of Formula Ia, or a pharmaceutically acceptable salt thereof, and a macromolecule, e.g., an oligonucleotide and/or polypeptide. In one aspect, the kit includes a compound of Formula Ib, or a pharmaceutically acceptable salt thereof, and a macromolecule, e.g., an oligonucleotide and/or polypeptide. In one aspect, the kit includes a compound of Formula Ic, or a pharmaceutically acceptable salt thereof, and a macromolecule, e.g., an oligonucleotide and/or polypeptide. In one aspect, the kit includes a compound of Formula I, Formula Ia, Formula Ib, or Formula Ic, or the compounds listed in Table 1, or a pharmaceutically acceptable salt thereof, or a combination thereof, and a macromolecule, e.g., an oligonucleotide and/or polypeptide. In one aspect, the kit includes any one of Compounds 4, 8, and 32, or a pharmaceutically acceptable salt thereof, or a combination thereof, and a macromolecule, e.g., an oligonucleotide and/or polypeptide.

The entire contents of all publications, patents, and patent applications referenced herein are hereby incorporated herein by reference.

The specific examples included herein are for illustrative purposes only and are not to be considered as limiting to this disclosure. Moreover, the compositions and methods provided herein have been described in relation to certain embodiments thereof, and many details have been set forth for purposes of illustration. It will be apparent to those skilled in the art that the disclosure is susceptible to additional embodiments and that certain details described herein may be varied without departing from the basic principles of the disclosure.

### WORKING EXAMPLES

### Example 1. Preparation of Splice-Switching Oligonucleotides (SSOs)

An 18 nt splice switching oligonucleotide that binds to Luc705 (5′-CCUCUUACCUCAGUUACA-3′ SEQ ID NO:1) (705-SSO) was synthesized with 2′-O-methylated (2′-OMe)-modified bases and phosphorothioate (PS)-saturated backbone linkages. Luc705-SSO labeled with Alexa Fluor™ 488 (green) fluorescent labels were synthesized as shown in Table 3. For the non-fluorescent oligos, HPLC purification and a Na+ salt exchange step were included in the synthesis. SSOs were shipped in IDTE buffer (nuclease-free H₂O supplemented with 10 mM Tris and 0.1 mM EDTA) at a pH of 8.0 at 100 μM.

TABLE 3
Nomenclature, Sequence, Chemical
Modifications, and Size of SSOs

| | | Size |
|---|---|---|
| Name | Sequence | (nt) |
| Luc705- | 5′-mC*mC*mU*mC*mU*mU*mA*mC*mC* | 18 |
| SSO | mU*mC*mA*mG*mU*mU*mA*mC*mA-3′ | |
| A488- | 5′-Alexa488- | 18 |
| SSO | mC*mC*mU*mC*mU*mU*mA*mC*mC*mU* | |
| | mC*mA*mG*mU*mU*mA*mC*mA-3′ | |

*, PS linkage; m, 2-OMe nucleotide

### Example 2. Preparation of Small Molecule Compound (SMC) Library

Small-molecule compounds (SMC) were screened for their ability to increase the activity of a gapmer antisense oligonucleotide (ASO) by co-treatment of cells with the SMC and ASO as shown in the subsequent examples. Representative procedures for synthesizing the SMCs are provided below.

### General Procedure for Preparation of Compounds 2-7 and 19-31

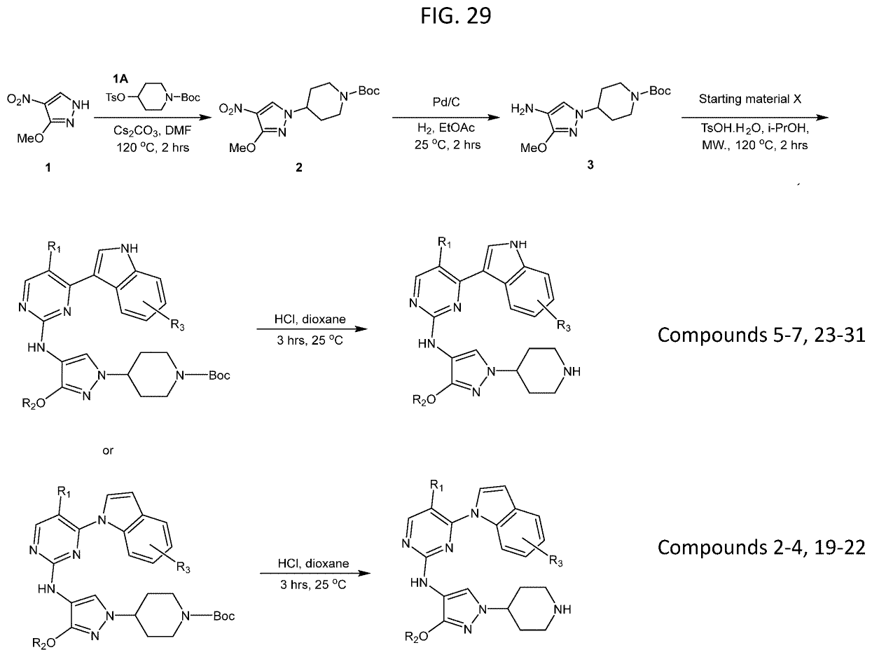

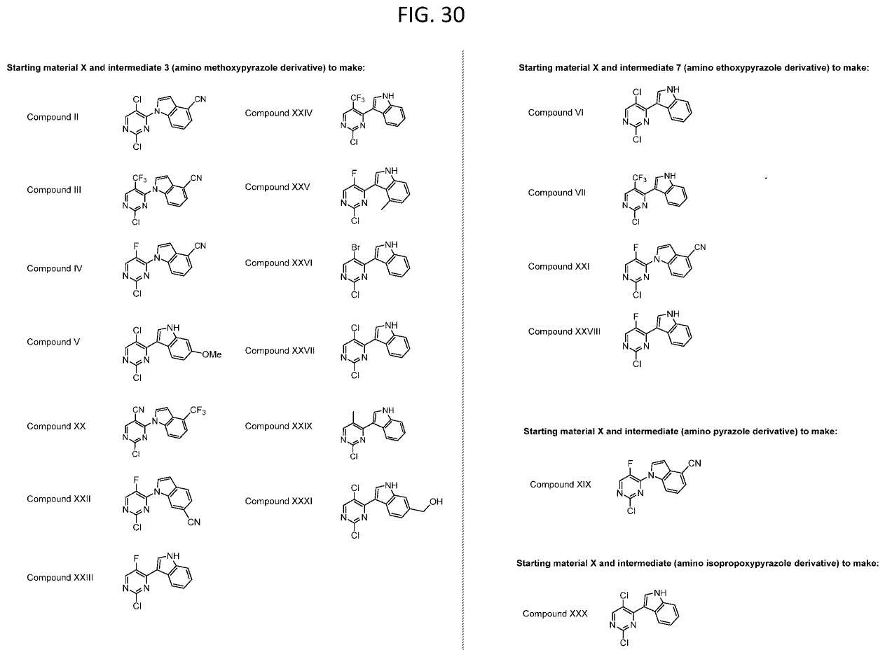

The general procedure for the preparation of Compounds 2-7 and 19-31 is shown schematically in FIG. 29-30 where FIG. 29 provides the overall generic scheme and includes starting material X shown in step 3. FIG. 30 describes the definition of starting material X and required intermediate for each of the depicted compounds (e.g., Compound II depicted in FIG. 30 is the starting material “X” in the preparation described in FIG. 29 for Compound 2). Further descriptions of exemplified intermediates and Compounds are provided below. The amino pyrazole and amino isopropoxypyrazole derivative intermediates identified in FIG. 30 that are used to make Compounds 19 and 30, respectively, were prepared according to the same procedures described below for intermediate 3 (amino methoxypyrazole) and 7 (amino ethoxypyrazole), respectively.

### General Procedure for Preparation of Intermediate 2

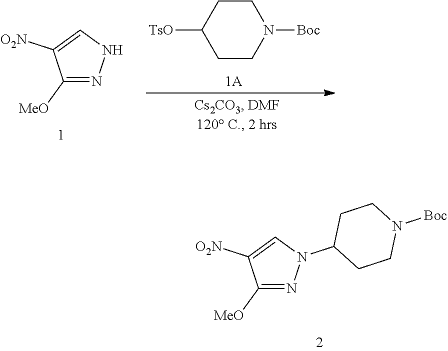 [SMILES: COc1n[nH]cc1[N+](=O)[O-] ; Cc1ccc(S(=O)(=O)OC2CCN(C(=O)OC(C)(C)C)CC2)cc1 ; COc1nn(C2CCN(C(=O)OC(C)(C)C)CC2)cc1[N+](=O)[O-]]

1. Add Cpd.1 (9.00 g, 62.8 mmol, 1.00 eq) and Cpd.1A (28.8 g, 81.1 mmol, 1.29 eq) into a flask charged with DMF (63 mL).
2. Add Cs₂CO₃ (30.5 g, 93.7 mmol, 1.49 eq) to the mixtures.
3. Degas with N₂ for 3 times.
4. Stir at 120° C. for 2 hrs.
5. TLC show Cpd.1 consumed, a new spot formed.
6. Adjust the PH of the mixture to 7-8 with HCl (1 M).
7. Extract with mixture with DCM (200 mL×3).
8. Wash the organic layer with brine (100 mL×2) and dried over magnesium sulfate.
9. Concentrate the organic layer in vacuo to remove DCM.
10. The residue was purified by column chromatography (SiO₂, Petroleum ether/Ethyl acetate=30/1 to 3/1).
11. Obtain Cpd.2 (18.1 g, 70.5% yield, 80.0% purity) as yellow solid.

¹H NMR: 400 MHz DMSO-d₆ δ 8.746 (s, 1H), 7.94 (s, 1H), 4.69-4.68 (m, 1H), 4.31-4.26 (m, 2H), 4.05 (s, 3H), 3.33 (s, 4H), 2.88 (s, 3H), 2.72 (s, 1H), 2.01-1.98 (m, 2H), 1.81-1.74 (m, 2H), 1.40 (s, 9H)

### General Procedure for Preparation of Intermediate 3

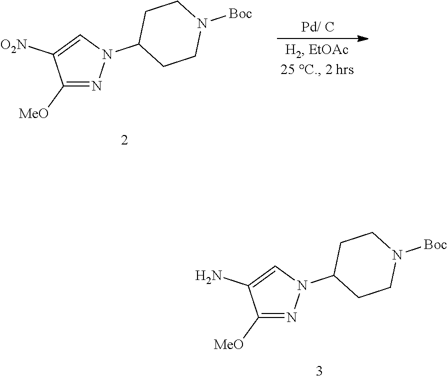 [SMILES: COc1nn(C2CCN(C(=O)OC(C)(C)C)CC2)cc1[N+](=O)[O-] ; COc1nn(C2CCN(C(=O)OC(C)(C)C)CC2)cc1N]

1. Add Cpd.2 (17.0 g, 52.0 mmol, 1.00 eq) in a flask charged with EtOAc (110 mL).
2. Add Pd/C (1.70 g, 52.0 mmol, 10% purity, 1.00 eq) to the mixture.
3. Add H₂ (15 Psi) to the mixture.
4. Stir at 25° C. for 10 hrs.
5. TLC (Petroleum ether/Ethyl acetate=3/1, product Rf=0.42) shows Cpd.3 formed.
6. Filtered through celites.
7. Wash filter cake with EtOAc(150 mL×3).
8. Concentrate the filtrate in vacuo to remove EtOAc.
9. The residue was purified by column chromatography (SiO₂, Petroleum ether/Ethyl acetate=30/1 to 3/1).
10. Obtain Cpd.3 (10.9 g, 70.6% yield) as black-blue oil.

¹H NMR: 400 MHz DMSO-d₆ δ 7.00 (s, 1H), 4.01-3.94 (m, 3H), 3.52 (s, 3H), 2.88-2.73 (m, 2H), 1.88-1.84 (m, 2H), 1.66-1.60 (m, 2H), 1.40 (s, 9H)

### Alternate Method for Preparation of Intermediate Compound 3

A solution of compound 2 and iron(iii)chloride (3.41 g, 1.05 mmol) in MeOH) (21.14 ml) was treated with hydrazine (2.197 ml, 70.00 mmol) and brought to reflux overnight. Reaction was filtered. Contraction of filtrate afforded residue. The residue was redissolved in DCM/MeOH (10:1) and washed with water. Concentration of organic layer yielded tert0butyl 4-(4-amino-3methoxy-1H-pyrazol-1-yl)piperidine-1-carboxylate (2.000 g, 96%).

### General Procedure for Preparation of Intermediate 4-1

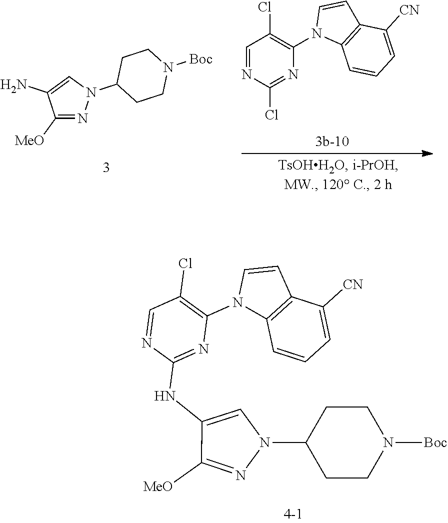 [SMILES: COc1nn(C2CCN(C(=O)OC(C)(C)C)CC2)cc1N ; N#Cc1cccc2c1ccn2-c1nc(Cl)ncc1Cl ; COc1nn(C2CCN(C(=O)OC(C)(C)C)CC2)cc1Nc1ncc(Cl)c(-n2ccc3c(C#N)cccc32)n1]

1. Add Cpd.3 (0.50 g, 1.69 mmol, 1.00 eq) and Cpd.3b-10 (444 mg, 1.54 mmol, 0.91 eq) charged with i-PrOH (10.0 mL) to a microwave vial.
2. Add TsOH (529 mg, 3.08 mmol, 1.82 eq) to the mixture.
3. Stir at 120° C. for 2 hrs.
4. LCMS (ET37912-40-Pla) shows Cpd.4-1 formed.
5. Adjust the pH Of the mixture to 7-8 with Sat. NaHCO₃.
6. Extract the mixture with EtOAc (50 mL×3).
7. Wash the organic layer with brine (30 mL×2).
8. Concentrate in vacuo to remove EtOAc.
9. Obtain Cpd.4-1 (0.40 g, crude) as yellow solid.

### Example Procedure for Preparation of Compound 2

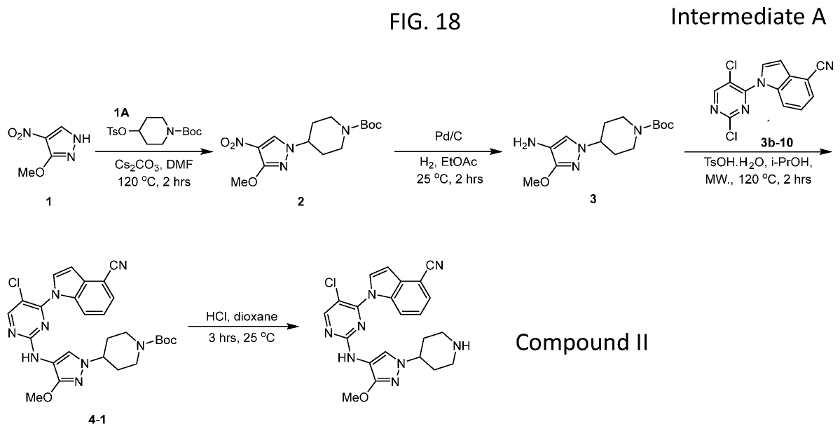

The procedure for the preparation of Compound 2 is shown schematically in FIG. 18.
1. Dissolve Cpd.4-1 (0.90 g, 1.64 mmol, 1.00 eq) in HCl/dioxane (4 M, 9.00 mL, 21.9 eq).
2. Stir for 3 h at 25° C.
3. LCMS (product Rt=0.582 min) shows Compound 2 formed.
4. Add sat. NaHCO₃ (50.0 mL) to the solution, pH=8.
5. Extract with EtOAc (100.0 mL×2), separate the organic layer and dry over Na₂SO₄, concentrate in vacuum.
6. Purity by prep-HPLC(column: Phenomenex Gemini-NX 80*40 mm*3 um; mobile phase: [water(10 mM NH4HCO₃)-ACN]; B %: 20%-40%, 8 min).
7. Obtain Compound 2 (0.062 g, 8.34% yield) as white solid.

¹H NMR: 400 MHz DMSO-d₆ δ 8.44 (s, 1H), 8.08 (s, 1H), 7.85 (d, J=8.0 Hz, 1H), 7.73 (s, 1H), 7.61 (d, J=8.0 Hz, 1H), 7.34 (t, J=8.0 Hz, 1H), 3.90 (s, 1H), 3.77 (m, 3H), 2.96 (s, 2H), 1.84 (s, 2H), 1.67-1.60 (m, 2H).

### Example Procedure for Preparation of Compound 6

The procedure for the preparation of Compound 6 is the same as described above for Compound 2 but starting from intermediate 1, where the methoxy substitutent is replaced with an ethoxy substituent and a modified Cpd.3b-10 (i.e. indole intermediate) lacking the cyano substituent.

General Procedure for Preparation of Intermediate 3b-10

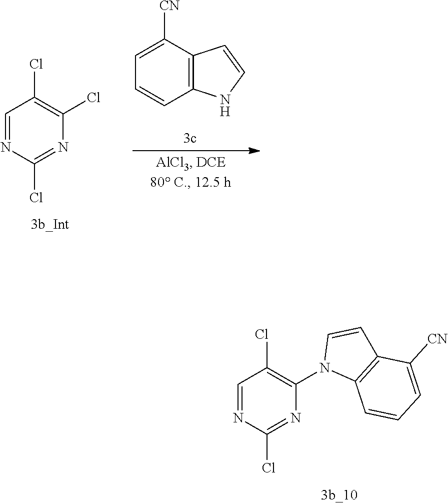

1. Add Cpd.3C (4.84 g, 26.3 mmol, 1.50 eq) charged with DCE (100 mL) into a flask.
2. Add AlCl₃ (3.52 g, 26.3 mmol, 1.44 mL, 1.50 eq) to the mixture, the color becomes yellow.
3. Stir at 80° C. for 0.5 hrs.
4. Add Cpd.3b_Int (2.5 g, 17.59 mmol, 1.00 eq) to the mixture, the color becomes orange red.
5. Stir at 80° C. for 12 hrs.
6. LCMS show Cpd.3b_10 was formed.
7. Add the mixture to ice-water mixture (200 mL).
8. Extract the mixture with EtOAc (250 mL×3).
9. Wash the organic layer with brine (100 mL×2).
10. Concentrate in vacuo to remove EtOAc.
11. The residue was purified by column chromatography (SiO₂, Petroleum ether/Ethyl acetate=30/1 to 1/1).
12. Obtain Cpd.3b_10 (0.50 g, 9.83% yield) as orange solid.

¹H NMR: 400 MHz DMSO-d₆ δ 8.95 (s, 1H), 8.37 (s, 1H), 7.91 (d, J=8.0 Hz, 1H), 7.69 (d, J=8.0 Hz, 1H), 7.41 (t, J=8 Hz, 1H).

### General Procedure for Preparation of Intermediate 4-2

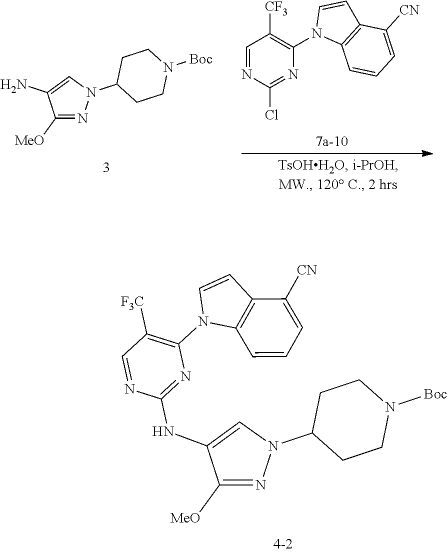 [SMILES: COc1nn(C2CCN(C(=O)OC(C)(C)C)CC2)cc1N ; N#Cc1cccc2c1ccn2-c1nc(Cl)ncc1C(F)(F)F ; COc1nn(C2CCN(C(=O)OC(C)(C)C)CC2)cc1Nc1ncc(C(F)(F)F)c(-n2ccc3c(C#N)cccc32)n1]

1. Add Cpd.3 (0.50 g, 1.69 mmol, 1.00 eq) and Cpd.7a-10 (545 mg, 1.69 mmol, 1.00 eq) charged with i-PrOH (10.0 mL) to a microwave vial.
2. Add TsOH (529.65 mg, 3.08 mmol, 1.82 eq) to the mixture.
3. Stir at 120° C. for 2 hrs.
4. LCMS shows Cpd.4-2 formed.
5. Adjust the pH of the mixture to 7-8 with Sat. NaHCO₃.
6. Extract the mixture with EtOAc (30 mL×3).
7. Wash the organic layer with brine (15 mL×2).
8. Concentrate in vacuo to remove EtOAc.
9. Obtain Cpd.4-2 (0.40 g, crude) as yellow solid.

### General Procedure for Preparation of Compound 3

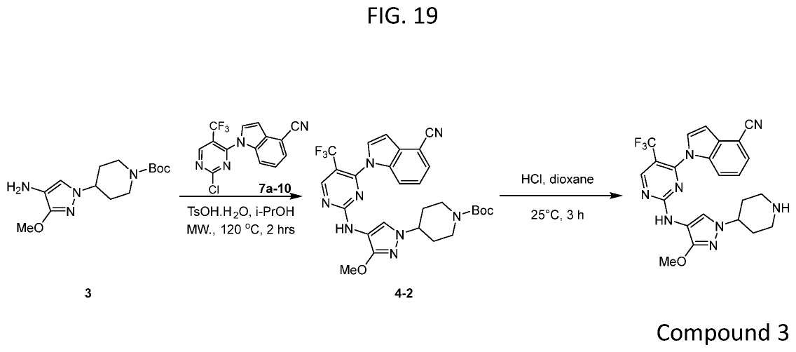

The procedure for the preparation of Compound 3 is shown schematically in FIG. 19.

Dissolve Cpd.4-2 (0.90 g, 1.54 mmol, 1.00 eq) in HCl/dioxane (4 M, 9.00 mL, 23.3 eq).
1. Stir for 3 h at 25° C.
2. LCMS (product Rt=0.602 min) shows Compound 3 formed.
3. Add sat. NaHCO₃ (10.0 mL) to the solution, pH=8.
4. Extract with EtOAc (50.0 mL×2), separate the organic layer and dry over Na₂SO₄, concentrate in vacuum.
5. Purity by prep-HPLC (column: Phenomenex Gemini-NX 80*40 mm*3 um; mobile phase: [water (10 mM NH4HCO3)-ACN]; B %: 15%-35%, 8 min).
6. Obtain Compound 3 (0.08 g, 10.7% yield, 100% purity) as yellow solid.

¹H NMR: 400 MHz DMSO-d₆ δ 8.84 (brs, 1H), 7.84 (d, J=8.0 Hz, 1H), 7.72 (s, 1H), 7.56 (d, J=7.2 Hz), 7.33 (t, J=8 Hz, 1H), 3.91 (brs, 1H), 3.90 (s, 3H), 2.99 (s, 2H), 2.53-2.50 (m, 1H), 1.85 (s, 1H), 1.62 (brs, 1H).

General Procedure for Preparation of Intermediate 7a-10

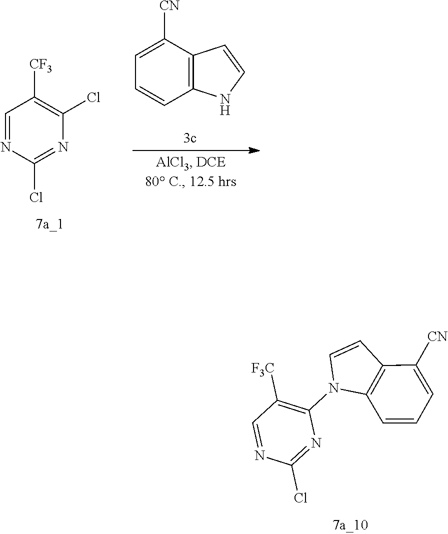

1. Add Cpd.7a_1 (5.72 g, 26.3 mmol, 1.50 eq) charged with DCE (100 mL) into a flask.
2. Add AlCl₃ (2.34 g, 17.5 mmol, 961 μL, 1.00 eq) to the mixture, the color becomes yellow.
3. Stir at 80° C. for 0.5 hrs.
4. Add Cpd.3C (2.50 g, 17.5 mmol, 1.00 eq) to the mixture, the color becomes orange red.
5. Stir at 80° C. for 12 hrs.
6. LCMS shows Cpd.7a_10 is formed.
7. Add the mixture to ice-water mixture (200 mL).
8. Extract the mixture with EtOAc (200 mL×3).
9. Wash the organic layer with brine (100 mL×2).
10. Concentrate in vacuo to remove EtOAc.
11. The residue was purified by column chromatography (SiO₂, Petroleum ether/Ethyl acetate=20/1 to 1/1)
12. Obtain Cpd.7a_10 (1.60 g, 28.2% yield) as yellow solid

¹H NMR: 400 MHz DMSO-d₆ δ 9.26 (s, 1H), 8.03 (s, 1H), 7.91 (d, J=8.0 Hz, 1H), 7.73 (d, J=8.0 Hz, 1H), 7.41 (t, J=8 Hz, 1H).

### General Procedure for Preparation of Intermediate 4-3

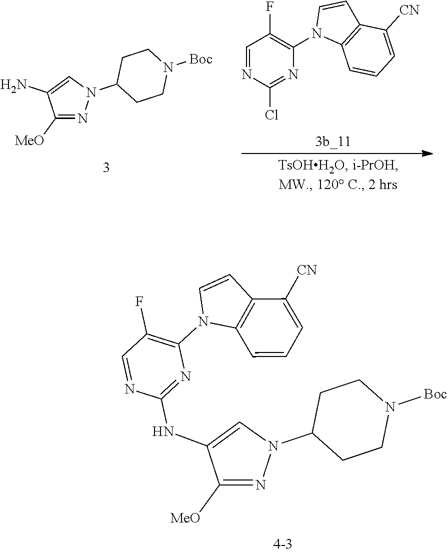 [SMILES: COc1nn(C2CCN(C(=O)OC(C)(C)C)CC2)cc1N ; N#Cc1cccc2c1ccn2-c1nc(Cl)ncc1F ; COc1nn(C2CCN(C(=O)OC(C)(C)C)CC2)cc1Nc1ncc(F)c(-n2ccc3c(C#N)cccc32)n1]

1. Add tert-butyl Cpd.3 (399 mg, 1.47 mmol, 0.91 eq) charged with i-PrOH (7.00 mL) to a microwave vial.
2. Add TsOH (504 mg, 2.93 mmol, 1.82 eq) to the mixture.
3. Stir at 120° C. for 2 hrs.
4. LCMS (product Rt=0.857 min) shows Cpd.4-3. formed.
5. Adjust the PH Of the mixture to 7-8 with Sat. NaHCO₃.
6. Extract the mixture with EtOAc (100 mL×3).
7. Wash the organic layer with brine (100 mL×2).
8. Concentrate the organic in vacuo to remove EtOAc.
9. Obtain Cpd.4-3 (0.6 g, crude) as white solid.

### General Procedure for Preparation of Compound 4 and Compounds 32-35

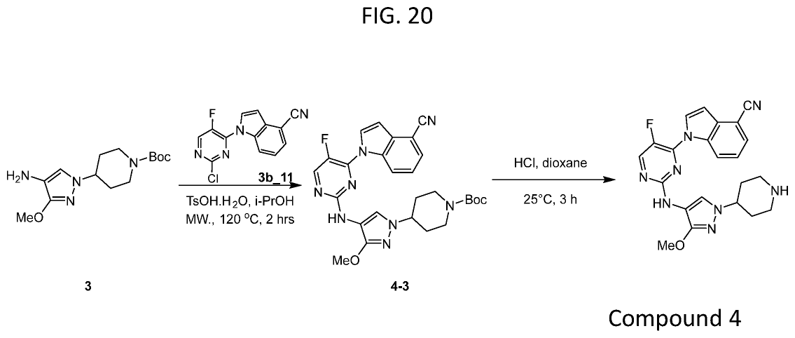

The procedure for the preparation of Compound 4 and Compounds 32-35 is shown schematically in FIG. 20, using Compound 4 as an example.
1. Dissolve Cpd.4-3 (0.60 g, 1.13 mmol, 1.00 eq) in HCl/dioxane (4 M, 4.20 mL, 14.9 eq).
2. Stir at 25° C. for 2 hrs.
3. LCMS (product Rt=0.348 min) shows Compound 4 formed.
4. Add sat. NaHCO₃ (50.0 mL) to the solution, pH=8.
5. Extract with EtOAC (100.0 mL×2), separate the organic layer and dry over Na₂SO₄. concentrate in vacuum.
6. Purity by prep-HPLC (column: Phenomenex Gemini-NX 80*40 mm*3 um; mobile phase: [water (10 mM NH₄HCO₃)-ACN]; B %: 20%-50%, 8 min).

Obtain Compound 4 (0.05 g, 10.0% yield, 98.3% purity) as yellow solid. Note that Compounds 32-35 follow the same procedure as described above for Compound 4, but replacing intermediate 1A (in the preparation of Intermediate 2→Intermediate 3→Intermediate 4-3) with the following compounds:

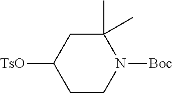 [SMILES: Cc1ccc(S(=O)(=O)OC2CCN(C(=O)OC(C)(C)C)C(C)(C)C2)cc1]

(to result in Compound 32, 0.03 g, 4% yield, 96.5% purity as yellow solid);

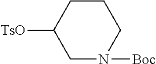 [SMILES: Cc1ccc(S(=O)(=O)OC2CCCN(C(=O)OC(C)(C)C)C2)cc1]

(to result in Compound 33, 0.05 g, 5% yield, 99.6% purity as yellow solid);

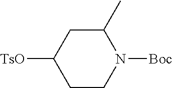 [SMILES: Cc1ccc(S(=O)(=O)OC2CCN(C(=O)OC(C)(C)C)C(C)C2)cc1]

(to result in Compound 34, 0.03 g, 2% yield, 97% purity as yellow solid); and

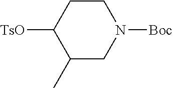 [SMILES: Cc1ccc(S(=O)(=O)OC2CCN(C(=O)OC(C)(C)C)CC2C)cc1]

(to result in Compound 35, 0.03 g, 3% yield, 98.5% purity as yellow solid)

Compounds 32-35 can be further separated into their isomeric parts by supercritical fluid chromatography (SFC). For example, Compound 32 was separated into its enantiomers by SFC with use of a mobile phase consisting of ethanol/DEA/CO2 and a YMC SA (IA) column (particle size 5 um). The preparative conditions consisted of a flow of 3.5 ml/min at 40 deg C. and the compounds were detected at 272 nm. The enantiomeric excess of both enantiomers exceeded 99% ee according to LC/MS analysis. The fractions containing the separated enantiomers were evaporated which yielded the final compounds as solids.

¹H NMR: 400 MHz DMSO-d₆

Compound 4: δ 8.81 (brs, 1H), 8.60 (d, J=4.0 Hz, 1H), 8.08 (s, 1H), 7.73 (s, 1H), 7.32 (brs, 1H), 6.95 (d, J=4.0 Hz, 1H), 3.99-3.94 (m, 1H), 3.79 (s, 1H), 3.01 (d, J=8.0 Hz, 1H), 2.57-2.54 (m, 2H), 1.90-1.87 (m, 2H), 1.71-1.68 (m, 2H).

Compound 32: δ 8.82 (br s, 1H), 8.77 (s, 1H), 8.09 (s, 1H), 7.74 (m, 2H), 7.34 (br s, 1H), 6.96 (s, 1H), 4.17 (m, 1H), 3.79 (s, 3H), 2.80 (m, 2H), 1.75-1.87 (dd, 2H), 1.51-1.61 (m, 3H), 1.10 (s, 3H), 1.06 (s, 3H).

Compound 33: δ 8.81 (br s, 1H), 8.60 (s, 1H), 8.09 (s, 1H), 7.75 (m, 2H), 7.33 (br s, 1H), 6.96 (s, 1H), 3.90 (m, 1H), 3.79 (s, 3H), 3.12 (d, 1H), 2.84 (d, 1H), 2.65 (t, 1H), 2.44 (t, 1H), 2.01 (m, 1H), 1.70-1.81 (m, 2H), 1.45 (m, 1H).

Compound 34: δ 8.56 (m, 3H), 8.07 (m, 1H), 7.70 (m, 2H), 7.35 (t, 1H), 6.94 (m, 1H), 3.99 (m, 1H), 3.82 (s, 3H), 3.05 (m, 2H), 2.66 (m, 2H), 1.93 (t, 2H), 1.68 (m, 1H), 1.36 (m, 1H), 1.05 (d, 3H).

Compound 35: δ 8.55 (br s, 3H), 8.05 (s, 1H), 7.64-7.71 (m, 2H), 7.33 (m, 1H), 6.93 (br s, 1H), 4.16 (br s, 1H), 3.83 (br s, 3H), 3.15 (br s, 1H), 2.80 (m, 2H), 2.65 (m, 1H), 2.21 (br s, 1H), 1.97 (m, 1H), 1.74 (m, 1H), 0.71 (d, 3H).

### General Procedure for Preparation of Compounds 8-17

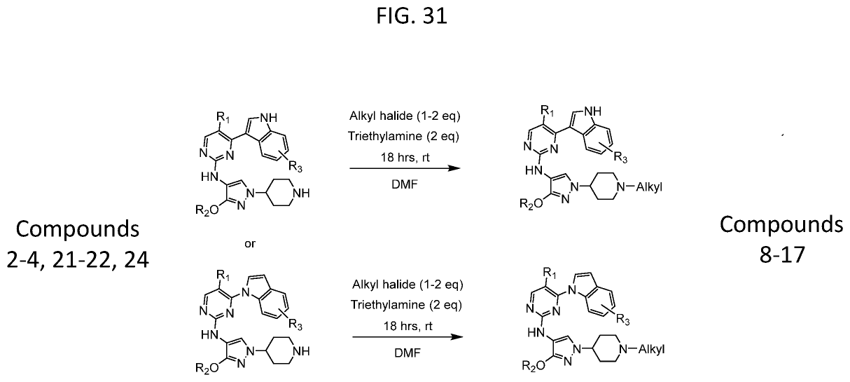

The procedure for the preparation of Compounds 8-17 follows the procedure above for Compound 4 plus an additional alkylation step as shown in FIG. 31:
1. Add small molecule (i.e., either Compound 2-4, 21-22 or 24) with free NH (1.00 eq) to a flask charged with DMF (3 mL).
2. Add triethylamine (2.0 eq) to the mixture.
3. Add alkyl bromide or alkyl iodide (1.00 to 2.00 eq) to the mixture.
4. Stir at 20° C. for 18 hours.
5. LCMS shows starting material consumed and the reaction completed.
6. Add aqueous ammonia 30% (three drops) to quench excess alkyl halide.
7. Concentrate reaction mixture in vacuo to remove DMF.
8. The residue was purified by column chromatography (SiO2, Heptane/Ethyl acetate=100/0 to 0/100 followed by DCM/MeOH=100/0 to 60/40).
9. Obtain Compounds 8-17 as yellow solid.

¹H NMR: 500 MHz DMF-d7

Compound 8: δ 1.94-2.1 (m, 6H), 2.24 (s, 3H), 2.89 (d, J=10.7 Hz, 2H), 3.89 (s, 3H), 3.96 (dd, J=11.1, 5.4 Hz, 1H), 7.02 (d, J=3.6 Hz, 1H), 7.44 (s, 1H), 7.79 (d, J=7.4 Hz, 1H), 7.89 (s, 1H), 8.18 (dd, J=3.6, 2.2 Hz, 1H), 8.63 (d, J=4.3 Hz, 1H).

Compound 9: δ 1.10 (s, 6H), 1.95-2.5 (m, 6H), 3.06 (bs, 2H), 3.89 (s, 3H), 4.03 (m, 1H), 7.03 (d, J=3.6 Hz, 1H), 7.45 (t, J=7.7 Hz, 1H), 7.78 (d, J=7.4 Hz, 1H), 7.90 (s, 1H), 8.16-8.21 (m, 1H), 8.64 (d, J=4.3 Hz, 1H).

Compound 10: δ 0.91 (t, J=7.4 Hz, 3H), 1.52 (s, 2H), 2.04 (s, 6H), 2.32 (s, 2H), 2.99 (s, 2H), 3.89 (s, 3H), 4.01 (s, 1H), 7.03 (d, J=3.6 Hz, 1H), 7.45 (s, 1H), 7.78 (d, J=7.4 Hz, 1H), 7.90 (s, 1H), 8.17-8.21 (m, 1H), 8.64 (d, J=4.2 Hz, 1H).

Compound 11: δ 1.05 (t, J=7.2 Hz, 3H), 1.93-2.09 (m, 6H), 2.38 (q, J=7.2 Hz, 2H), 3.01 (d, J=10.4 Hz, 2H), 3.89 (s, 3H), 3.98 (dt, J=10.8, 5.8 Hz, 1H), 7.02 (d, J=3.7 Hz, 1H), 7.45 (d, J=8.4 Hz, 1H), 7.79 (d, J=7.4 Hz, 1H), 7.89 (s, 1H), 8.18 (dd, J=3.6, 2.2 Hz, 1H), 8.63 (d, J=4.3 Hz, 1H).

Compound 12: δ 1.99 (s, 2H), 2.29-2.49 (m, 4H), 3.00-3.27 (m, 4H), 3.64 (m, 4H), 3.90 (s, 3H), 4.39 (m, 1H), 7.03 (s, 1H), 7.48 (t, J=8.0 Hz, 1H), 7.78 (d, J=7.3 Hz, 1H), 7.95 (s, 1H), 8.19 (s, 1H), 8.64 (d, J=4.2 Hz, 1H).

Compound 13: δ 1.29 (t, J=7.0 Hz, 3H), 1.91-2.14 (m, 6H), 2.23 (s, 3H), 2.88 (d, J=11.3 Hz, 2H), 3.95 (m, 1H), 4.24 (q, J=7.0 Hz, 2H), 7.03 (d, J=3.6 Hz, 1H), 7.44 (s, 1H), 7.79 (d, J=7.4 Hz, 1H), 7.90 (s, 1H), 8.18-8.2 (m, 1H), 8.64 (d, J=4.2 Hz, 1H).

Compound 14: δ 1.95-2.07 (m, 6H), 2.22 (s, 3H), 2.87 (d, J=10.7 Hz, 2H), 3.90 (s, 3H), 3.97 (s, 1H), 7.03 (d, J=3.6 Hz, 1H), 7.63 (dd, J=8.1, 1.4 Hz, 1H), 7.91 (d, J=8.1 Hz, 2H), 8.18 (s, 1H), 8.62 (d, J=4.4 Hz, 1H).

Compound 15: δ 1.29 (t, J=7.0 Hz, 3H), 1.90-2.05 (m, 6H), 2.22 (s, 3H), 2.87 (d, J=2.8 Hz, 2H), 4.01 (m, 1H), 4.24 (q, J=7.0 Hz, 2H), 7.02 (s, 1H), 7.12 (s, 1H), 7.94 (s, 1H), 8.26 (s, 1H), 8.47 (s, 1H), 8.57 (s, 1H).

Compound 16: δ 2.19 (s, 2H), 2.40 (s, 4H), 2.95-3.17 (m, 6H), 3.77 (bs, 1H), 3.83-3.94 (m, 6H), 4.23 (m, 1H), 4.48 (m, 1H), 7.41 (t, J=7.8 Hz, 2H), 7.66 (d, J=7.3 Hz, 2H), 7.98 (d, J=8.2 Hz, 2H), 8.04 (m, 2H), 8.77 (s, 2H), 9.20 (bs, 1H), 9.27 (bs, 1H).

Compound 17: δ 2.11 (s, 3H), 2.57 (m, 6H), 3.17 (bs, 2H), 3.89 (s, 3H), 4.08 (m, 1H), 7.42 (t, J=7.8 Hz, 1H), 7.69 (d, J=7.3 Hz, 1H), 7.96 (s, 1H), 7.97 (dd, J=8.3, 1.0 Hz, 1H), 8.33 (s, 1H), 8.49 (s, 1H).

### Procedure for Preparation of Compound 18

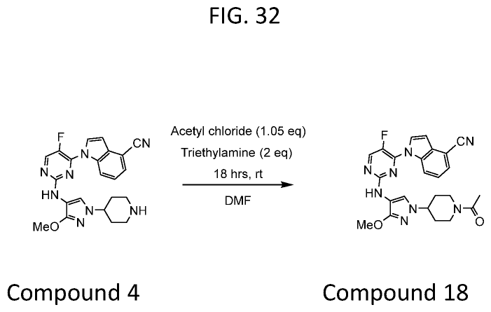

The procedure for the preparation of Compound 18 is described below and shown schematically in FIG. 32:
1. Add small molecule Compound 4 with free NH (1.00 eq) to a flask charged with DMF (3 mL).
2. Add acetyl chloride (1.05 eq) to the mixture.
3. Stir at 20° C. for 18 hours.
4. LCMS shows starting material consumed and the reaction completed.
5. Concentrate reaction mixture in vacuo to remove DMF.
6. The residue was purified by column chromatography (SiO2, Heptane/Ethyl acetate=100/0 to 0/100 followed by DCM/MeOH=100/0 to 60/40).
7. Obtain acetylated molecule Compound 18 as yellow solid.

¹H NMR: 500 MHz DMF-d7

Compound 18: δ 1.80 (qd, J=12.3, 4.5 Hz, 1H), 1.94 (qd, J=12.2, 11.5, 3.8 Hz, 1H), 2.05 (m, 2H), 2.10 (s, 3H), 2.72-2.8 (m, 1H), 3.27 (td, J=13.8, 13.1, 2.8 Hz, 1H), 3.89 (s, 3H), 4.03 (d, J=13.9 Hz, 1H), 4.30 (tt, J=11.3, 4.1 Hz, 1H), 4.58 (d, J=13.2 Hz, 1H), 7.02 (d, J=3.6 Hz, 1H), 7.45 (d, J=7.6 Hz, 1H), 7.78 (d, J=7.4 Hz, 1H), 7.93 (s, 1H), 8.17-8.2 (m, 1H), 8.64 (d, J=4.2 Hz, 1H).

General Procedure for Preparation of Intermediate 3b-11

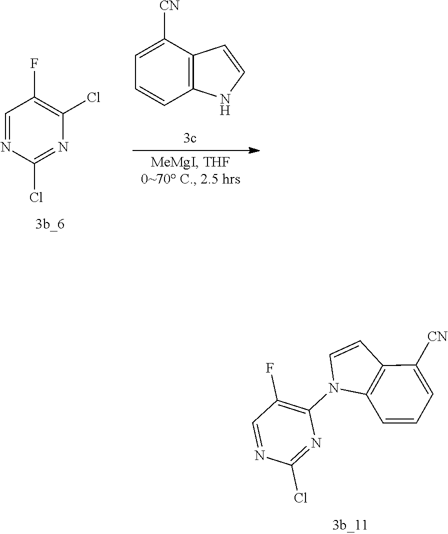

1. Add Cpd.3b_6 (6.81 g, 47.9 mmol, 1.00 eq) to a flask charged with THE (300 mL).
2. Add iodo(methyl)magnesium (3 M, 15.97 mL, 1.00 eq) to the mixture.
3. Stir at 30 min for 0° C.
4. Add Cpd.3C (8.00 g, 47.9 mmol, 1.00 eq) to the mixture.
5. Stir at 70° C. for 15 hrs
6. TLC (Petroleum ether:Ethyl acetate=3:1, Rf=0.60) shows Cpd.3b_6 consumed and the reaction completed.
7. Wash the residue with Sat. NH₄Cl until pH=7-8.
8. Extract the mixture with EtOAc (300 mL×3).
9. Wash the organic layer with brine (200 mL×2).
10. Concentrate the organic layer in vacuo to remove EtOAc.
11. The residue was purified by column chromatography (SiO₂, Petroleum ether/Ethyl acetate=30/1 to 3/1).
12. Obtain Cpd.3b_11 (5.00 g, 19.1% yield, 50% purity) as yellow solid.

### General Procedure for Preparation of Intermediate 6

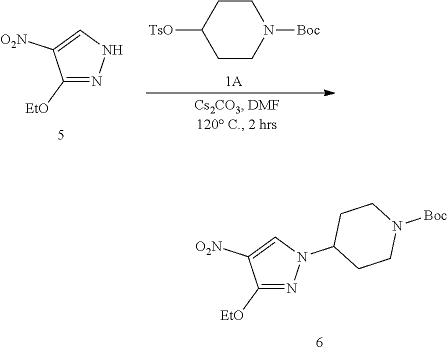 [SMILES: CCOc1n[nH]cc1[N+](=O)[O-] ; Cc1ccc(S(=O)(=O)OC2CCN(C(=O)OC(C)(C)C)CC2)cc1 ; CCOc1nn(C2CCN(C(=O)OC(C)(C)C)CC2)cc1[N+](=O)[O-]]

1. Add Cpd.5 (1.00 g, 6.36 mmol, 1.00 eq) and Cpd.1A (2.92 g, 8.21 mmol, 1.29 eq) into a flask charged with DMF (7.00 mL).
2. Add Cs₂CO₃ (3.09 g, 9.48 mmol, 1.49 eq) in the mixtures.
3. Degas with N₂ for 3 times.
4. Stir at 120° C. for 2 hrs.
5. TLC (Petroleum ether/Ethyl acetate=3/1, product Rf=0.42) shows Cpd.6 formed.
6. Adjust the PH of the mixture to 7-8 with HCl (1 M).
7. Extract with mixture with DCM (50 mL×3).
8. Concentrate in vacuo to remove DCM.
9. The residue was purified by column chromatography (SiO₂, Petroleum ether/Ethyl acetate=15/1 to 2/1).
10. Obtain Cpd.6 (1.50 g, 69.2% yield) as yellow solid.

¹H NMR: 400 MHz DMSO-d₆ δ 8.73 (s, 1H), 4.32-4.26 (m, 2H), 4.06-4.00 (m, 2H), 2.04-1.98 (m, 2H), 1.77-1.76 (m, 2H), 1.39 (s, 9H), 1.35-1.33 (m, 3H).

### General Procedure for Preparation of Intermediate 7

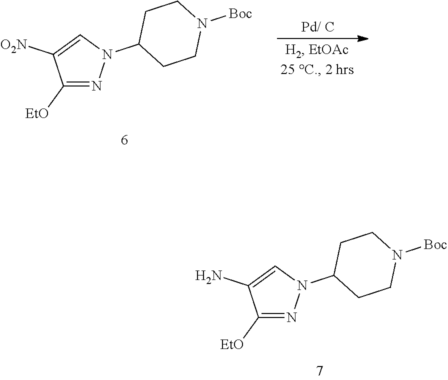 [SMILES: CCOc1nn(C2CCN(C(=O)OC(C)(C)C)CC2)cc1[N+](=O)[O-] ; CCOc1nn(C2CCN(C(=O)OC(C)(C)C)CC2)cc1N]

1. Add Cpd.6 (1.00 g, 2.94 mmol, 1.00 eq) to a flask charged with EtOAc (5 mL).
2. Add Pd/C (0.10 g, 10% purity, 1.00 eq) to the mixture.
3. Add H₂ (15 Psi) to the mixture.
4. Stir at 25° C. for 2 hrs.
5. TLC (Petroleum ether:Ethyl acetate=2:1, Rf=0.47) show Cpd.6 consumed and Cpd.7 formed.
6. Filter through celites.
7. Wash filter cake with EtOAc.
8. Concentrate the filtrate in vacuo to remove EtOAc.
9. The residue was purified by column chromatography (SiO₂, Petroleum ether/Ethyl acetate=15/1 to 2/1)
10. Obtain Cpd.7 (0.77 g, 84.4% yield) as light-yellow oil.

### General Procedure for Preparation of Intermediate 8

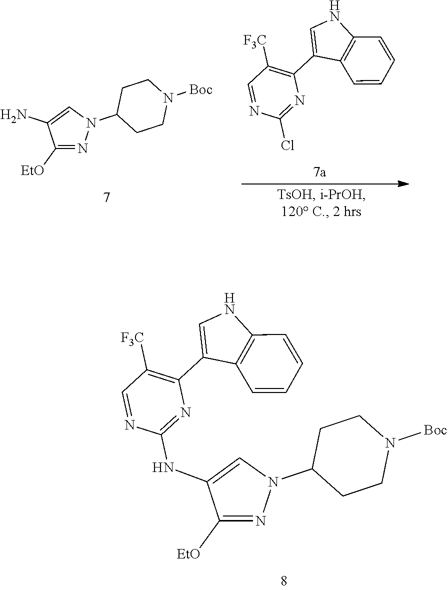 [SMILES: CCOc1nn(C2CCN(C(=O)OC(C)(C)C)CC2)cc1N ; FC(F)(F)c1cnc(Cl)nc1-c1c[nH]c2ccccc12 ; CCOc1nn(C2CCN(C(=O)OC(C)(C)C)CC2)cc1Nc1ncc(C(F)(F)F)c(-c2c[nH]c3ccccc23)n1]

1. Add Cpd.7 (0.30 g, 966 umol, 1.00 eq) and Cpd.7a (261 mg, 879 umol, 0.91 eq) charged with i-PrOH (7 mL) to a microwave vial.
2. Add TsOH (302 mg, 1.76 mmol, 1.82 eq) to the mixture.
3. Stir at 120° C. for 2 hrs.
4. LCMS (ET37912-42-p1a) shows Cpd.8 formed.
5. Adjust the pH of the mixture to 7˜8 with Sat. NaHCO₃.
6. Extract the mixture with EtOAc (60 mL×3).
7. Wash the organic layer with brine (30 mL×2).
8. Concentrate the organic in vacuo to remove EtOAc.
9. Obtain Cpd.8 (0.3 g, crude) as yellow solid.

### General Procedure for Preparation of Compound 7

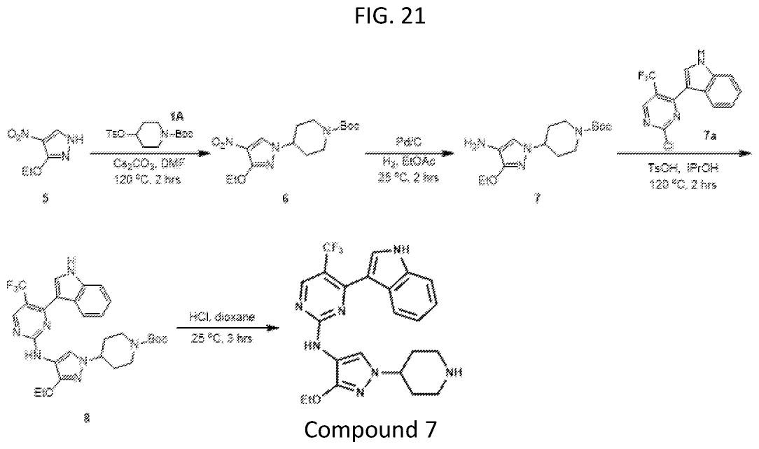

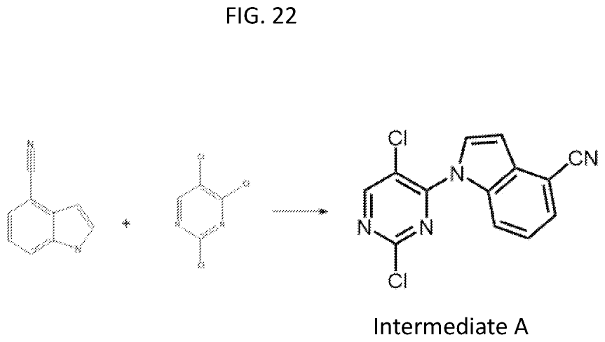

The procedure for the preparation of Compound 7 is shown schematically in FIG. 21.
1. Dissolve Cpd.8 (0.50 g, 874 umol, 1.00 eq) in HCl/dioxane (4 M, 5.00 mL, 22.8 eq).
2. Stir for 3 h at 20° C.
3. TLC (Petroleum ether/Ethyl acetate=5/1, product Rf=0.1) shows Compound 7 formed.
4. Add sat. NaHCO₃ (50.0 mL) to the solution, pH=8.
5. Extract with EtOAc (50.0 mL×2), separate the organic layer and dry over Na₂SO₄, concentrate in vacuum.
6. The crude product was triturated with EtOAc (100 mL) at 25° C. for 30 min.
7. Filter and collect the cake.
8. Obtain Compound 7 (0.068 g 16.0% yield) as white solid.

¹H NMR: 400 MHz DMSO-d₆ δ 9.03-8.88 (s, 1H), 8.60 (s, 1H), 8.52-7.96 (s, 1H), 7.85 (s, 1H), 7.74 (s, 1H), 7.45-7.43 (s, 1H), 7.10-6.96 (m, 2H), 4.30 (s, 2H), 3.85 (s, 1H), 3.02-2.99 (m, 2H), 2.66-2.55 (m, 2H), 1.88 (m, 2H), 1.70-1.67 (m, 2H), 1.26-1.17 (m, 3H).

### General Procedure for Preparation of Intermediate A

3M MgICH3 (1.781 mL, 5.34 mmol) was added to a yellow solution of 1J-indole-4-carbonitrile (0.760 g, 5.34 mmol) in TFH (30 mL). The yellow suspension turned to a while solid, which was formed indole Mg complex. After 15 min, 2,4,5-trichloropyrimidine (1 g, 5.34 mmol) was added at 0° C., the mixture was warmed to room temperature. LCMS showed slight product. The mixture was heated to 70° C. for 2 hours, the reaction progressive. After stirring overnight, the reaction completed. Product mass was [M+1}: 288.9 at retention time RT=3.05 min. The reaction was kept overnight at 70° C. The reaction wasn't completed. Filtered off solid to give a crude product 3-(2,5-dichloropyrimidin-4-yl)-1H-indole-4-carbonitrile (0.546 g, 35.0%). 25 mg crude compound was taken, purified by Gilson, UV lamp was 222 nm, mobile phase by 25-80% CAN in 0.1 TFA in water to give 17 mg while solid product was a TFA salt. LCMS and NMR confirmed the target compound. 1H NMR (300 MHz, DMSO-d₆): δ 6.94 (d, J-3.58 Hz, 1H) 7.32-7.53 (m, 1H) 7.72 (d, J=7.54 Hz, 1H) 8.09 (dd, J=5.93, 2.35 Hz, 2H) 9.09 (s, 1H).

### Example 3. Cells and Culture Methods

All of the stable cell lines generated from HeLa and Huh7 cells were maintained and cultivated in Dulbecco's modified Eagle's medium (DMEM) with high glucose with GlutaMAX (Gibco, cat. no. 31966-021), 10% fetal bovine serum (gibco, cat. no. 10270-106), and 1% penicillin-streptomycin (gibco, cat. no. 15140-122) at 37° C., 5% CO₂ in 95% humidity in T75 TC flask (Sarstedt, 83.3911.002). The cells were detached (trypsin 0.25% EDTA, 10 min) and passaged twice a week. HEK293 and HeLa, HepG2, Huh7 and U2OS mCherry-GAL9 cell lines were maintained in DMEM, NCI-H358 mCherry-GAL9 were maintained in RPMI (Gibco: 32430-027) and SH-SY5Y mCherry-GAL9 were maintained in DMEM/F12 (Gibco: 32430-027). All growth media was supplemented with 10% FBS and 1 μg/ml Puromycin (Gibco: A11138-03) to maintain reporter expression. All cells were routinely tested and mycoplasma negative.

### Example 4. Splice Switching Activity Assays

### Example 4A Splice Switching Activity Assays in HeLa Luc705 and Huh7 Luc705 Cells

HeLa Luc705 and Huh7 Luc705 cells were used to screen and characterize the SMC generated in Example 2 for their ability to increase levels of an SSO-induced splice variant by co-treatment of the cells with the SSO and SMC. The Luc705 reporter construct was used for quantitation of luciferase protein produced as a result of functional SSO delivery. (Kang et al. (1998) Biochemistry, 37(18):6235-6239; Rocha et al. (2016) Four Novel Splice-Switch Reporter Cell Lines: Distinct Impact of Oligonucleotide Chemistry and Delivery Vector on Biological Activity. Nucleic Acid Ther. 26(6):381). Briefly, a plasmid carrying the luciferase coding sequence interrupted by an insertion of intron 2 from β-globin pre-mRNA carrying a cryptic splice site was stably transfected into cells. Unless the aberrant slice site was masked by an antisense oligonucleotide, the pre-mRNA of luciferase was improperly processed.

UNC2383 is a small molecule compound described by Wang et al. that enhances the pharmacological effectiveness of antisense and splice switching oligonucleotides. (Wang et al. (2017) ACS Chem. Biol. 12(8):1999-2007).

HeLa Luc705 and Huh7 Luc705 cells were seeded in 96-well plates at 10,000 cells per well in growth media containing 1 μM Luc705-SSO and incubated to allow adherence and oligo internalization. After 24 hours, 10 μl of SMC diluted in Growth Media (DMEM+10% FBS) was added into each well. After 2 hours, media was removed from the wells and replaced with fresh growth media for 4 hours to allow for translation of the variant proteins. The media was then removed, and cells were lysed with 0.1% Triton-PBS.

To detect luciferase activity, 30 μL of cell lysate was transferred to white-walled 96-well plates. Luciferase intensity in each well was measured using a GloMax® 96 Microplate Luminometer machine (Promega) following auto-injection of 25 μL Luciferin substrate (Firefly Luciferase Assay System: Promega) per well. Photon measurements were acquired for 10 seconds, starting 2 seconds after injection.

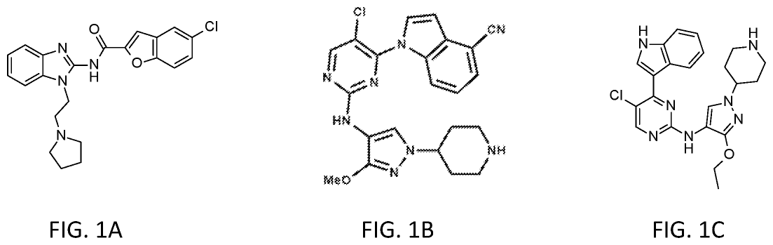

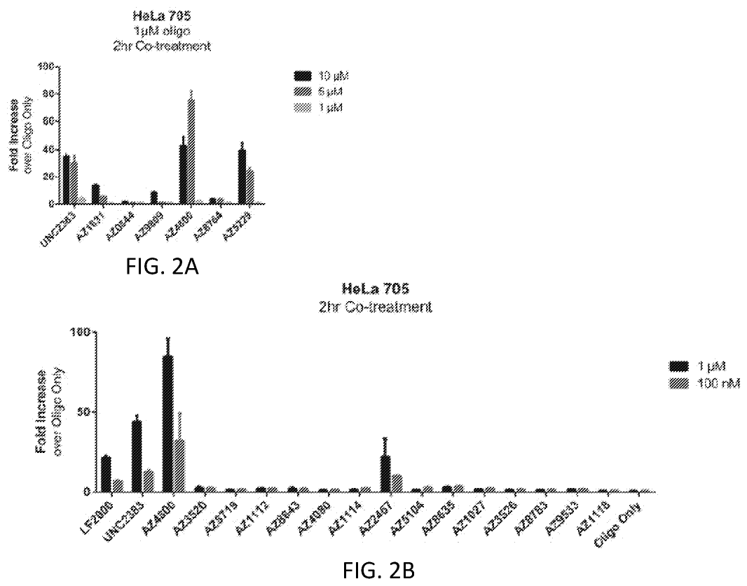

As shown in FIG. 2A, AZ4800 (Compound 2, FIG. 1B) induced a substantially higher increase in SSO activity than UNC2383 (FIG. 1A).

As shown in FIG. 2B, select compounds were tested essentially as described above in connection with 1 μM SSO and 100 nM SSO cotreatment to identify compounds that are effective with sub-micromolar SSO concentrations. Most of the compounds did not induce an increase in activity, aside from AZ4800 (Compound 2, FIG. 1B) and AZ2467 (Compound 6, (FIG. 1C).

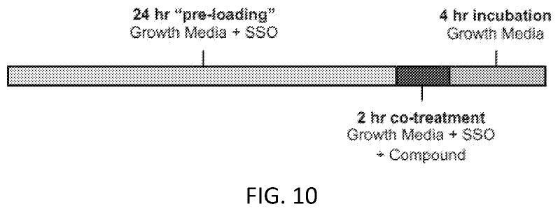

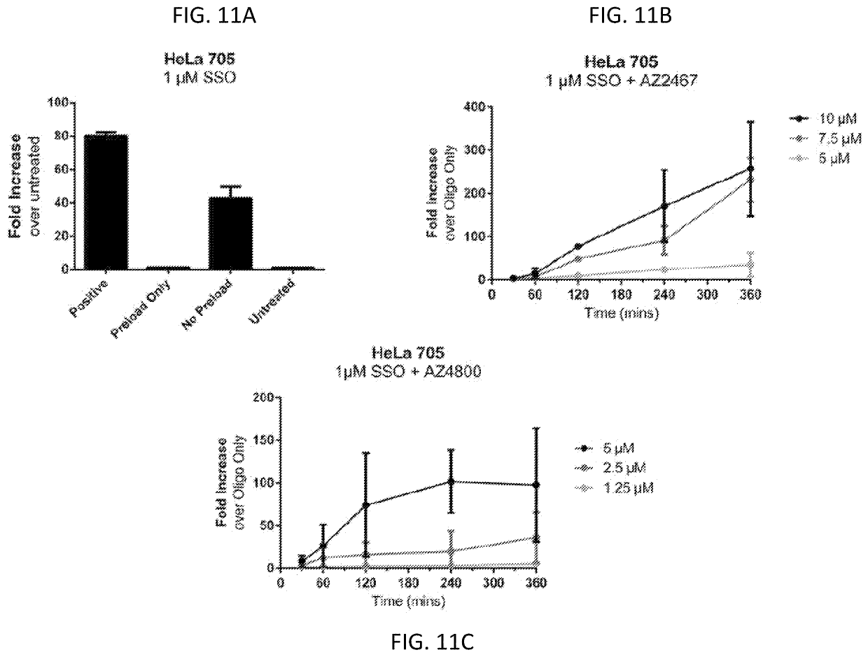

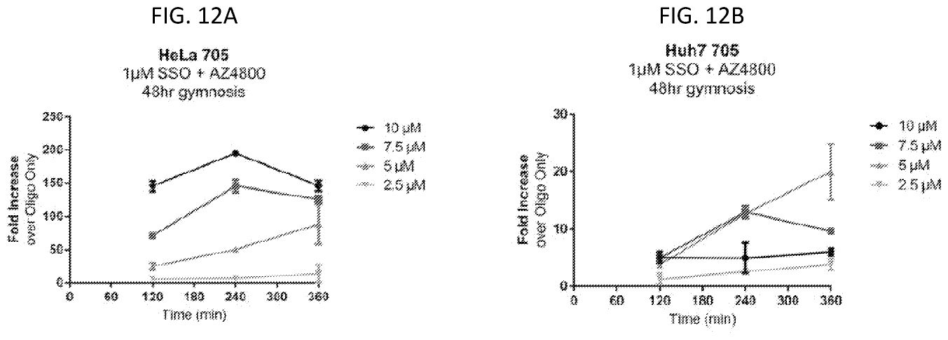

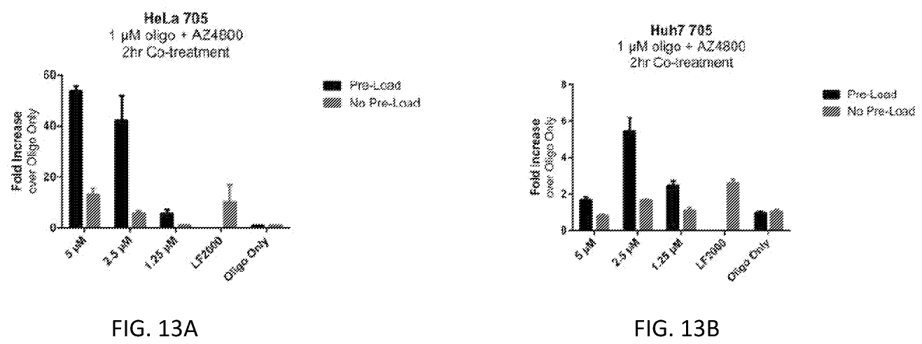

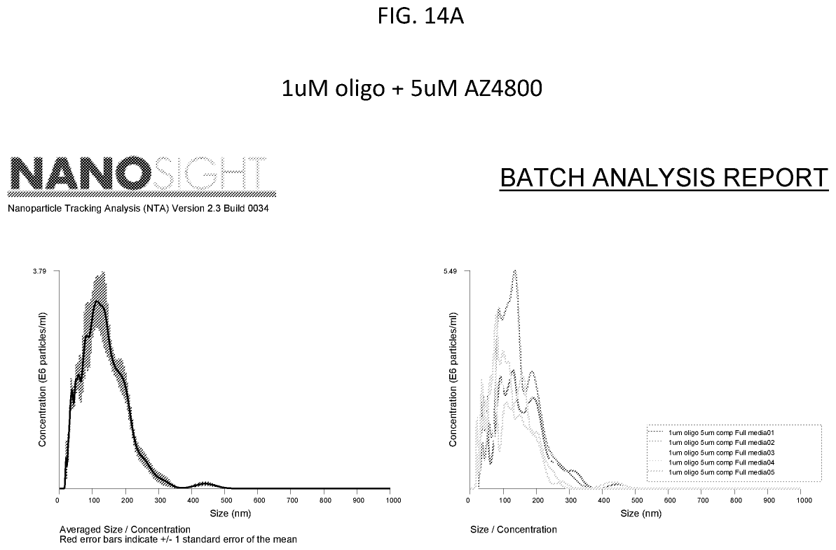

To determine whether AZ4800 (Compound 2, FIG. 1B) and SSO must be present in the cell media simultaneously for Compound 2 to produce its synergistic effects, the treatment plan was modified from the UNC2383 treatment plan detailed by Wang et al. (2017) ACS Chem. Biol. 12(8):1999-2007 to include a 24 hour pre-treatment with SSO, followed by the addition of Compound 2 for two hours, followed by the removal of SSO and Compound 2, and finally a 4-hour protein translation period (FIG. 10). When the pre-treatment step was lengthened to 48 hours, increase in SSO activity was more pronounced (FIGS. 12A and 12B). Importantly, when SSO was only pre-treated, but then removed when compound was added, no activity was seen (FIG. 11A). Importantly, significant activity was observed even without the pre-treating step (FIGS. 13A and 13B).

Using the Luc705 model, functional concentration and time curves were generated for both Compounds 2 (AZ4800) and 6 (AZ2467). See FIGS. 11B and 11C. Co-treatment with Compound 2 (AZ4800) increased SSO activity up to 5 μM, leading to a 100-fold increase in activity and then plateauing after 2 hours. Compound 6 (AZ2467) led to a 200-fold increase in SSO activity up to 6 hours at 10 μM.

### Example 4B Splice Switching Activity Assays in HeLa Luc705 Cells

HeLa_Luc705 cells were seeded in 96-well plates at 10,000 cells per well in growth media with 2 μM Luc705-SSO or 1 μM Luc705-SSO. The various SMCs were diluted directly into prepared growth media. After 24 hr to allow adherence and SSO internalization, 10 μl of diluted SMC was added into each well at indicated concentrations. For a positive control, SSOs were complexed with Lipofectamine 2000 for 30 min at RT and cells treated with a final concentration of 200 nM. ON concentrations above 200 nM would require LF2000 to be used in excess of the cells' toxicity threshold. After 24 hr treatment, cells were lysed with 0.1% TritonX-100 (Sigma Aldrich, cat no. X100) in 1× PBS (Gibco, cat no. 10010023).

For the detection of luciferase activity, 30 μL of cell lysate was transferred to white-walled plates. The luciferase intensity in each well was immediately measured via luminometer following auto-injection of 25 μL Luciferin substrate as per the Promega Firefly Luciferase Assay System. The timing parameters of photon measurements were optimized for this procedure.

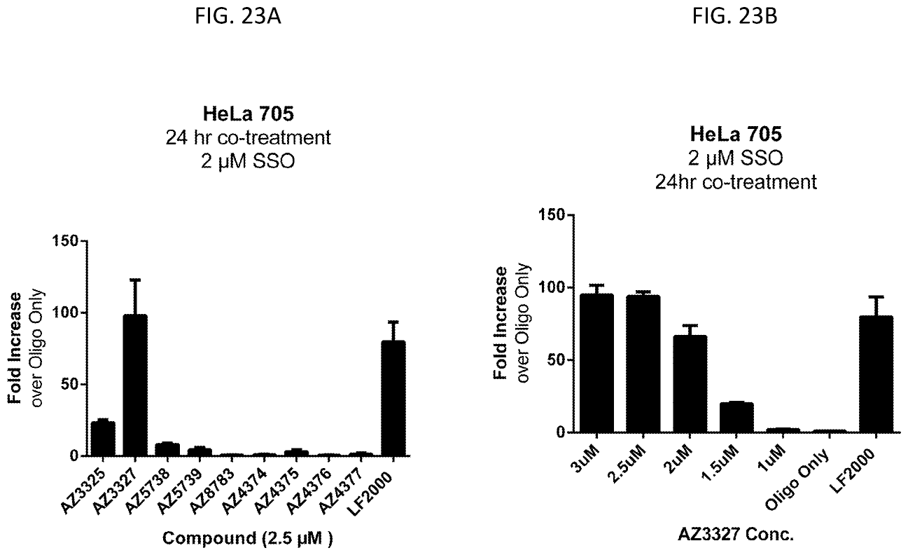

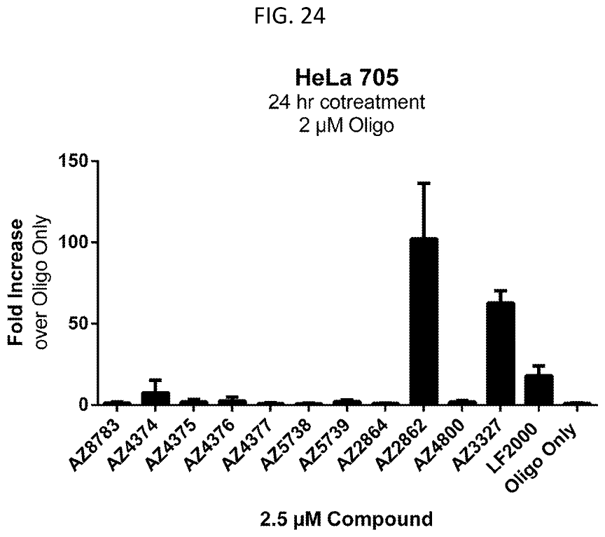

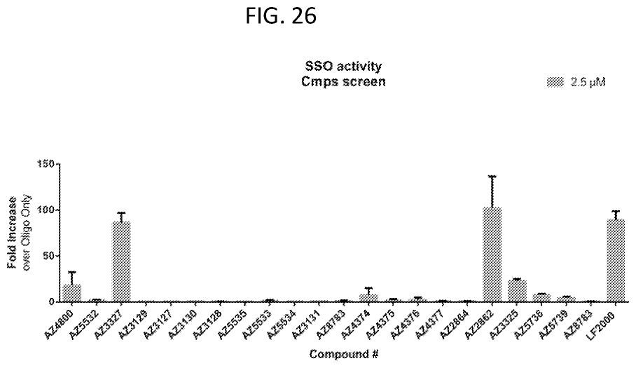

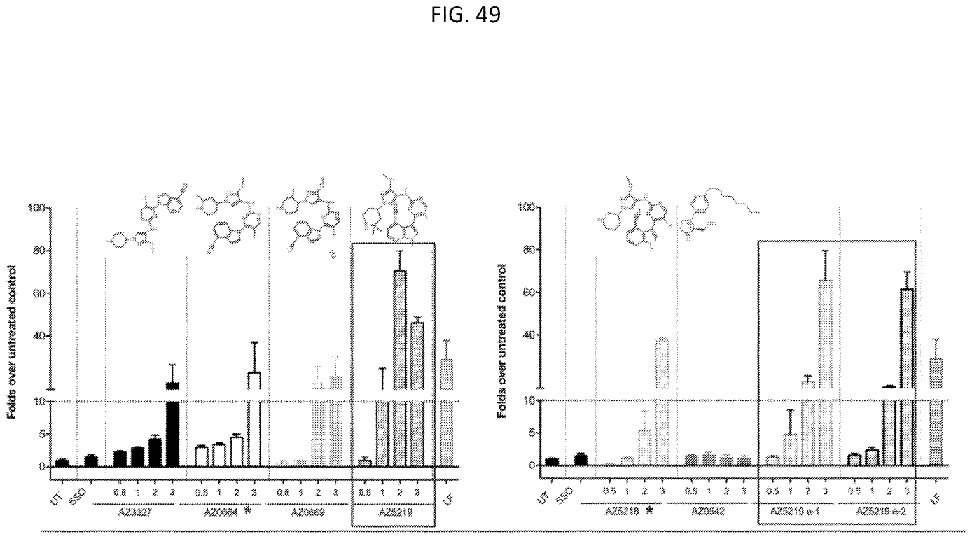

As shown in FIG. 23A, select compounds were tested essentially as described above in connection with 2 μM SSO in HeLa 705 cells to identify compounds that are effective with SSO. Most of the compounds did not induce an increase in SSO activity, aside from AZ3325 (Compound 7) and AZ3327 (Compound 4). As shown in FIG. 23B, higher concentrations of AZ3327 (Compound 4) led to an increase in SSO activity over 1 μM naked SSO in HeLa 705 cells. As shown in FIG. 24, select compounds were tested essentially as described above in connection with 2 μM Luc 705 SSO in HeLa 705 cells to identify compounds that are effective with SSO. Most of the compounds did not induce an increase in SSO activity, aside from AZ4374 (Compound 21), AZ2862 (Compound 8) and AZ3327 (Compound 4). As shown in FIG. 26, select compounds were tested essentially as described above in connection with 2 μM Luc 705 SSO in HeLa 705 cells to identify compounds that are effective with SSO. Several compounds induced an increase in SSO activity, including AZ4800 (Compound 2), AZ3327 (Compound 4), AZ4374 (Compound 21), AZ2862 (Compound 8) and AZ3325 (Compound 7). FIG. 49 shows results of combining AZ5219 (Compound 32) with 1 μM Luc 705 SSO in HeLa 705 cells at various time intervals.

### Example 4C Splice Switching Activity Assays in U2OS_Luc705 and N2A_Luc705 Cells

U2OS_Luc705 and N2A_Luc705 cells were seeded in 96-well plates at 10,000 cells per well in growth media with 1 μM Luc705-SSO. The various SMCs were diluted directly into prepared growth media. After 24 hr to allow adherence and SSO internalization, 10 μl of diluted SMC was added into each well at indicated concentrations. For a positive control, SSOs were complexed with Lipofectamine 2000 for 30 min at RT and cells treated with a final concentration of 200 nM. ON concentrations above 200 nM would require LF2000 to be used in excess of the cells' toxicity threshold. After 24 hr treatment, cells were lysed with 0.1% TritonX-100 (Sigma Aldrich, cat no. X100) in 1× PBS (Gibco, cat no. 10010023).

For the detection of luciferase activity, 30 μL of cell lysate was transferred to white-walled plates. The luciferase intensity in each well was immediately measured via luminometer following auto-injection of 25 μL Luciferin substrate as per the Promega Firefly Luciferase Assay System. The timing parameters of photon measurements were optimized for this procedure.

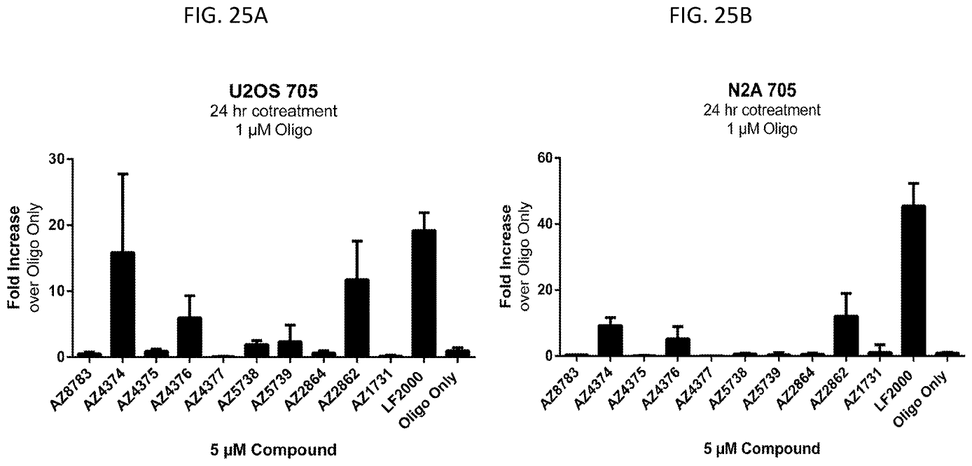

As shown in FIG. 25A, select compounds at 5 μM were tested essentially as described above in connection with 1 μM Luc 705 SSO in U2OS_Luc705 cells to identify compounds that are effective with SSO. AZ4374 (Compound 21), AZ4376 (Compound 22), Compound AZ5738 (Compound 25), AZ5739 (Compound 20), and AZ2862 (Compound 8) induced an increase in SSO activity relative to oligo only. As shown in FIG. 25B, select compounds at 5 μM were tested essentially as described above in connection with 1 μM Luc 705 SSO in N2A 705 cells to identify compounds that are effective with SSO. AZ4374 (Compound 21), AZ4376 (Compound 22), and AZ2862 (Compound 8) induced an increase in SSO activity relative to oligo only Example 4D Splice Switching Activity Assays in HeLa Luc705 cells, U2OS_Luc705 cells and N2A_Luc705 cells HeLa_Luc705, U2OS_Luc705, and N2A_Luc705 cells were seeded in 96-well plates at 10,000 cells per well in growth media with 1 μM Luc705-SSO. The various SMCs were diluted directly into prepared growth media. After 24 hr to allow adherence and SSO internalization, 10 μl of diluted SMC was added into each well at indicated concentrations. For a positive control, SSOs were complexed with Lipofectamine 2000 for 30 min at RT and cells treated with a final concentration of 200 nM. ON concentrations above 200 nM would require LF2000 to be used in excess of the cells' toxicity threshold. After 24 hr treatment, cells were lysed with 0.1% TritonX-100 (Sigma Aldrich, cat no. X100) in 1× PBS (Gibco, cat no. 10010023).

For the detection of luciferase activity, 30 μL of cell lysate was transferred to white-walled plates. The luciferase intensity in each well was immediately measured via luminometer following auto-injection of 25 μL Luciferin substrate as per the Promega Firefly Luciferase Assay System. The timing parameters of photon measurements were optimized for this procedure.

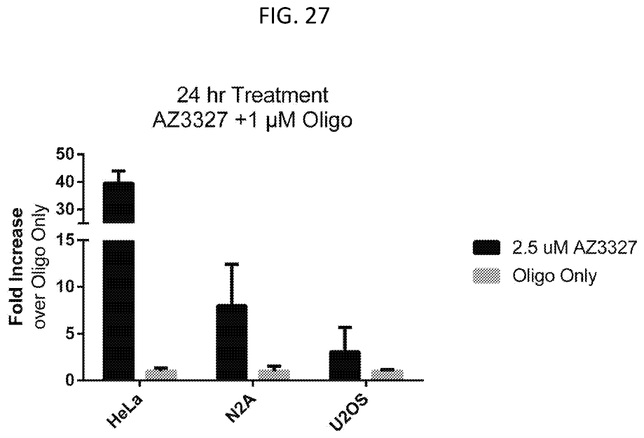

As shown in FIG. 27, 2.5 μM of AZ3327 (Compound 4) was tested essentially as described above in connection with 1 μM SSO in three cell types (HeLa_Luc705, U2OS_Luc705, and N2A_Luc705 cells) to demonstrate enhanced activity of the oligo as compared to oligo only. We can conclude that the oligo-enhancing activity demonstrated by these compounds is not necessarily dependent on cell type.

### Example 5. Reverse-Transcription Polymerase Chain Reaction (RT-PCR)

The percentage of corrected luciferase mRNA was quantified using a previously validated RT-PCR protocol. (Saher et al. (2019) Pharmaceutics. 11(12):666) Total RNA was extracted from HeLa Luc705 and Huh7 Luc705 cells using TRI Reagent (Sigma-Aldrich) according to the manufacturer's instructions.

For RT-PCR reactions using ONE STEP RT-PCR kit (QIAGEN), three nanograms of isolated RNA were utilized. The total reaction volume was 20 μL and the primer sequences used were:

Fwd-
(SEQ ID NO: 2)
5′-TTGATATGTGGATTTCGAGTCGTC-3′
Rev-
(SEQ ID NO: 3)
5′-TGTCAATCAGAGTGCTTTTGGCG-3′

The program for the RT-PCR was as follows: 55° C., 35 min, then 95° C., 15 min, for the reverse transcription step, directly followed by the PCR (94° C., 30 s, then 55° C., 30 s, then 72° C., 30 s) for 30 cycles and the final extension 72° C., 10 min. The PCR products were analyzed using a 1% agarose gel in 0.5× TAE buffer and visualized by SYBR Gold (Invitrogen, Molecular products) staining.

Versadoc imaging system with a cooled CCD camera (BioRad, Hercules, CA, USA) was used for the analysis of gels. Band intensities were analyzed with the Quantity One software (BioRad, Hercules, CA, USA). The percentage of correction was calculated using this equation (Band intensity of corrected RNA*100/(Band intensity of corrected RNA+Band intensity of uncorrected RNA).

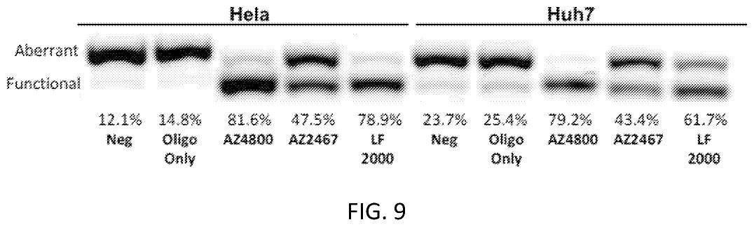

RT-PCR confirmed that the increase in functional luciferase production was the result of antisense SSO activity on the target Luc705 pre-mRNA. Quantitation of the mRNA splice variants revealed increases in the percentage of alternatively-spliced mRNA (FIG. 9). Notably, treatment with AZ4800 (Compound 2) and SSO was shown to induce greater levels of splice-switching than transfection with SSO using Lipofectamine 2000, a widely used transfection reagent, in both HeLa and Huh7 cells.

### Example 6. mCherry-GAL9 Recruitment Assay

All compounds were subjected to a mCherry-GAL9 recruitment assay. Under normal conditions, mCherry-GAL9 is homogenously dispersed in the cytosol. When an endosomal leakage event is detected, mCherry-GAL9 translocates to the damaged endosome (Du Rietz et al. (2020) Nat. Commun. 11:1809) The resulting localization pattern of discrete puncta can be visualized and quantitated.

HeLa and Huh7 cell lines stably expressing mCherry-GAL9 were generated as described by (bioRxiv) (Munson, M. et al. (2021) Communications Biology, 4(211): 1-14) and seeded into 384-well CellCarrier Ultra plates (PerkinElmer: 6007558) at 3000 or 3500 cells/well respectively, 16 h before experimental usage.

Dose-response curves of select compounds were generated using an Echo 655T acoustic dispenser (Labcyte) to dispense the indicated compounds into source plates (Greiner: 781280) containing growth media. At experimental start points, media containing appropriate compounds and doses was transferred to plates using a liquid handling robot (Agilent Bravo).

At assay endpoints, cells were washed 2× PBS at RT and fixed in 4% PFA (VWR: 9713.1000) for 15 mins/RT. Cells were washed a further 3× PBS before the addition of PBS+1 μg/ml Hoechst 33342 (ThermoFisher Scientific: H21492) for a minimum of 1 h before imaging.

Plates were imaged using a spinning-disk confocal microscope (Yokogawa: CV7000) with a 20× objective (NA 0.75). Images were processed utilizing Columbus image-analysis software (PerkinElmer: v2.9.0) to identify and quantify cells and mCherry-GAL9 structures. The resulting data were processed and normalized in Spotfire (Tibco: v10.3) and plotted in Prism (Graphpad: v8.0.1). Image panels were assembled using the FigureJ plugin for FIJI. (Mutterer and Zinck (2013) J. Microsc. 252(1):89-91).

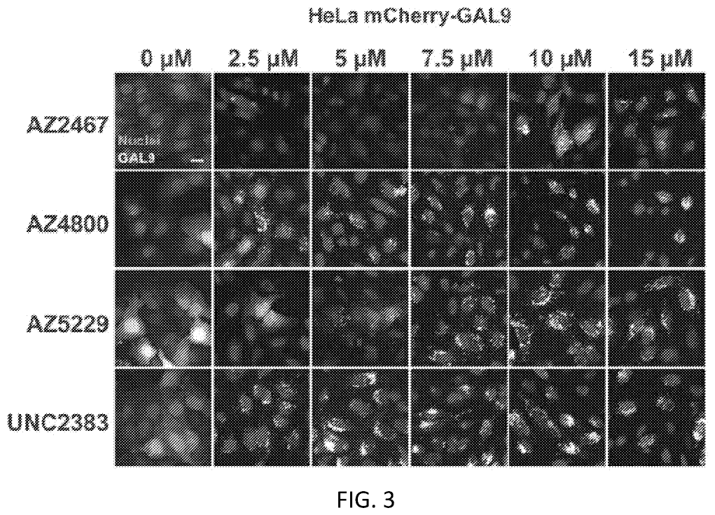

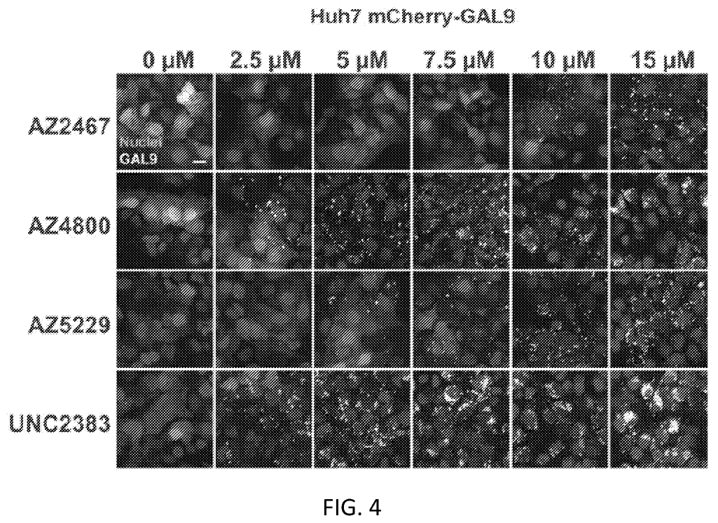

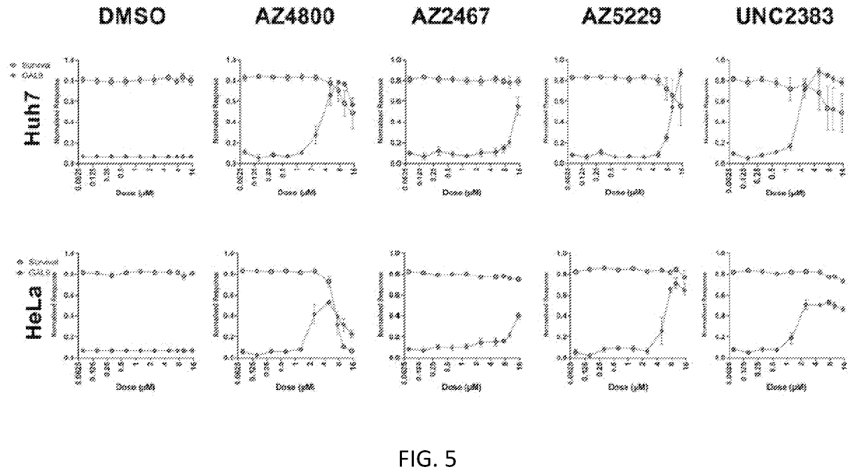

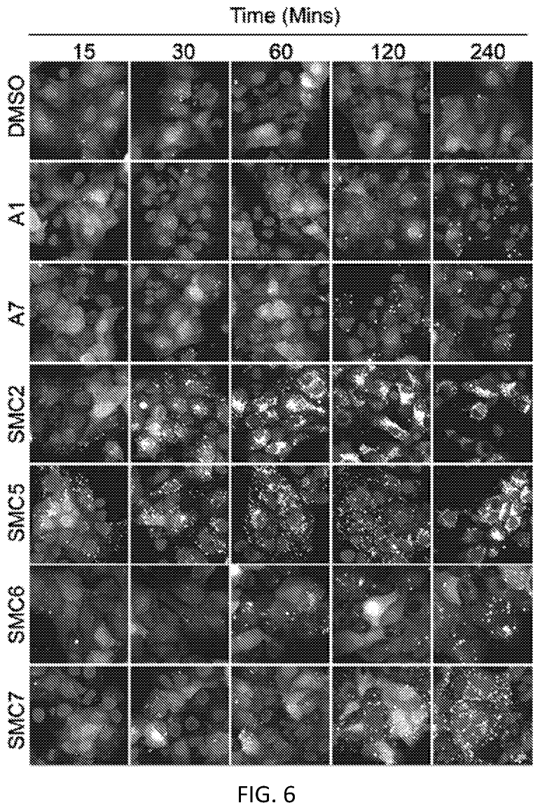

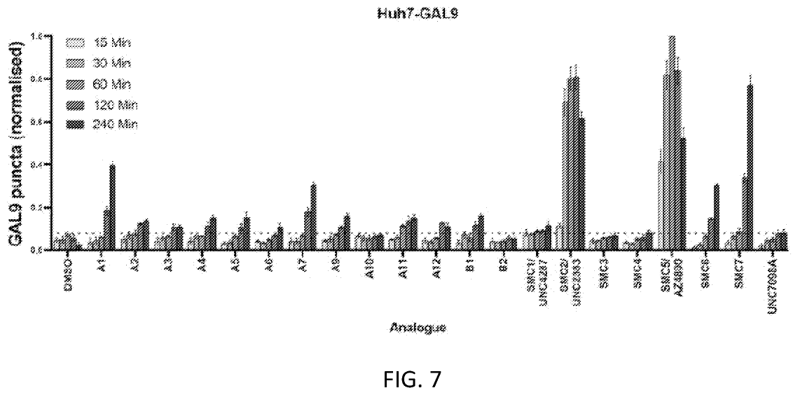

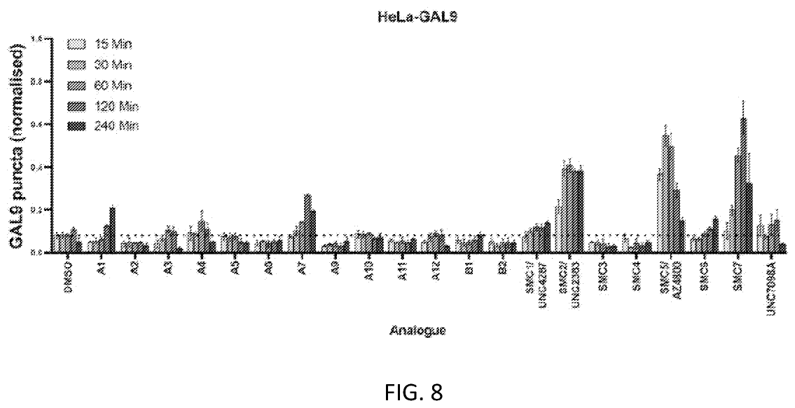

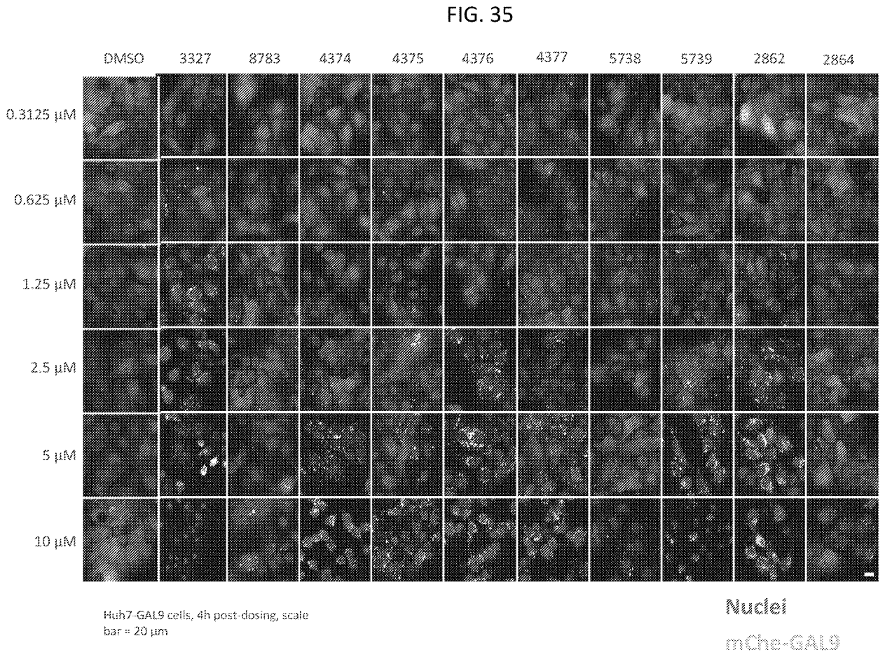

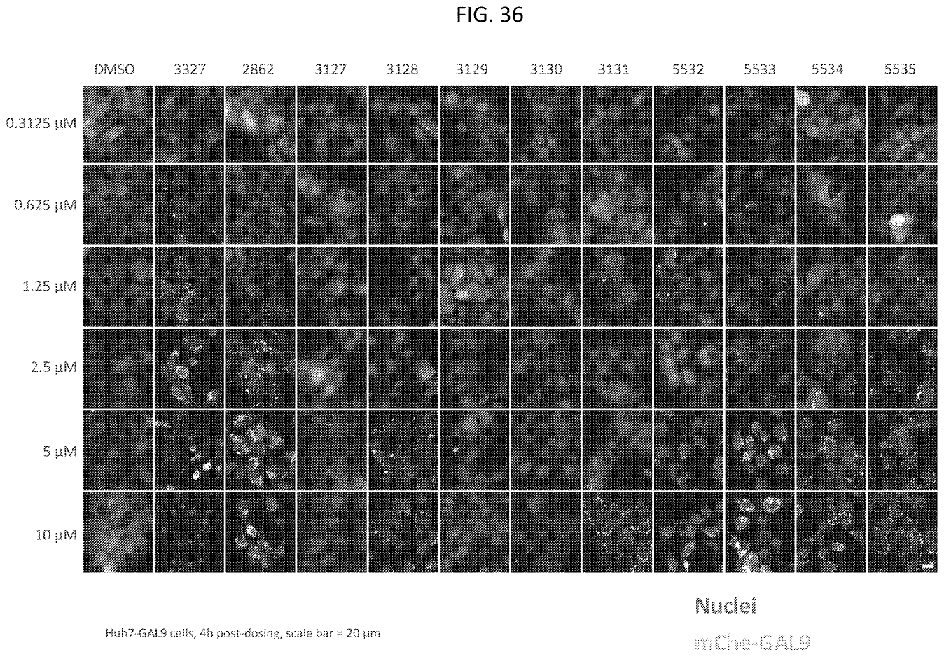

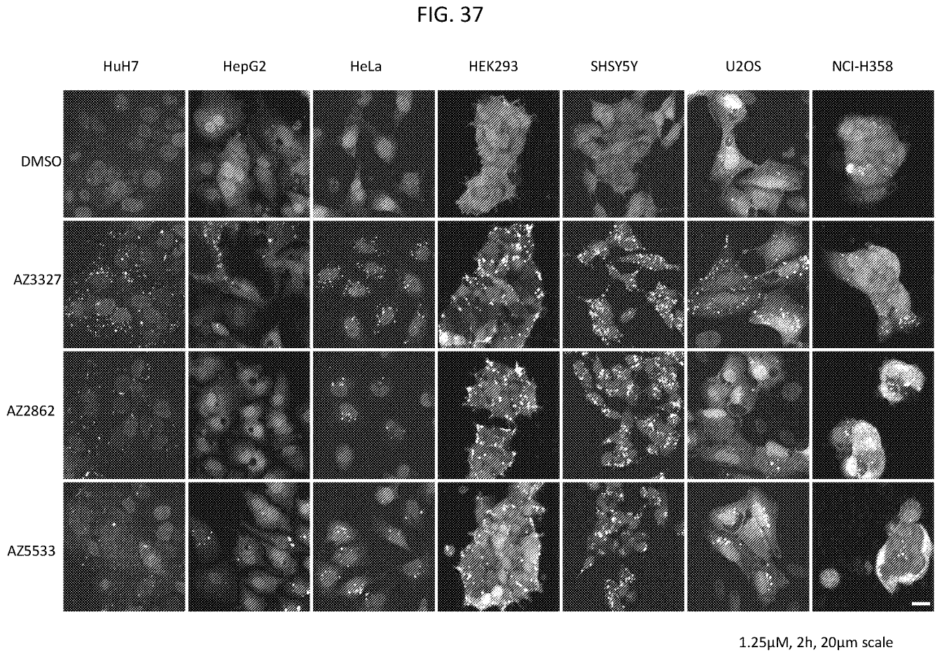

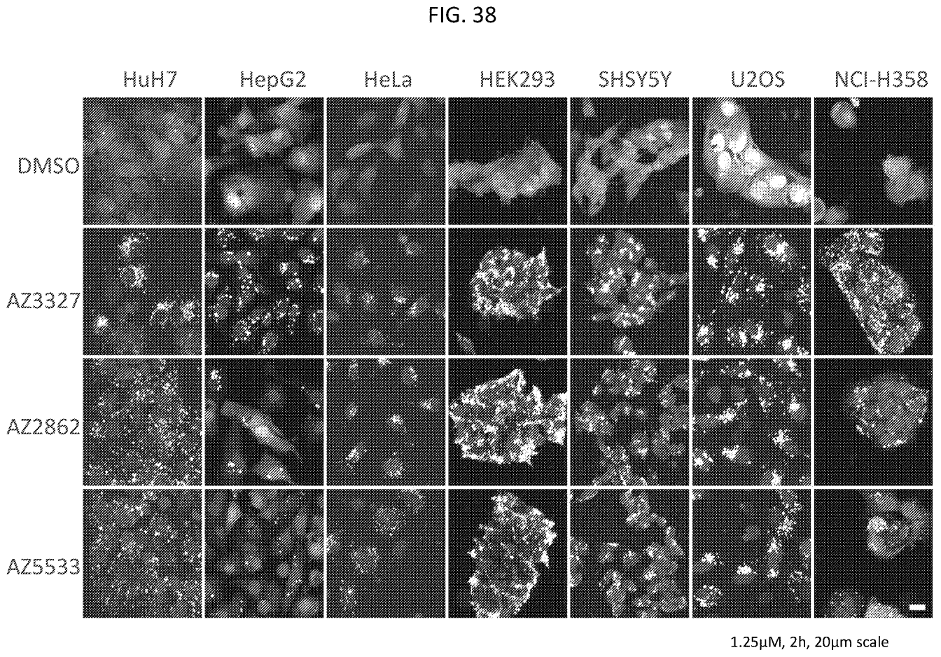

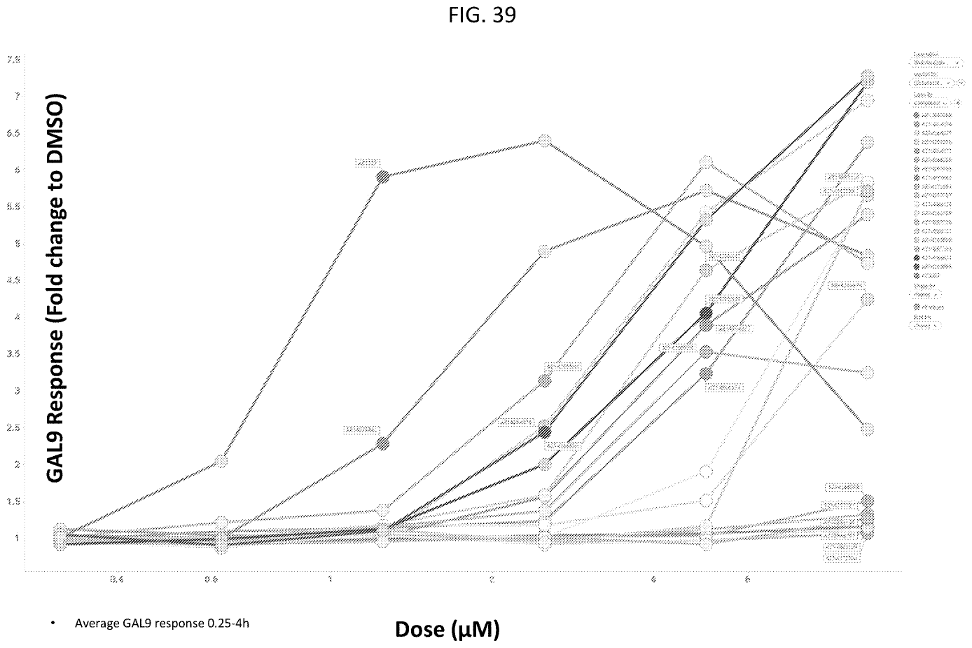

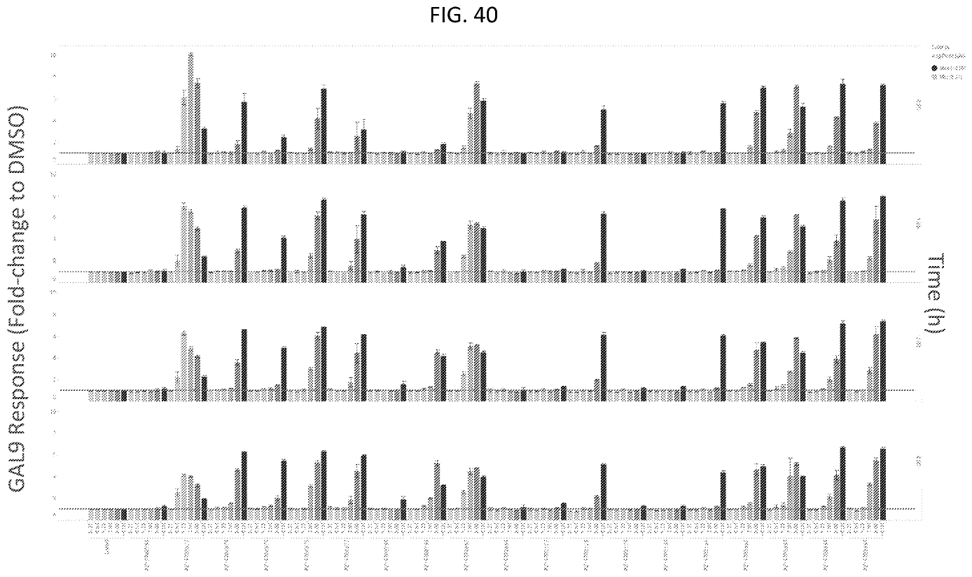

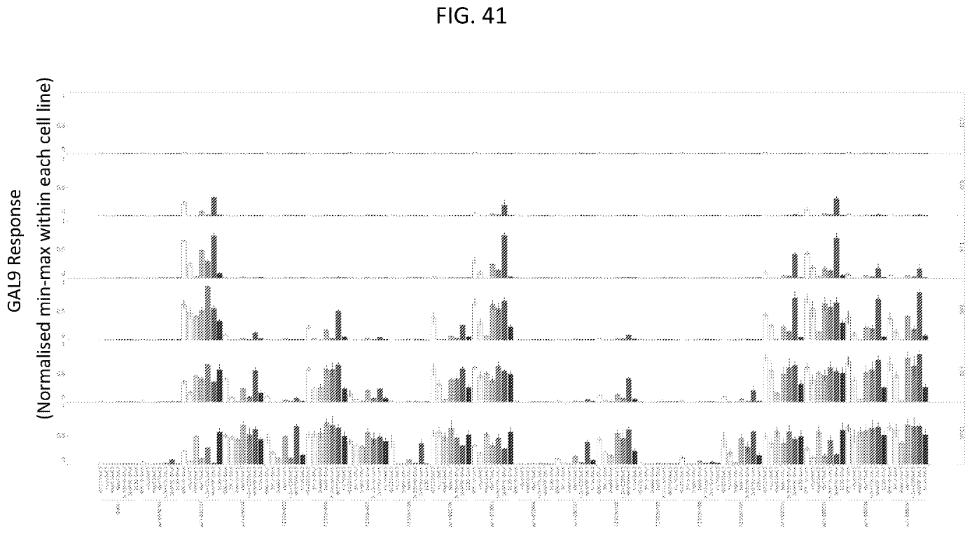

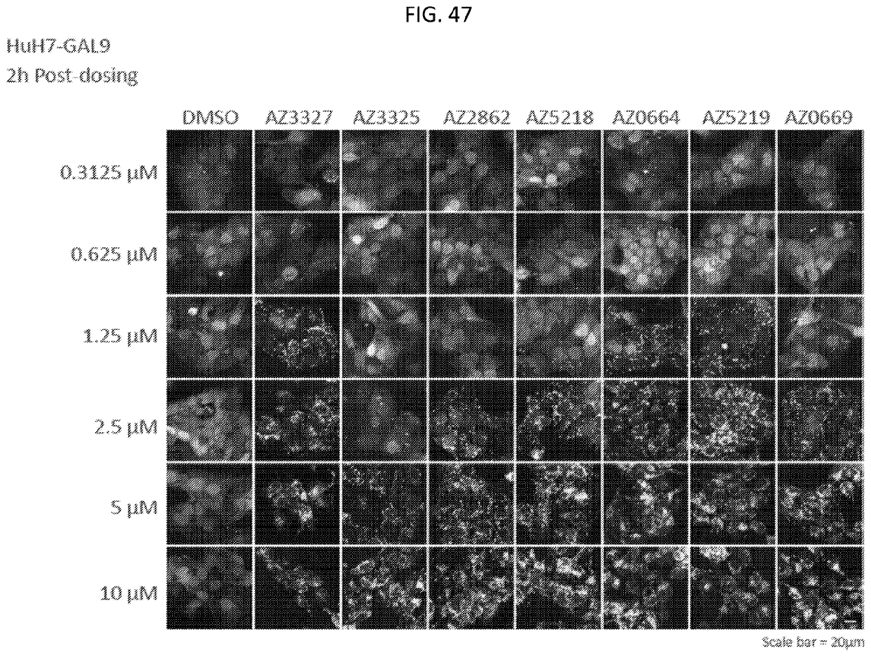

Cells were subjected to a 10-point concentration dilution (0-15 μM) for 2 hours and then fixed and imaged (FIG. 3 and FIG. 4). Images were analyzed and curves were generated from both mCherry-GAL9 puncta quantitation and cell survival as determined by nuclear morphology (FIG. 5). The GAL9 recruitment response support the functional splice-switching data with SSOs, confirming the mCherry-GAL9 assay as a powerful tool for screening endosomolytic compounds and supporting the hypothesis that AZ2467 (Compound 6) and AZ4800 (Compound 2) promote SSO activity by inducing endosomal rupture. The GAL9 assay was again conducted, this time in live cells for 4 hours with select compounds for screening. Compounds were screened at 5 μM (FIG. 6-8) or 0.3125-10 μM (FIG. 35-38). FIG. 39 and FIG. 47 provide results for select compounds at various doses 0.3125-10 μM.

This assay revealed that within 4 hours, GAL9 translocation was activated for several of the compounds. Compounds SMC5 (Compound 2), A7 (Compound 6), and A1 (Compound 5) were selected for further characterization due to their efficacy at shorter-term timepoints.

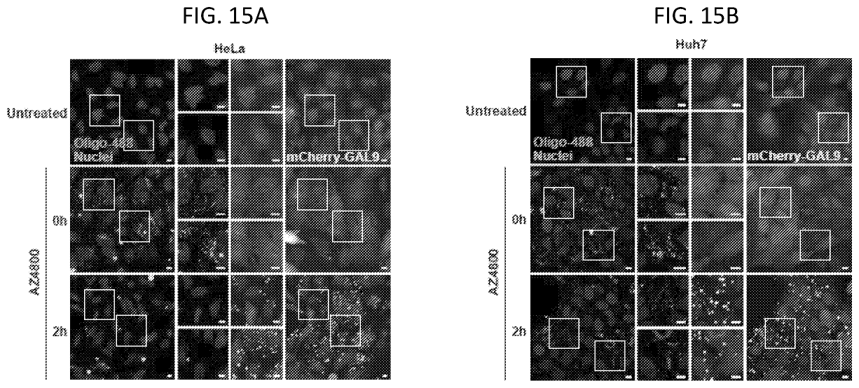

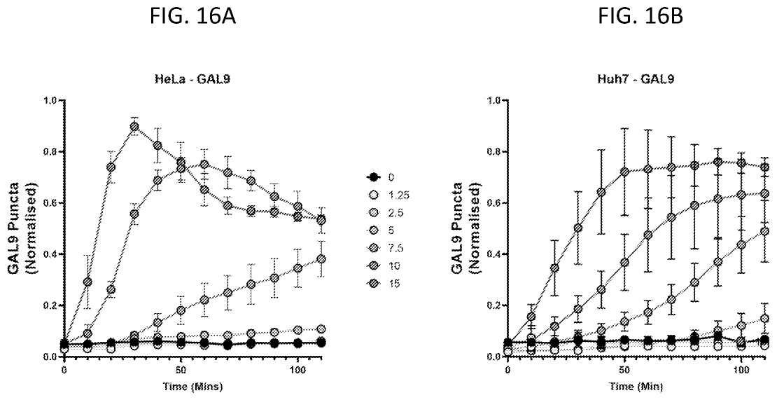

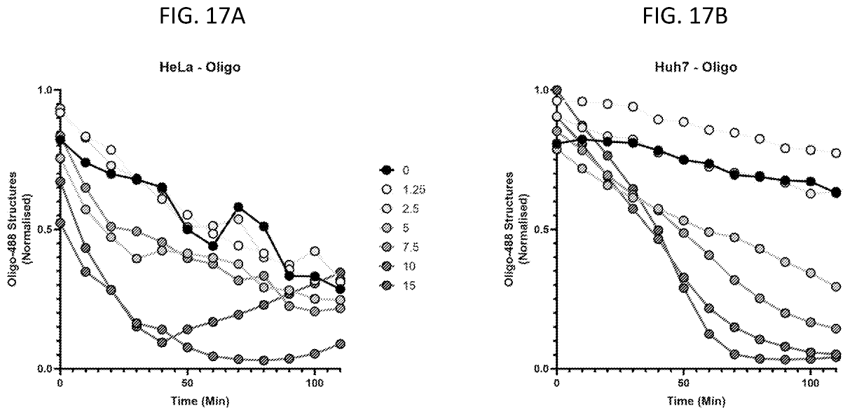

HeLa and Huh7 cells were submitted to the mCherry-GAL9-recruitment analysis with co-treatment with AZ4800 (Compound 2) and 1 μM A488-SSO. After preloading A488-SSO, A488-positive structures that likely reflect endosomal localization could be detected. These A488-positive structures gradually disappeared over 2 hours following treatment with AZ4800 (Compound 2), indicating endosomal rupture and subsequent leakage of A488-SSO into the cytosol where it is too diffuse to detect (FIGS. 15A and 15B). This rupture occurred in a time and concentration-dependent manner, consistent with earlier functional AZ4800 (Compound 2) findings (FIGS. 16A and 16B). Simultaneously, the GAL9 re-localization resulted in the formation of mCherry-GAL9 puncta (FIGS. 17A and 17B). The quantitation of GAL9 also revealed time and concentration-dependent responses, supporting the hypothesis that A488-SSO endosomal escape occurs in a manner that is sufficient to induce GAL9-recruitment.

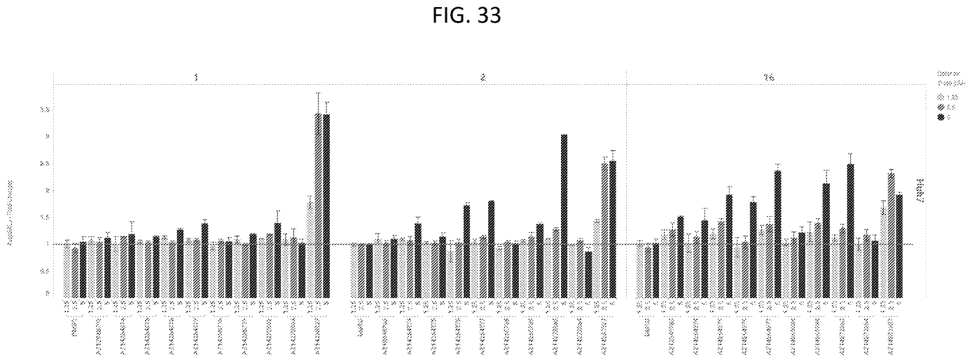

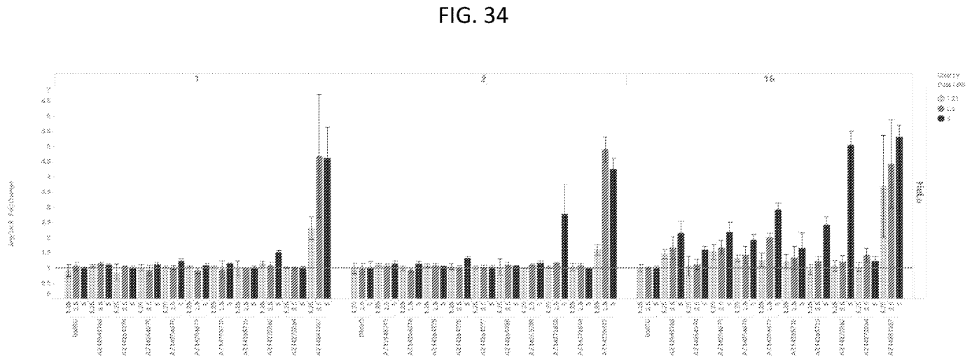

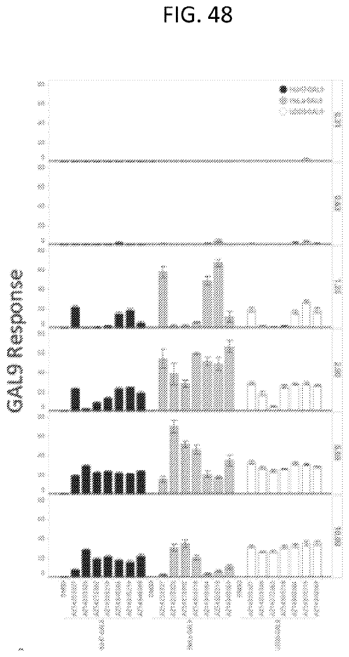

HeLa and Huh7 cells were further submitted to the mCherry-GAL9-recruitment analysis with treatment with select compounds and observed for endosomal rupture at various time points (1 hour, 2 hours, and 16 hours). GAL9 response results at different doses (1.25-5 μM) of the selected compounds are shown in FIG. 33 (Huh7 cells) and FIG. 34 (HeLa cells). As shown in both FIG. 33 and FIG. 34, AZ3327 (Compound 4) provides the most potent response indicative of endosomal remodeling from 1-hour post-dosing with AZ2862 (Compound 8) showing the second most potent of the exemplified compounds. FIG. 40 further provides additional results comparing time (0.25 to 4 hours), dose of select compounds (0.31 to 10 μM) and GAL9 response in Huh7 cells. As shown in FIG. 40, AZ3327 (Compound 4) is the most potent with GAL9+ responses from 1.25p M+ after 15 mins. AZ2862 also potently induces GAL9+ remodelling from 2.5 μM+. FIG. 41 and FIG. 48 provide additional data showing the interplay between dose (0.31 to 10 μM) and GAL9 response between different cell lines at 2 hours post-dosing. Potent compounds such as AZ3327 (Compound 4) show GAL9 responses across multiple cell lines of diverse background origins.

### Example 7. Nanoparticle Tracking Analysis

Many SSO transfection reagents work through complexation or association with the SSO. A nanoparticle tracking analysis (NTA) was performed to determine whether Compound 2 induced aggregation of oligonucleotides in cell media.

SSO was incubated in cell growth media with and without Compound 2 at 37° C. for 2 hours, mimicking the treatment plan shown in FIG. 10. Analysis was carried out with a Nanosight NS500 instrument running NTA 2.3 analytical software. A script that records 5×30 second videos in light-scatter mode was run with a camera level of 13. Analysis of the videos was then performed with screen gain set to 10 and a detection threshold at 7.

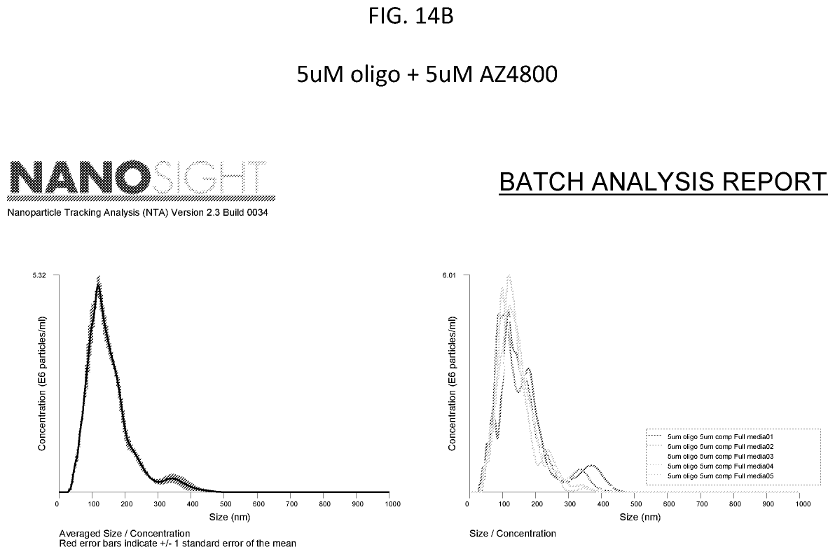

No significant differences in particle size were detected (FIG. 14A and FIG. 14B).

### Example 8. MALAT1 Activity Improvement with SMCs

### Cell Culture

N2A cells were cultured in Dulbecco's modified eagle medium (Gibco, 31,966) containing 10% fetal bovine serum (Gibco, 10,270) and were maintained at 37° C. in a humidified atmosphere containing 5% CO₂, 21% O₂.

### Cell Lysis, Reverse Transcription, and Real-Time Polymerase Chain Reaction

For knockdown analysis of the gapmers, 24-well plates 50,000 N2A cells in 500 μL total volume of media per well. Twenty-four hours later, the cells were treated with 2 μM MALAT1-targeting gapmer oligonucleotide having the sequence GM5CAttm5ctaatagm5cAGM5C, where m5c is 5-methylcytidine and capital letters are LNA nucleosides (SEQ ID NO:4) (Oligo Only), 2 μM MALA T1-targeting gapmer oligonucleotide and simultaneously 2 μM AZ3327 (2 μM AZ3327), or Lipofectamine-complexed MALAT1 Oligonucleotide at 200 nM (LF2000). All treatments were performed in biological triplicates. Twenty-four hours later, the gapmer-containing medium was removed from the wells, and the cells were washed once with Dulbecco's phosphate buffer saline (Gibco,14,040). RNA isolation was performed with the Promega Maxwell RSC RNA extraction instrument as per manufacturer's instructions. RNA concentration was measured via Nanodrop and cDNA was synthesized with High Capacity cDNA Reverse Transcription kit as per manufacturer's instructions (ThermoFisher Scientific, 4368814).

Real-time PCR reactions were run on QuantStudio 7 Flex Real-Time PCR System (Applied Biosystems) using MALAT1 TaqMan assays (mouse Mm01227912, Applied Biosystems) following the manufacturer's instructions. PCR reactions were run in technical triplicates using primer and probe sequences as follows: GAGGAATCAGATGAGGATATGGGA (forward sequence) (SEQ ID NO:5), AAGCAGGCTGACTTGGTTGC (reverse sequence) (SEQ ID NO:6), and TCGGTCTCTTCGACTAATCCCGCCAA (probe sequence) (SEQ ID NO:7). The absolute mRNA expression levels for MALAT1 were calculated by linear regression fitting the PCR product to a standard curve. The expression values were then normalized to the corresponding values from the negative control (untreated cells) and reported as a percentage.

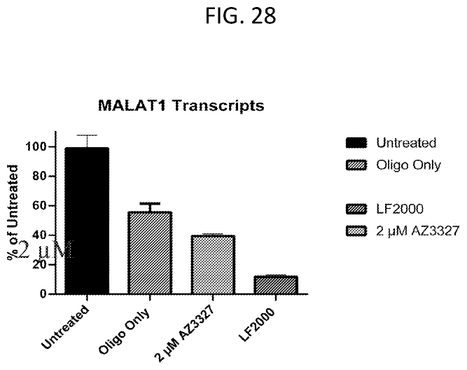

As shown in FIG. 28, addition of 2 μM AZ3327 (Compound 4) enhanced knockdown of MALAT1 mRNA expression as compared to oligo alone.

### Example 9. RNA Silencing Assays

Peptidylpropyl isomerase B (PPIB) mRNA was used in the assay below having a sequence:

PPIB siRNA:

Guide strand:
(SEQ ID NO: 8)
5′-UCACGAUGGAAUUUGCUGUU-3′
Passenger strand:
(SEQ ID NO: 9)
5′-CAGCAAAUUCCAUCGUGA-3′

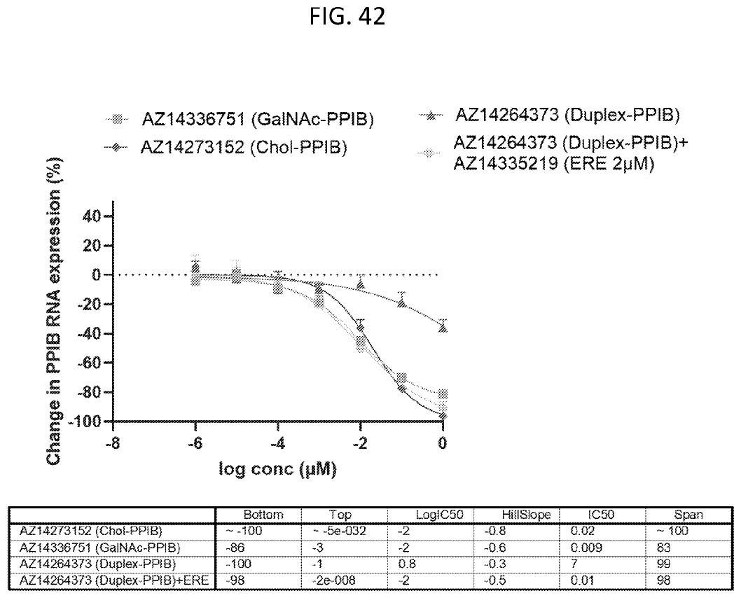

In vitro silencing of peptidylpropyl isomerase B (PPIB) mRNA in hepatocytes by lipid-conjugated siRNA or siRNA co-administered with 2 μM of an endosomal release enhancer (ERE) compound, Compound 32. Primary human hepatocytes (PHH) were 2D plated at 50,000 cells per well and cultured in serum free medium. At Day 1, the cells were treated with (i) unconjugated siRNA (“Duplex-PPIB”); (ii) siRNA conjugated with cholesterol (“Chol-PPIB”); (iii) siRNA conjugated GalNAc (“GalNAc-PPIB”); and (iv) unconjugated siRNA co-administered with 2 μM Compound 32 (“Duplex-PPIB+ERE”). siRNA concentrations are shown in Table 4. Conditions were performed in triplicate. Medium was changed on Day 2, and sample was collected for analysis on Day 4. Results are shown in FIG. 42.

TABLE 4
Tested siRNA Concentrations

| Concentration (μM) | Log Concentration (μM) |
|---|---|
| 1 | 0 |
| 0.1 | −1 |
| 0.01 | −2 |
| 0.001 | −3 |
| 0.0001 | −4 |
| 0.00001 | −5 |
| 0.000001 | −6 |

As shown in FIG. 42, co-administration of siRNA with an ERE provided similar change in expression of the target PPIB RNA as siRNA conjugated to cholesterol or GalNAc moiety. Co-administration of siRNA with ERE and siRNA conjugated with cholesterol or GalNAc decreased expression of the target PPIB RNA to a greater extent as compared to unconjugated siRNA alone.

### Example 10. Co-Delivery of Cre Recombinase with Compound 4 for In Vitro Editing

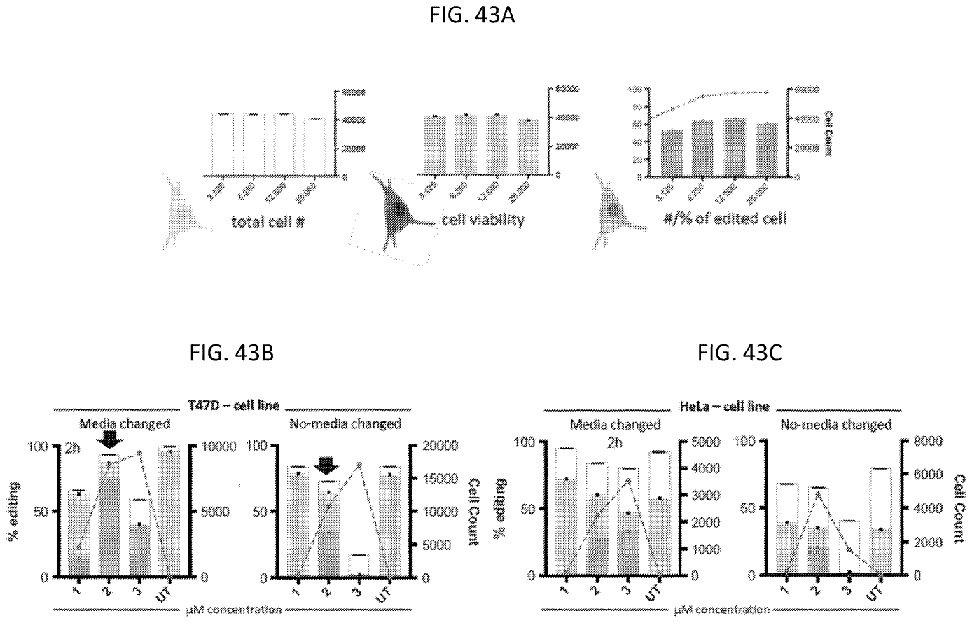

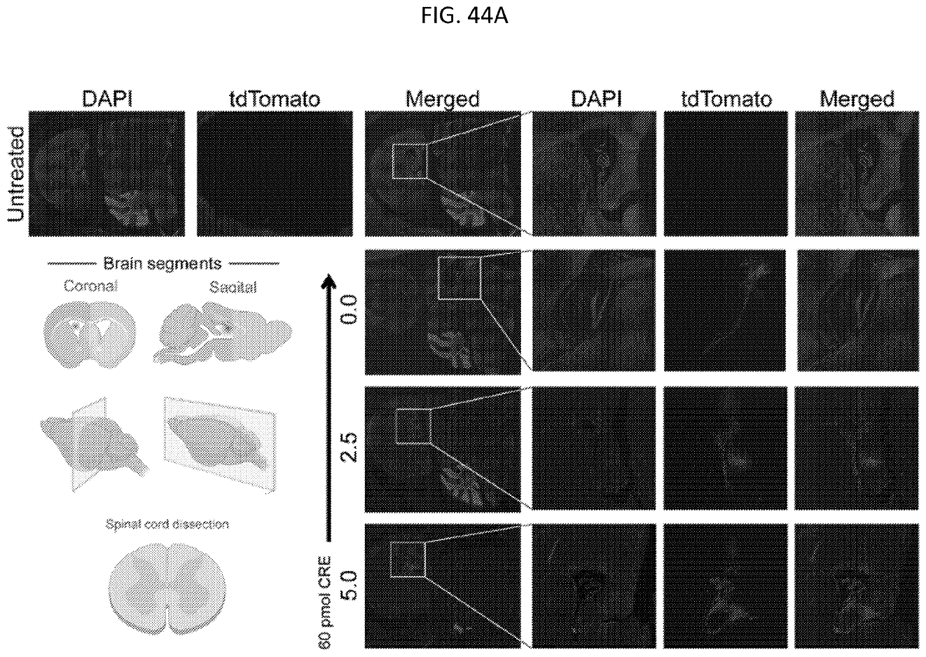

Cre recombinase was co-administered with Compound 4 to assess improvement in editing efficiency in an in vitro reporter system in which edited cells express GFP. FIG. 43A shows a legend for the data shown in FIGS. 43B and 43C. Total cell counts are shown as unfilled bars (FIG. 43A, left panel); viable cells are shown in light gray shaded bars (FIG. 43A, middle panel); edited cell counts are shown in green shaded bars, and edited cell percentage is shown as the dotted line (FIG. 43A, right panel).

FIG. 43B shows the results of T-47D cells co-administered with 50 nM Cre protein and 0, 1, 2, or 3 μM Compound 4, with either media changed after 2 hours (left panel) or unchanged (right panel). The media change increased cell viability, as shown by the difference between the left and right panels of FIGS. 43B and 43C. Further, as shown in FIG. 43B, co-administration of Compound 4 improved editing efficiency in T-47D cells in a dose-dependent manner. Greater than 80% editing was observed when Cre protein was co-administered with 2 μM or 3 μM Compound 4 in the media changed condition, and 3 μM Compound 4 in the media unchanged condition. Similar trends were observed when the experiment was repeated in HeLa cells, as shown in FIG. 43C.

### Example 11. Co-Delivery of Cre Recombinase with Compound 4 for In Vivo and Ex Vivo Editing

Cre recombinase (MGSSHHHHHHSSGLVPRGSHGGGSAAAMGTRLPKKKRKVSNLLTVHQNLPALPVD ATSDEVRKNLMDMFRDRQAFSEHTWKMLLSVCRSWAAWCKLNNRKWFPAEPEDV RDYLLYLQARGLAVKTIQQHLGQLNMLHRRSGLPRPSDSNAVSLVMRRIRKENVDA GERAKQALAFERTDFDQVRSLMENSDRCQDIRNLAFLGIAYNTLLRIAEIARIRVKDI SRTDGGRMLIHIGRTKTLVSTAGVEKALSLGVTKLVERWISVSGVADDPNNYLFCRV RKNGVAAPSATSQLSTRALEGIFEATHRLIYGAKDDSGQRYLAWSGHSARVGAARD MARAGVSIPEIMQAGGWTNVNIVMNYIRNLDSETGAMVRLLEDGD (SEQ ID NO: 10)) was co-administered with Compound 4 to assess improvement in editing efficiency in an in vivo reporter system, in which edited mice express tdTomato. Floxed mice were administered via intracerebroventricular injection (ICV) with 60 pmol Cre recombinase and varying concentrations (2.5, 5.0, 7.5, 10.0, 12.5, and 15 μM) of Compound 4 as calculated in 40 μL CSF volume. Brain segments were analyzed by immunofluorescent staining 7 days post-treatment.

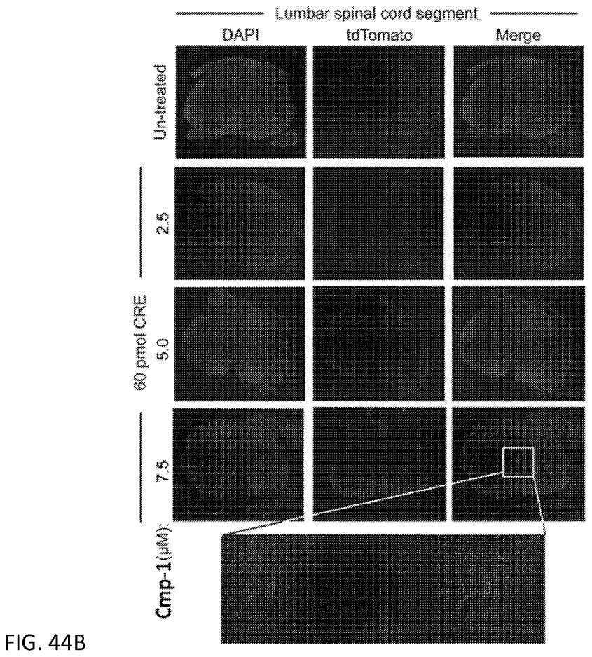

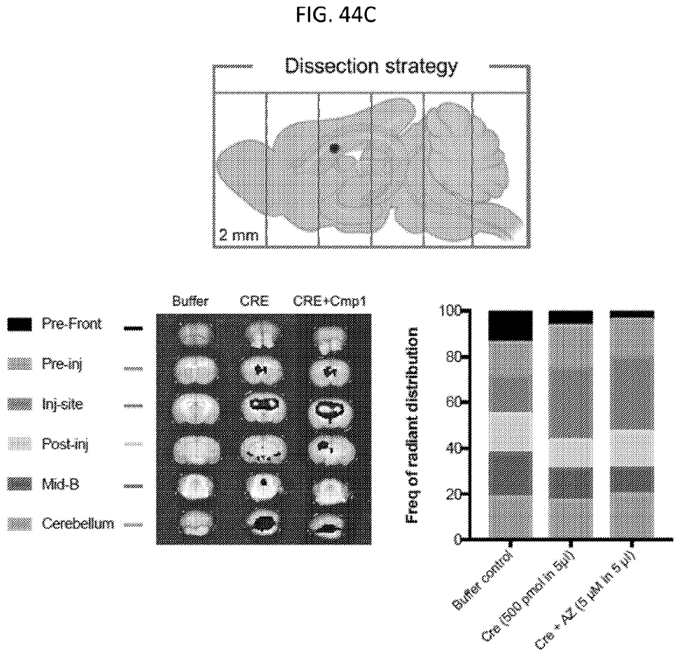

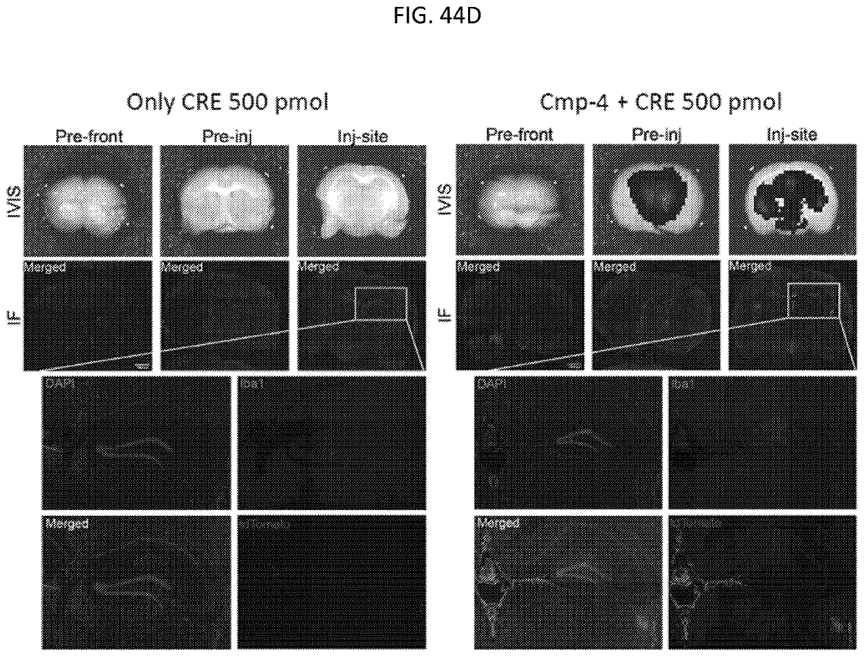

FIG. 44A shows coronal and sagittal brain segments, while FIG. 44B shows lumbar spinal cord segment. Both FIGS. 44A and 44B show increased tdTomato staining at higher concentrations of Compound 4, demonstrating that in vivo editing in the brain is enhanced by Compound 4 in a dose-dependent manner. Further, only mice treated with Compound 4 presented obvious staining in the lumbar sections of the spinal cord.

FIG. 44C shows the dissection strategy and results, indicating that editing was observed primarily at the injection site. FIG. 44D further confirms the editing via IVIS® in vivo imaging and immunofluorescence staining.

The combination of Cre recombinase with Compound 4 was also tested in ex vivo editing of anti-CD3/28 stimulated T cells. Cells were obtained from mice then subjected to anti-CD3/28 stimulation, then treated with 2.5 μM of Compound 4 with varying concentrations of Cre recombinase (50, 100, 200, 500 and 1000 nM), either on a U-plate with no mixing (T1) or mixed in a tube shaker during treatment (T2).

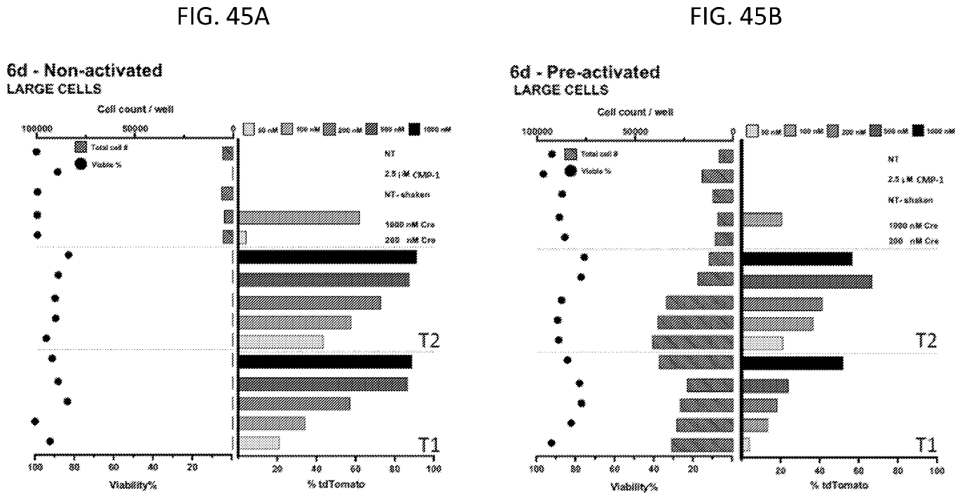

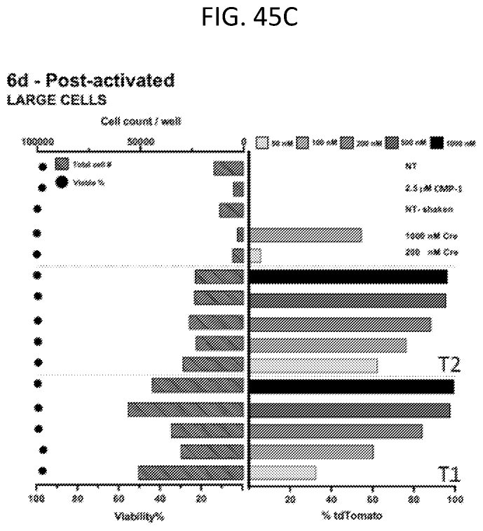

Percent viability and editing efficiency of non-activated, pre-activated, and post-activated cells are shown in FIGS. 45A-C, respectively. FIG. 45C shows greater than 90% editing efficiency in activated T cells without affecting proliferative capacity. FIG. 45A shows that non-activated T cells were also highly edited, however, the proliferation rate was compromised due to the nature of the experiment.

### Example 12. Co-Delivery of Base Editor with Compound 4

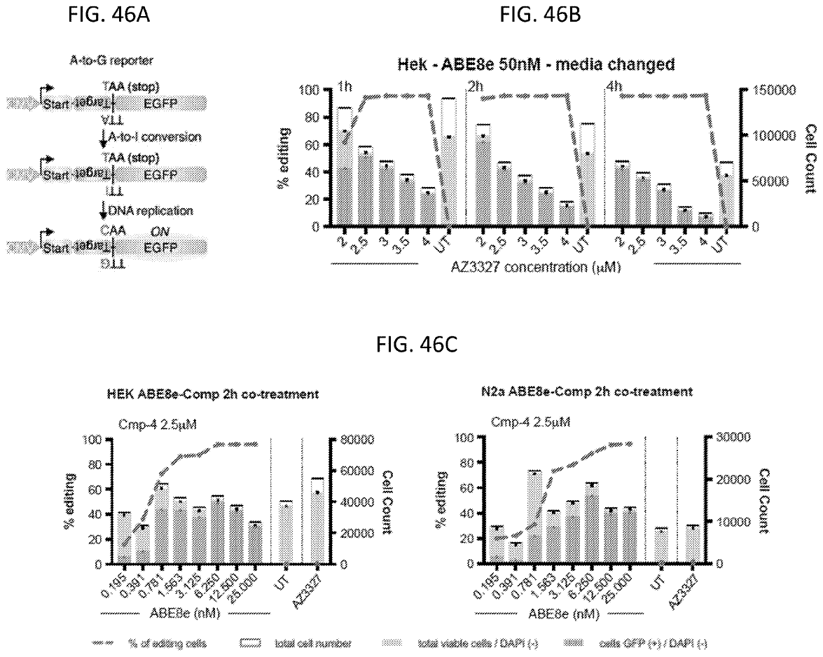

The ABE8e base editor comprising SpCas9 D10A nickase and a modified TadA adenosine deaminase (Richter et al., Nature Biotechnol. 38:883-891 (2020)) was co-administered with Compound 4 to assess improvement in editing efficiency in an in vitro reporter system in which edited cells express GFP. FIG. 46A shows a schematic for the editing-based reporter system, which converts a TAA stop codon into a CAA codon, thereby enabling transcription of the GFP gene. The bar shading and lines showing total cell counts, viable cells, and edited cells are the same as in FIG. 43A.

HEK cells were co-administered with a fixed concentration (50 nM) of ABE8e and varying concentrations of AZ3327 (Compound 4). The results in FIG. 46B show greater than 90% editing efficiency at Compound 4 concentrations of above 2.5 μM. The left, middle, and right panels of FIG. 46B shows media changed at 1 hour, 2 hours, and 4 hours post co-administration, respectively.

HEK and N2a cells were co-administered with a fixed concentration (2.5 μM) of Compound 4 and varying concentrations of ABE8e. The results in FIG. 46C (left panel: HEK; right panel: N2a) show ABE8e-dose-dependent editing efficiency, where ABE8e concentrations lower than 5 nM showed reduced efficiency.

1. A compound comprising a structure represented by Formula I: 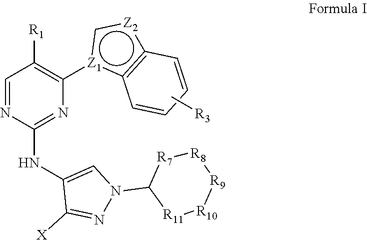
wherein
one of Z₁ and Z₂ is N, and the other is C;
R₁ is hydro, halo, C₁-C₄ alkyl, —OR₄, —C(═O)NR₅R₅, —CO₂R₆, or cyano;
X is hydro, C₁-C₄ alkyl, or —OR₂;
R₂ is hydro or C₁-C₄ alkyl;
R₃ is hydro, halo, C₁-C₄ alkyl, —(CH₂)_{y}OH, —OR₄, —C(═O)NR₅R₅, —CO₂R₆, or cyano;
R₄ is C₁-C₄ alkyl;
each R₅ is independently hydro or C₁-C₄ alkyl,
R₆ is hydro or C₁-C₄ alkyl,
R₇, R₈, R₉, R₁₀, R₁₁ are each independently CHR₁₂, CR₁₂R₁₇ or NR₁₃;
R₁₂ is hydro, C₁-C₄ alkyl, —OR₁₄, or CO₂R₁₅;
R₁₃ is hydro, C₁-C₄ alkyl, —(CH₂)_{y}OH, —OR₁₄, —CO₂R₁₅, or —C(═O)R₁₆;
R₁₄ is hydro or C₁-C₄ alkyl;
R₁₅ is hydro or C₁-C₄ alkyl;
R₁₆ is hydro or C₁-C₄ alkyl;
R₁₇ is hydro or C₁-C₄ alkyl;
y is 0, 1, 2, or 3; and
wherein one or more of the alkyl are optionally substituted with one or more halo; or a pharmaceutically acceptable salt thereof.

2. The compound of claim 1, wherein the compound comprises a structure represented by Formula Ia:

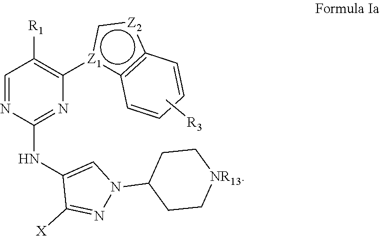

3. The compound of claim 1, wherein the compound comprises a structure represented by Formula Ib or Formula Ic:

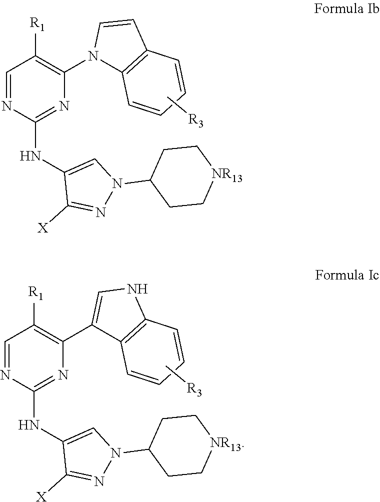

4. The compound of any one of claims 1 to 3, wherein R₁ is cyano, halo, or C₁-C₄ alkyl optionally substituted with one or more chloro or fluoro.

5. The compound of any one of claims 1 to 3, wherein R₁ is selected from: cyano, methyl, ethyl, trifluoromethyl, difluoromethyl, fluoromethyl, trichloromethyl, dichloromethyl, chloromethyl, fluoro, chloro, and bromo.

6. The compound of any one of claims 1 to 3, wherein R₁ is cyano, bromo, chloro, fluoro, or trifluoromethyl.

7. The compound of any one of claims 1 to 3, wherein R₁ is fluoro.

8. The compound of any one of claims 1 to 7, wherein X is —OR₂, and R₂ is methyl, ethyl, or isopropyl.

9. The compound of any one of claims 1 to 7, wherein X is hydro.

10. The compound of any one of claims 1 to 9, wherein R₃ is hydro; cyano; C₁-C₄ alkyl optionally substituted with one or more chloro, fluoro, or hydroxy; or —OR₄, wherein R₄ is C₁-C₄ alkyl.

11. The compound of claim 1, wherein R₇, R₈, R₁₀, and R₁₁ are each independently CHR₁₂, and R₉ is NR₁₃.

12. The compound of claim 11, wherein each R₁₂ is hydro.

13. The compound of any one of claims 2 to 12, wherein R₁₃ is hydro, C₁-C₄ alkyl, —C(═O)R₁₆, or —(CH₂)_{y}OH, wherein y is 1, 2 or 3.

14. The compound of claim 13, wherein R₁₃ is —(CH₂)₃OH.

15. The compound of claim 13, wherein R₁₃ is methyl.

16. The compound of claim 2 or 3, wherein R₁ is halo, X is —OR₂, R₂ is ethyl or methyl, R₃ is cyano, and R₁₃ is hydro, C₁-C₄ alkyl, —C(═O)CH₃, or —(CH₂)₃OH.

17. The compound of claim 2 or 3, wherein R₁ is haloalkyl, X is —OR₂, R₂ is ethyl or methyl, R₃ is cyano, and R₁₃ is hydro or methyl.

18. The compound of claim 2 or 3, wherein R₁ is halo, X is —OR₂, R₂ is ethyl or methyl, R₃ is methyl or methoxy, and R₁₃ is hydro.

19. The compound of claim 2 or 3, wherein R₁ is halo, X is —OR₂, R₂ is C₁-C₄ alkyl, R₃ is hydro, and R₁₃ is hydro.

20. The compound of claim 2 or 3, wherein R₁ is haloalkyl, X is —OR₂, R₂ is ethyl or methyl, R₃ is hydro, and R₁₃ is hydro or methyl.

21. The compound of claim 2 or 3, wherein R₁ is halo, X is hydro, R₃ is cyano, and R₁₃ is hydro.

22. The compound of claim 2 or 3, wherein R₁ is cyano, X is —OR₂, R₂ is ethyl or methyl, R₃ is haloalkyl, and R₁₃ is hydro.

23. The compound of claim 2 or 3, wherein R₁ is ethyl or methyl, X is —OR₂, R₂ is ethyl or methyl, R₃ is hydro, and R₁₃ is hydro.

24. The compound of claim 2 or 3, wherein R₁ is halo, X is —OR₂, R₂ is ethyl or methyl, R₃ is (CH₂)_{y}OH, and R₁₃ is hydro, wherein y is 0, 1, 2, or 3.

25. The compound of claim 2 or 3, wherein R₁ is fluoro, X is —OR₂, R₂ is methyl, R₃ is cyano, and R₁₃ is hydro.

26. The compound of claim 2 or 3, wherein R₁ is fluoro, X is —OR₂, R₂ is ethyl, R₃ is cyano, and R₁₃ is methyl.

27. The compound of claim 1, wherein Z₁ is N, R₁ is fluoro, X is —OR₂, R₂ is methyl, R₃ is cyano, R₁₃ is hydro, each of R₇, R₇, R₈, and R₁₁ are CH₂, and R₈ is CR₁₂R₁₇, wherein each of R₁₂ and R₁₇ is methyl.

28. The compound of any one of claims 1 to 3, wherein the compound is represented by any one of the following Compounds 2-8 or 32: 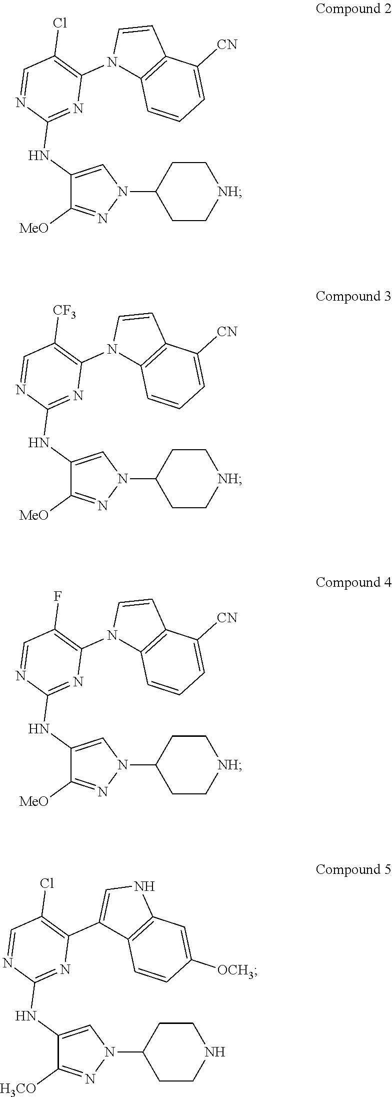 [SMILES: COc1nn(C2CCNCC2)cc1Nc1ncc(Cl)c(-n2ccc3c(C#N)cccc32)n1 ; COc1nn(C2CCNCC2)cc1Nc1ncc(C(F)(F)F)c(-n2ccc3c(C#N)cccc32)n1 ; COc1nn(C2CCNCC2)cc1Nc1ncc(F)c(-n2ccc3c(C#N)cccc32)n1 ; COc1ccc2c(-c3nc(Nc4cn(C5CCNCC5)nc4OC)ncc3Cl)c[nH]c2c1] 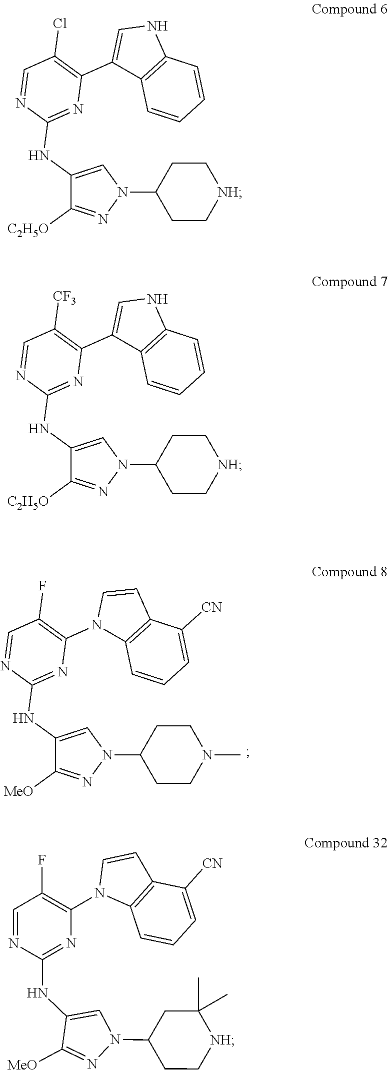
or a pharmaceutically acceptable salt thereof.

29. The compound of any one of claims 1 to 3, wherein the compound is Compound 4: 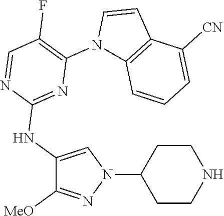 [SMILES: COc1nn(C2CCNCC2)cc1Nc1ncc(F)c(-n2ccc3c(C#N)cccc32)n1]
or a pharmaceutically acceptable salt thereof.

30. The compound of any one of claims 1 to 3, wherein the compound is Compound 8: 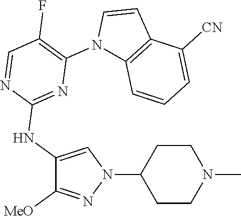 [SMILES: COc1nn(C2CCN(C)CC2)cc1Nc1ncc(F)c(-n2ccc3c(C#N)cccc32)n1]
or a pharmaceutically acceptable salt thereof.

31. The compound of claim 1, wherein the compound is Compound 32: 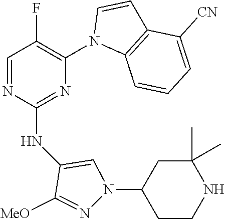 [SMILES: COc1nn(C2CCNC(C)(C)C2)cc1Nc1ncc(F)c(-n2ccc3c(C#N)cccc32)n1]
or a pharmaceutically acceptable salt thereof.

32. A pharmaceutical composition comprising:
an oligonucleotide and/or a polypeptide;
a compound according to any of claims 1 to 31; and
a pharmaceutically acceptable diluent or carrier.

33. The pharmaceutical composition of claim 32, wherein the oligonucleotide is single-stranded.

34. The pharmaceutical composition of claim 32, wherein the oligonucleotide is double stranded.

35. The pharmaceutical composition of any one of claims 32 to 34, wherein the oligonucleotide comprises DNA.

36. The pharmaceutical composition of any one of claims 32 to 34, wherein the oligonucleotide comprises RNA.

37. The pharmaceutical composition of any one of claims 32 to 36, wherein the oligonucleotide comprises from about 8 to about 30 nucleotides.

38. The pharmaceutical composition of any of claims 32 to 37, wherein the oligonucleotide is an antisense oligonucleotide (ASO), a splice switching oligonucleotide (SSO), interfering RNA (RNAi), small interfering RNA (siRNA), micro RNA (miRNA), an antagomir, a decoy oligonucleotide, or a combination thereof.

39. The pharmaceutical composition of any of claims 32 to 38, wherein the oligonucleotide comprises one or more modified nucleotides.

40. The pharmaceutical composition of claim 39, wherein the one or more modified nucleotides comprise phosphodiester (PO); phosphorothioate (PS); 2′O-methyl (2′OMe); 2′O-methoxyethyl (MOE); peptide nucleic acid (PNA); phosphoroamidate morpholino (PMO); locked nucleic acid (LNA); 2′-deoxy-2′-fluoro (2′-F); any other 2′ modified oligonucleotide; or a combination thereof.

41. A method of introducing an oligonucleotide and/or a polypeptide into a nucleus and/or cytosol of a cell, the method comprising:
(a) contacting the cell with the oligonucleotide and/or the polypeptide; and
(b) contacting the cell with a compound according to any one of claims 1 to 31, wherein the compound facilitates entry of the oligonucleotide and/or the polypeptide into a nucleus and/or cytosol of the cell.

42. The method of claim 41, wherein the oligonucleotide and/or the polypeptide is internalized by the cell through endocytosis and encapsulated within an endosome, and the compound facilitates release of the oligonucleotide and/or the polypeptide from the endosome.

43. The method of claim 41, wherein the oligonucleotide and/or the polypeptide is internalized by the cell through transient pore formation, and the compound facilitates transient pore formation in the plasma membrane.

44. The method of any of claims 41 to 43, wherein contacting the cell with the oligonucleotide and/or the polypeptide of (a) is performed in a composition, wherein the composition of (a) comprises about 0.025 μM to about 20 μM of the oligonucleotide and/or the polypeptide.

45. The method of claim 44, wherein the composition of (a) comprises about 0.1 μM to about 10 μM, about 0.1 μM to about 5 μM, or about 0.1 μM to about 1 μM of the oligonucleotide and/or the polypeptide.

46. The method of any of claims 41 to 45, wherein the cell is contacted with a second composition comprising about 1 μM to about 20 μM of the compound.

47. The method of claim 46, wherein contacting the cell with the compound of (b) is performed in a composition, wherein the composition of (b) comprises about 1 μM to about 10 μM of the compound.

48. The method of claim 47, wherein the composition of (b) comprises at least about 1 μM, about 1.5 μM, about 2 μM, about 2.5 μM, about 3 μM, about 3.5 μM, about 4 μM, about 4.5 μM, or about 5 μM and up to about 6 μM, about 6.5 μM, about 7 μM, about 7.5 μM, about 8 μM, about 8.5 μM, about 9 μM, about 9.5 μM, or about 10 μM of the compound.

49. The method of any of claims 41 to 48, wherein the cell is contacted with the oligonucleotide and/or the polypeptide of (a) and the compound of (b) at approximately the same time.

50. The method of any of claims 41 to 48, wherein the cell is contacted with the oligonucleotide and/or the polypeptide of (a) before being contacted with the compound of (b).

51. The method of claim 50, wherein the cell is contacted with the oligonucleotide and/or the polypeptide of (a) up to about 48 hours before being contacted with the compound of (b).

52. The method of claim 50, wherein the cell is contacted with the oligonucleotide and/or the polypeptide of (a) about 12 hours to about 48 hours before being contacted with the compound of (b).

53. The method of any of claims 41 to 48, wherein the contacting of (a) and the contacting of (b) are performed in the same composition.

54. The method of claim 53, wherein the composition comprises about 0.025 μM to about 10 μM of the oligonucleotide and/or the polypeptide.

55. The method of claim 54, wherein the composition comprises about 0.1 μM to about 5 μM, or about 0.1 μM to about 1 μM of the oligonucleotide and/or the polypeptide.

56. The method of any of claims 53 to 55, wherein the composition comprises about 1 μM to about 20 μM of the compound.

57. The method of claim 56, wherein the composition comprises about 1 μM to about 10 μM of the compound.

58. The method of claim 57, wherein the composition comprises at least about 1 μM, about 1.5 μM, about 2 μM, about 2.5 μM, about 3 μM, about 3.5 μM, about 4 μM, about 4.5 μM, or about 5 μM and up to about 6 μM, about 6.5 μM, about 7 μM, about 7.5 μM, about 8 μM, about 8.5 μM, about 9 μM, about 9.5 μM, or about 10 μM of the compound.

59. The method of any of claims 41 to 58, wherein the oligonucleotide hybridizes to a target nucleic acid in the cell.

60. The method of claim 59, wherein the target nucleic acid is in the nucleus of the cell.

61. The method of claim 59, wherein the target nucleic acid is in the cytosol of the cell.

62. The method of any of claims 41 to 61, wherein contacting the cell with the compound of (b) results in endosomal membrane permeabilization as determined by mCherry-GAL9 recruitment assay.

63. The method of any of claims 41 to 62, wherein the oligonucleotide and/or the polypeptide alters activity of a gene expressed by the cell.

64. The method of claim 63, wherein the oligonucleotide and/or the polypeptide increases activity of a gene expressed by the cell.

65. The method of claim 64, wherein the activity of the gene expressed by the cell is increased at least about 10× when the cell is contacted with the oligonucleotide and/or the polypeptide and the compound, as compared to a cell that is contacted with the oligonucleotide and/or the polypeptide and not the compound.

66. The method of claim 65, wherein the activity of the gene expressed by the cell is increased at least about 100× when the cell is contacted with the oligonucleotide and/or the polypeptide and the compound, as compared to a cell that is contacted with the oligonucleotide and/or the polypeptide and not the compound.

67. The method of claim 63, wherein the oligonucleotide and/or the polypeptide decreases activity of a gene expressed by the cell.

68. The method of claim 67, wherein the activity of the gene expressed by the cell is decreased at least about 10× when the cell is contacted with the oligonucleotide and/or the polypeptide and the compound, as compared to a cell that is contacted with the oligonucleotide and/or the polypeptide and not the compound.

69. The method of claim 68, wherein the activity of the gene expressed by the cell is decreased at least about 100× when the cell is contacted with the oligonucleotide and/or the polypeptide and the compound, as compared to a cell that is contacted with the oligonucleotide and/or the polypeptide and not the compound.

70. The method of any of claims 41 to 69, comprising in vitro delivery of the oligonucleotide and/or the polypeptide to the cell.

71. The method of any of claims 41 to 69, comprising in vivo delivery of the oligonucleotide and/or the polypeptide to the cell.

72. The method of any of claims 41 to 69, wherein the cell is a cultured cell.

73. The method of any of claims 41 to 69, wherein the cell is an isolated cell obtained from a subject in need of treatment.

74. The method of any of claims 41 to 69, wherein the cell is part of a tissue or organ.

75. The method of any of claims 41 to 69, wherein the cell is a mammalian cell.

76. A method of altering expression of a target nucleic acid in a cell, the method comprising:
contacting the cell with an oligonucleotide that is capable of hybridizing to the target nucleic acid, wherein the oligonucleotide is internalized by the cell through endocytosis and encapsulated within an endosome; and
contacting the cell with a compound according to any of claims 1 to 31, wherein the compound facilitates release of the oligonucleotide from the endosome, wherein the hybridization of the oligonucleotide to the target nucleic acid alters expression of the target nucleic acid.

77. The method of claim 76, wherein hybridization of the oligonucleotide to the target nucleic acid increases expression of the target nucleic acid.

78. The method of claim 76, wherein hybridization of the oligonucleotide to the target nucleic acid decreases expression of the target nucleic acid.

79. A method of releasing an oligonucleotide and/or a polypeptide from an endosome, the method comprising:
contacting the cell with the oligonucleotide and/or the polypeptide, wherein the oligonucleotide and/or the polypeptide is internalized by the cell through endocytosis and encapsulated within the endosome; and
contacting the cell with a compound according to any of claims 1 to 31, wherein the compound facilitates release of the oligonucleotide and/or the polypeptide from the endosome.

80. A method for the treatment and/or prevention of a disorder in a subject comprising:
administering to the subject (i) a therapeutically effective amount of an oligonucleotide and/or a polypeptide and (ii) an effective amount of a compound of any of claims 1 to 31.

81. The method of claim 80, wherein the compound is administered to the subject concurrently with the oligonucleotide and/or the polypeptide.

82. The method of claim 80, wherein the compound is administered to the subject after administration of the oligonucleotide and/or the polypeptide.

83. The method of claim 82, wherein the compound is administered to the subject up to about 48 hours after administration of the oligonucleotide and/or the polypeptide.

84. The method of claim 82, wherein the compound is administered to the subject about 12 hours to about 48 hours after administration of the oligonucleotide and/or the polypeptide.

85. The method of any of claims 80 to 79, wherein administration comprises parenteral administration.

86. The method of claim 85, wherein administration comprises intravenous or subcutaneous administration.

87. The method of any of claims 80 to 86, wherein the subject is a mammal.

88. The method of any of claims 80 to 86, wherein the subject is a human.

89. A method for the treatment and/or prevention of a disorder in a subject comprising:
isolating a cell from the subject;
contacting the isolated cell with a therapeutically effective amount of (i) an oligonucleotide and/or a polypeptide and (ii) an effective amount of a compound of any of claims 1 to 31 to produce an engineered cell; and transplanting the engineered cell in the subject.

90. The method of claim 89, wherein the isolated cell is contacted with a composition comprising about 0.025 μM to about 20 μM of the oligonucleotide and/or the polypeptide.

91. The method of claim 90, wherein the composition comprises about 0.1 μM to about 10 μM, about 0.1 μM to about 5 μM, or about 0.1 μM to about 1 μM of the oligonucleotide and/or the polypeptide.

92. The method of any of claims 89 to 91, wherein the isolated cell is contacted with a composition comprising about 1 μM to about 20 μM of the compound.

93. The method of claim 92, wherein the composition comprises about 1 μM to about 10 μM of the compound.

94. The method of claim 93, wherein the composition comprises at least about 1 μM, about 1.5 μM, about 2 μM, about 2.5 μM, about 3 μM, about 3.5 μM, about 4 μM, about 4.5 μM, or about 5 μM and up to about 6 μM, about 6.5 μM, about 7 μM, about 7.5 μM, about 8 μM, about 8.5 μM, about 9 μM, about 9.5 μM, or about 10 μM of the compound.

95. The method of any of claims 89 to 92, wherein the isolated cell is contacted with (i) the oligonucleotide and/or the polypeptide and (ii) the compound at approximately the same time.

96. The method of claim 95, comprising contacting the isolated cell with a composition comprising (i) the oligonucleotide and/or the polypeptide and (ii) the compound.

97. The method of any of claims 89 to 92, wherein the isolated cell is contacted with the oligonucleotide and/or the polypeptide before the isolated cell is contacted with the compound.

98. The method of claim 97, wherein the isolated cell is contacted with the oligonucleotide and/or the polypeptide up to about 48 hours before the isolated cell is contacted with the compound.

99. The method of claim 98, wherein the isolated cell is contacted with the oligonucleotide and/or the polypeptide about 12 hours to about 48 hours the isolated cell is contacted with the compound.

100. The method of any of claims 89 to 99, wherein the subject is a mammal.

101. The method of any of claims 89 to 100, wherein the subject is a human.

102. Use of (i) a compound of any of claims 1 to 31 and (ii) an oligonucleotide and/or the polypeptide in the manufacture of a medicament for gene therapy.

103. A kit comprising (i) a compound of any of claims 1 to 31 and (ii) an oligonucleotide and/or the polypeptide.


SEQUENCE LISTING (10 sequences)

SEQ ID NO: 1 - RNA, 18 nt, synthetic construct
  misc_feature 1..18: note = Synthetic oligonucleotide
  source 1..18: mol_type = other RNA
cctcttacctcagttaca

SEQ ID NO: 2 - DNA, 24 bp, synthetic construct
  misc_feature 1..24: note = Primer
  source 1..24: mol_type = other DNA
ttgatatgtggatttcgagtcgtc

SEQ ID NO: 3 - DNA, 23 bp, synthetic construct
  misc_feature 1..23: note = Primer
  source 1..23: mol_type = other DNA
tgtcaatcagagtgcttttggcg

SEQ ID NO: 4 - DNA, 16 bp, synthetic construct
  misc_feature 1..16: note = Synthetic oligonucleotide
  modified_base 1..3: mod_base = OTHER; note = locked nucleic acid
  modified_base 2: mod_base = m5c
  modified_base 6: mod_base = m5c
  modified_base 13: mod_base = m5c
  modified_base 14..16: mod_base = OTHER; note = locked nucleic acid
  modified_base 16: mod_base = m5c
  source 1..16: mol_type = other DNA
gcattctaatagcagc

SEQ ID NO: 5 - DNA, 24 bp, synthetic construct
  misc_feature 1..24: note = Primer
  source 1..24: mol_type = other DNA
gaggaatcagatgaggatatggga

SEQ ID NO: 6 - DNA, 20 bp, synthetic construct
  misc_feature 1..20: note = Primer
  source 1..20: mol_type = other DNA
aagcaggctgacttggttgc

SEQ ID NO: 7 - DNA, 26 bp, synthetic construct
  misc_feature 1..26: note = Probe
  source 1..26: mol_type = other DNA
tcggtctcttcgactaatcccgccaa

SEQ ID NO: 8 - RNA, 20 nt, synthetic construct
  source 1..20: mol_type = other RNA
tcacgatggaatttgctgtt

SEQ ID NO: 9 - RNA, 18 nt, synthetic construct
  source 1..18: mol_type = other RNA
cagcaaattccatcgtga

SEQ ID NO: 10 - protein, 381 aa, synthetic construct
MGSSHHHHHHSSGLVPRGSHGGGSAAAMGTRLPKKKRKVSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD